(19) United States
(12) Patent Application Publication
(10) Pub. No.: US 2002/0009730 A1
(43) Pub. Date: Jan. 24, 2002

(54) Human stress array

(76) Inventors: Alex Chenchik, Palo Alto, CA (US); Matvey E. Lukashev, Newton, MA (US)

Correspondence Address: Bret E. Field, BOZICEVIC, FIELD & FRANCIS LLP, 200 Middlefield Road, Suite 200, Menlo Park, CA, 94025, US

(21) Appl. No.: 09/782,909

(22) Filed: Feb. 13, 2001

Related U.S. Application Data
(63) Continuation-in-part of application No. 09/441,920, filed on Nov. 17, 1999.

(51) Int. Cl. C12Q 1/68; C07H 21/04

(52) U.S. Cl. 435/6; 536/24.3

(57) ABSTRACT

Human stress arrays and methods for their use are provided. The subject arrays include a plurality of polynucleotide spots, each of which is made up of a polynucleotide probe composition of unique polynucleotides corresponding to a human stress gene. The subject arrays find use in hybridization assays, particularly in assays for the identification of differential gene expression of human stress genes.

### CROSS-REFERENCE TO RELATED APPLICATIONS

This application is a continuation-in-part of application Ser. No. 09/441,920 filed Nov. 17, 1999; application Ser. No. 09/440,305 filed Nov. 17, 1999; and application Ser. No. 09/222,256 filed Dec. 28, 1998; which applications are all continuation-in-part applications of application Ser. No. 09/053,375 filed on Mar. 31, 1998, the disclosure of which is herein incorporated by reference.

### INTRODUCTION

1. Technical Field

The field of this invention is biopolymeric arrays.

2. Background of the Invention

“Biochips” or arrays of binding agents, such as oligonucleotides and peptides, have become an increasingly important tool in the biotechnology industry and related fields. These binding agent arrays, in which a plurality of binding agents are deposited onto a solid support surface in the form of an array or pattern, find use in a variety of applications, including drug screening, nucleic acid sequencing, mutation analysis, and the like. One important use of biochips is in the analysis of differential gene expression, where the expression of genes in different cells, normally a cell of interest and a control, is compared and any discrepancies in expression are identified. In such assays, the presence of discrepancies indicates a difference in the classes of genes expressed in the cells being compared.

In methods of differential gene expression, arrays find use by serving as a substrate to which is bound nucleic acid “probe” fragments. One then obtains “targets” from analogous cells, tissues or organs of a healthy and diseased organism. The targets are then hybridized to the immobilized set of nucleic acid “probe” fragments. Differences between the resultant hybridization patterns are then detected and related to differences in gene expression in the two sources.

A variety of different array technologies have been developed in order to meet the growing need of the biotechnology industry, as evidenced by the extensive number of patents and references listed in the relevant literature section below.

Despite the wide variety of array technologies currently in preparation or available on the market, there is a continued need to identify new array devices to meet the needs of specific research applications.

Relevant Literature

Patents and patent applications describing arrays of biopolymeric compounds and methods for their fabrication include: 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,895; 5,624,711; 5,639,603; 5,658,734; 5,700,637; 5,744,305; 5,770,456; WO 93/17126; WO 95/11995; WO 95/35505; EP 742 287; and EP 799 897.

Patents and patent application describing methods of using arrays in various applications include: 5,143,854; 5,288,644; 5,324,633; 5,432,049; 5,470,710; 5,492,806; 5,503,980; 5,510,270; 5,525,464; 5,547,839; 5,580,732; 5,661,028; WO 95/21265; WO 96/31622; WO 97/10365; WO 97/27317; EP 373 203; and EP 785 280.

Other references of interest include: Atlas Human cDNA Expression Array I (Apr. 1997) CLONTECHniques XII: 4-7; Lockhart et al., Nature Biotechnology (1996) 14: 1675-1680; Shena et al., Science (1995) 270: 467-470; Schena et al., Proc. Nat'l Acad. Sci. USA (1996)93:10614-10619; Shalon et al., Genome Res. (1996) 6: 639-645; Milosavljevic et al., Genome Res. (1996) 6:132-141; Nguyen et al., Genomics (1995)29: 207-216; Piétu et al., Genome Res. (1996) 6: 492-503; Zhao et al., Gene (1995) 166:207-213; Chalifour et al., Anal. Biochem. (1994) 216:299-304; Heller et al., Proc. Nat=l Acad. Sci. USA (1997) 94: 2150-2155; and Schena, M., BioAssays (1996) 18: 427-431.

### SUMMARY OF THE INVENTION

Human stress arrays and kits including the same, as well as methods for their preparation and use in hybridization assays, are provided. The subject arrays have a plurality of probe polynucleotide spots each made up of a unique polynucleotide(s) that corresponds to a human gene associated with a stress response. The subject arrays find use in the expression analysis of human stress genes.

### DEFINITIONS

The term “nucleic acid” as used herein means a polymer composed of nucleotides, e.g. deoxyribonucleotides or ribonucleotides.

The terms “ribonucleic acid” and “RNA” as used herein means a polymer composed of ribonucleotides.

The terms “deoxyribonucleic acid” and “DNA” as used herein means a polymer composed of deoxyribonucleotides.

The term “oligonucleotide” as used herein denotes single stranded nucleotide multimers of from about 10 to 100 nucleotides in length.

The term “polynucleotide” as used herein refers to single or double stranded polymer composed of nucleotide monomers of greater than about 120 nucleotides in length up to about 1000 nucleotides in length.

“Human stress genes” and “human stress related genes” are those genes that have been identified by those of skill in the art as genes whose products modulate cellular responses to various stressors, including heat shock, irradiation, infection, and treatment with cytotoxic or genotoxic compounds. Preferably, the human stress genes are genes that are well characterized, i.e. at least genes in which at least the partial sequence is known and the function of the expression product of the gene is at least partially understood. Specific human stress genes of interest include those listed in Table 1, infra. A gene is considered to be the same as a gene listed in Table 1 even if it: (a) has a different name or accession number in a gene sequence database, e.g. GENBANK; (b) has at least 90% homology (as determined using the FASTA program with default settings) to the sequence of one of the GENBANK accession numbers listed in Table 1; or (c) belongs to the same gene cluster as defined in NCBI in Unigene database available on the World Wide Web.

The “unique” polynucleotide sequences of each probe spot on the arrays of the subject invention are distinctive or different with respect to every other unique polynucleotide sequence on the arrays that corresponds to a human stress gene, as that term is defined herein. In other words, for at least 80% of the genes on the array, and more usually at least 90% of the genes on the array, any two different unique polynucleotides corresponding to a human stress gene on the array, (i.e. any two unique polynucleotides taken from different, non-identical spots on the array), are not homologous. By not homologous is meant that the sequence identity between the two given unique polynucleotides is less than about 90%, usually less than about 85% and more usually less than about 80% as measured by the FASTA program using default settings. Moreover, each polynucleotide sequence on the array is statistically chosen to ensure that the probability of homology to any sequence of that type is very low. Morever, each unique sequence on the array is statistically chosen to insure that probability of homology to any other known sequence associated with human stress genes is very low, whether or not the other sequence is represented on the array. An important feature of the individual polynucleotide probe compositions of the subject arrays is that they are only a fragment of the entire cDNA of the human stress gene to which they correspond. In other words, for each gene represented on the array, the entire cDNA sequence of the gene is not represented on the array. Instead, the sequence of only a portion or fragment of the entire cDNA is represented on the array by this unique polynucleotide.

The term “polynucleotide probe composition” refers to the nucleic acid composition that makes up each of the probe spots on the array that correspond to a particular human stress gene. Thus, the term ‘polynucleotide probe composition’ includes nucleic acid compositions of unique polynucleotides but excludes control or calibrating polynucleotides (e.g. polynucleotides corresponding to housekeeping genes) which may also be present on the array, as described in greater detail infra. The polynucleotide compositions are made up of single stranded polynucleotides (i.e. polynucleotides that are not hybridized to each other), where all of the polynucleotides in the probe composition may be identical to each other or there may be two or more different polynucleotides (i.e. polynucleotides of different nucleotide sequence) in each probe composition, e.g. where the two different polynucleotides are complementary to each other.

### DESCRIPTION OF THE SPECIFIC EMBODIMENTS

Human stress arrays, as well as methods for their preparation and use, are provided. In the subject human stress arrays, a plurality of polynucleotide probe spots is stably associated with the surface of a solid support. Each different polynucleotide probe spot is made up of a unique polynucleotide that corresponds to a human stress gene of interest. The subject arrays find particular use in gene expression assays of human stress genes. In further describing the subject invention, the human stress arrays themselves are first discussed, followed by a description of methods for their preparation. Next, a review of representative applications in which the subject arrays may be employed is provided.

It is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

Arrays of the Subject Invention-General Description

Array Structure

The arrays of the subject invention have a plurality of polynucleotide probe spots stably (i.e. immobilized such that the probe maintains its position relative to the surface of the array under washing conditions, as described infra) associated with a surface of a solid support. Each probe spot on the array comprises a polynucleotide probe sample or polynucleotide probe composition of known identity, usually of known sequence, as described in greater detail below. The polynucleotide probe spots on the array may be any convenient shape, but will typically be circular, elliptoid, oval, annular, or some other analogously curved shape, where the shape may, in certain embodiments, be a result of the particular method employed to produce the array. The density of the all of the spots on the solid surface, i.e. both probe spots and non-probe spots, e.g. calibration spots, control spots, etc., is at least about 5/cm² and usually at least about 10/cm² but does not exceed about 1000/cm², and in many embodiments does not exceed about 500/cm², where in certain preferred embodiments, the density does not exceed about 400/cm², usually does not exceed about 300/cm², and more usually does not exceed about 60/cm². The spots may be arranged in any convenient pattern across or over the surface of the array, such as in rows and columns so as to form a grid, in a circular pattern, and the like, where generally the pattern of spots will be present in the form of a grid across the surface of the solid support.

In the subject arrays, the spots of the pattern are stably associated with the surface of a solid support, where the support may be a flexible or rigid solid support. By stably associated is meant that the polynucleotides of the spots maintain their position relative to the solid support under hybridization and washing conditions. As such, the polynucleotide members which make up the spots can be non-covalently or covalently stably associated with the support surface. Examples of non-covalent association include non-specific adsorption, binding based on electrostatic (e.g. ion, ion pair interactions), hydrophobic interactions, hydrogen bonding interactions, specific binding through a specific binding pair member covalently attached to the support surface, and the like. Examples of covalent binding include covalent bonds formed between the spot polynucleotides and a functional group present on the surface of the rigid support, e.g. —OH, where the functional group may be naturally occurring or present as a member of an introduced linking group, as described in greater detail below.

The array is present on either a flexible or rigid substrate. By flexible is meant that the support is capable of being bent, folded or similarly manipulated without breakage. Examples of solid materials which are flexible solid supports with respect to the present invention include membranes, flexible plastic films, and the like. By rigid is meant that the support is solid and does not readily bend, i.e. the support is not flexible. As such, the rigid substrates of the subject arrays are sufficient to provide physical support and structure to the polymeric targets present thereon under the assay conditions in which the array is employed, particularly under high throughput handling conditions. Furthermore, when the rigid supports of the subject invention are bent, they are prone to breakage.

The solid supports upon which the subject patterns of spots are present in the subject arrays may take a variety of configurations ranging from simple to complex, depending on the intended use of the array. Thus, the substrate could have an overall slide or plate configuration, such as a rectangular or disc configuration. In many embodiments, the substrate will have a rectangular cross-sectional shape, having a length of from about 10 mm to 200 mm, usually from about 40 to 150 mm and more usually from about 75 to 125 mm and a width of from about 10 mm to 200 mm, usually from about 20 mm to 120 mm and more usually from about 25 to 80 mm, and a thickness of from about 0.01 mm to 5.0 mm, usually from about 0.1 mm to 2 mm and more usually from about 0.2 to 1 mm.

The substrates of the subject arrays may be fabricated from a variety of materials. The materials from which the substrate is fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, is of particular interest in this embodiment. For rigid substrates, specific materials of interest include: glass; plastics, e.g. polytetrafluoroethylene, polypropylene, polystyrene, polycarbonate, and blends thereof, and the like; metals, e.g. gold, platinum, and the like; etc.

The substrates of the subject arrays comprise at least one surface on which the pattern of probe spots is present, where the surface may be smooth or substantially planar, or have irregularities, such as depressions or elevations. The surface on which the pattern of spots is present may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto, e.g. conjugated.

The total number of probe spots on the substrate will vary depending on the number of different polynucleotide probes one wishes to display on the surface, as may be desired depending on the particular application in which the subject arrays are to be employed. Generally, the pattern present on the surface of the array will comprise at least about 10 distinct spots, usually at least about 20 distinct spots, and more usually at least about 50 distinct spots, where the number of spots may be as high as 10,000 or higher, but will usually not exceed about 5,000 distinct spots, and more usually will not exceed about 3,000 distinct spots. In many embodiments, it is preferable to have each distinct probe composition presented in duplicate, i.e. so that there are two spots for each distinct polynucleotide probe composition of the array. In certain embodiments, the number of spots will range from about 100 to 600, usually 200 to 500.

In the arrays of the subject invention (particularly those designed for use in high throughput applications, such as high throughput analysis applications), a single pattern of spots may be present on the array or the array may comprise a plurality of different spot patterns, each pattern being as defined above. When a plurality of different spot patterns are present, the patterns may be identical to each other, such that the array comprises two or more identical spot patterns on its surface, or the spot patterns may be different, e.g. in arrays that have two or more different types of target nucleic acids represented on their surface, e.g an array that has a pattern of spots corresponding to human stress genes and a pattern of spots corresponding to key human genes. Where a plurality of spot patterns are present on the array, the number of different spot patterns is at least 2, usually at least 6, more usually at least 24 or 96, where the number of different patterns will generally not exceed about 384.

Where the array comprises a plurality of spot patterns on its surface, preferably the array comprises a plurality of reaction chambers, wherein each chamber has a bottom surface having associated therewith an pattern of spots and at least one wall, usually a plurality of walls surrounding the bottom surface. Such array configurations and the preparation thereof is further described in U.S. patent application Ser. No. 08/974,298 filed on Nov. 19, 1997, the disclosure of which is herein incorporated by reference. Of particular interest in many embodiments are arrays in which the same pattern of spots in reproduced in 24 or 96 different reaction chambers across the surface of the array.

Within any given pattern of spots on the array, there may be a single spot that corresponds to a given target or a number of different spots that correspond to the same target, where when a plurality of different spots are present that correspond to the same target, the probe compositions of each spot that corresponds to the same target may be identical of different. In other words, a plurality of different targets are represented in the pattern of spots, where each target may correspond to a single spot or a plurality of spots, where the probe composition among the plurality of spots corresponding to the same target may be the same or different. Where a plurality of spots (of the same or different composition) corresponding to the same target is present on the array, the number of spots in this plurality will be at least about 2 and may be as high as 10, but will usually not exceed about 5. The number of different targets represented on the array is at least about 2, usually at least about 10 and more usually at least about 20, where in many embodiments the number of different targets, e.g. genes, represented on the array is at least about 50. The number of different targets represented on the array may be as high as 1000 or higher, but will usually not exceed about 800 and more usually will not exceed about 700. In many preferred embodiments, the number of different targets represented on the array ranges from about 100 to 400, usually from about 200 to 300. A target is considered to be represented on an array if it is able to hybridize to one or more probe compositions on the array.

The amount of polynucleotide present in each spot will be sufficient to provide for adequate hybridization and detection of target nucleic acid during the assay in which the array is employed. Generally, the amount of polynucleotide in each spot will be at least about 0.1 ng, usually at least about 0.5 ng and more usually at least about 1 ng, where the amount may be as high as 1000 ng or higher, but will usually not exceed about 20 ng and more usually will not exceed about 10 ng. The copy number of each polynucleotide in a spot will be sufficient to provide enough hybridization sites for target molecule to yield a detectable signal, and will generally range from about 0.01 fmol to 50 fmol, usually from about 0.05 fmol to 20 fmol and more usually from about 0.1 fmol to 5 fmol. Where the spot has an overall circular dimension, the diameter of the spot will generally range from about 10 to 5,000 μm, usually from about 20 to 2,000 μm and more usually from about 50 to 1000 μm.

A critical feature of the subject arrays is that all of the probe polynucleotide spots of the array correspond to human stress genes of interest, particularly genes that have been identified by those of skill in the art as genes whose products modulate cellular responses to various stressors, including heat shock, irradiation, infection, and treatment with cytotoxic or genotoxic compounds. As such, each polynucleotide probe spot on the array will correspond to a human stress gene of interest. Each probe spot on the array may correspond to a different human stress gene. Alternatively, two or more, usually no more than four, and more usually no more than three, different probe spots may correspond to the same human stress gene, i.e. a human stress gene may be represented by one or a plurality of different probe spots on the array. Furthermore, any given human stress gene may be represented by two or more identical probe spots on the array, e.g. a particular probe spot may be presented on the array once or in duplicate, triplicate, etc, as mentioned above. The number of different human stress genes represented on the array may vary, where in certain embodiments the number of different human stress genes represented on the array will range from about 50 to 1000, usually from about 100 to 700 and more usually from about 100 to 400; while in other embodiments the number will range from about 50 to 2000, usually from about 100 to 1500. A human stress gene is considered to be represented on a given array if a target nucleic acid derived from the human stress gene is able to hybridize to at least one probe spot on the array. Human stress genes that may be represented on the arrays include: genes that express products which are known to modulate the cellular response to stress factors, such as stressors, heat shock, irradiation, infection, and treatment with cytotoxic or genotoxic compounds, and the like. Specific human stress genes that may be represented on the arrays of the subject invention include those listed in Table 1, Table 2 and/or Table 3, infra. In many preferred embodiments, the subject human stress arrays will include at least 20, usually at least 50 and more usually at least 100 of the genes listed in one or more of these tables (i.e. at least 20, usually at least 50 and more usually at least 100 of the genes listed in Table 1 are represented on the array), where in certain preferred embodiments, all of the genes listed in Tables 1, 2 or 3 are present on the array. In one embodiment, the array is made up of polynucleotide probes having a sequence that is identical to and/or complementary to, or at least partially includes, each of the specific gene regions listed in Col. 3 of Table 1.

The average length of the probe polynucleotides on the array is chosen to be of sufficient length to provide a strong and reproducible signal, as well as tight and robust hybridization. In certain embodiments, the average length of the polynucleotides of the array will typically range from about 120 to 1000 nt and usually from about 150 to 800 nt, where in many embodiments, the average length ranges from about 200 to 700 nt, and usually 200 to 600 nt. In yet other embodiments, the length of the probes ranges from 50 to 120 nt, as disclosed in U.S. patent application Ser. No. 09/440,829, the disclosure of which is herein incorporated by reference. The length of each polynucleotide on the array is less than the length of the mRNA to which it corresponds. As such, the polynucleotide represents only a fraction of the full length cDNA to which it corresponds.

The polynucleotide probe compositions that make up each spot on the array will be substantially, usually completely, free of non-nucleic acids, i.e. the probe compositions will not comprise non-nucleic acid biomolecules found in cells, such as proteins, lipids, and polysaccharides. In other words, the oligonucleotide spots of the arrays are substantially, if not entirely, free of non-nucleic acid cellular constituents. By substantially free is meant that the probe composition is at least about 90%, usually at least about 95% and more usually at least about 98% dry weight nucleic acid.

The polynucleotide probes may be nucleic acid, e.g. RNA, DNA, or nucleic acid mimetics, e.g. such as nucleic acids comprising non-naturally occurring heterocyclic nitrogenous bases, peptide-nucleic acids, locked nucleic acids (see Singh & Wengel, Chem. Commun. (1998) 1247-1248); and the like. Nucleic acid mimetics that may be polynucleotide probes on the present arrays include nucleic acids chemically modified from the native phosphodiester structure in order to increase their intracellular stability and binding affinity. A number of such modifications have been described in the literature which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As mentioned above, the subject arrays typically comprise one or more additional spots of polynucleotides which are not human stress genes. Other spots which may be present on the substrate surface include spots comprising genomic DNA, housekeeping genes, negative and positive control genes, and the like. These latter types of spots comprise polynucleotides that are not “unique” as that term is defined and used herein, i.e. they are “common.” In other words, they are calibrating or control genes whose function is not to tell whether a particular “key” human stress gene of interest is expressed, i.e. whether a particular human stress gene is expressed in a particular sample, but rather to provide other useful information, such as background or basal level of expression, and the like. For example, spots comprising genomic DNA may be provided in the array, where such spots may serve as orientation marks. Spots comprising plasmid and bacteriophage genes, genes from the same or another species which are not expressed and do not cross hybridize with the cDNA target, and the like, may be present and serve as negative controls. Specific negative controls of interest include: M13 mp18(+) strand DNA, lambda DNA and pUC18. In addition, spots comprising housekeeping genes and other control genes from the same or another species may be present, which spots serve in the normalization of mRNA abundance and standardization of hybridization signal intensity in the sample assayed with the array. Specific housekeeping genes of interest include: ubiquitin, phospholipase A2, hypoxanthine-guanine phosphoribosyl transferase, glyceraldehyde 3-phosphate dehydrogenase, tubulina alpha, HLA class I histocompatibility antigen, C-4 alpha chain, beta-actin, 23 kDa highly basic protein and ribosomal protein S9.

Polynucleotide Probes of the Arrays

Each probe spot of the pattern present on the surface of the substrate is made up of a unique polynucleotide probe composition. By “polynucleotide probe composition” is meant a collection or population of single stranded polynucleotides capable of participating in a hybridization event under appropriate hybridization conditions, where each of the individual polynucleotides may be the same—have the same nucleotide sequence—or have different sequences, for example the probe composition may consist of 2 different single stranded polynucleotides that are complementary to each other (i.e. the two different polynucleotides in the spot are complementary but physically separated so as to be single stranded, i.e. not hybridized to each other). In many embodiments, the probe compositions will comprise two complementary, single stranded polynucleotides. In the polynucleotide probe compositions, the sequence of the polynucleotides are chosen so that each distinct unique polynucleotide does not cross-hybridize with any other distinct unique polynucleotide of another probe spot on the array, i.e. the polynucleotide of any other polynucleotide composition that corresponds to a human stress gene. As such, the nucleotide sequence of each unique polynucleotide of a probe composition will have less than 90% homology, usually less than 85% homology, and more usually less than 80% homology with any other different polynucleotide of a probe composition of the array, where homology is determined by sequence analysis comparison using the FASTA program using default settings. The sequence of unique polynucleotides in the probe compositions are not conserved sequences found in a number of different genes (at least two), where a conserved sequence is defined as a stretch of from about 40 to 200 nucleotides which have at least about 90% sequence identity, where sequence identity is measured as above. The polynucleotide will generally be a deoxyribonucleic acid having a length of from about 120 to 1000, usually from 120 to 700 nt, and more usually 200 to 600 nt in certain embodiments; while in other embodiments the length will range from 50 to 120 nt, as described above. The polynucleotide will not cross-hybridize with any other polynucleotide on the array under standard hybridization conditions and usually stringent hybridization conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art and may also be employed to identify nucleic acids of this particular embodiment of the invention. Again, the length of the polynucleotide will be shorter than the mRNA to which it corresponds.

Array Preparation

The subject arrays can be prepared using any convenient means. One means of preparing the subject arrays is to first synthesize the polynucleotides for each spot and then deposit the polynucleotides as a spot on the support surface. The polynucleotides may be prepared using any convenient methodology, such as automated solid phase synthesis protocols, restriction digestion of a gene fragment insert cloned into a vector, preparative PCR and like, where preparative PCR or enzymatic synthesis is preferred in view of the length and the large number of polynucleotides that must be generated for each array. In the case of automated solid phase synthesis, each polynucleotide can be represented by several overlapping or non-overlapping oligonucleotides from 10 to 100 nucleotides in length, which cover all or a partial sequence of a gene or polynucleotide. See U.S. patent application Ser. No. 60/104,179, the disclosure of which is herein incorporated by reference.

For preparative PCR, primers flanking either side of the portion of the gene of interest will be employed to produce amplified copy numbers of the portion of interest. Methods of performing preparative PCR are well known in the art, as summarized in PCR, Essential Techniques (Ed. J. F. Burke, John Wiley & Sons)(1996). Alternatively, if a gene fragment of interest is cloned into a vector, vector primers can be used to amplify the gene fragment of interest to produce the polynucleotide.

In determining the portion of the gene to be amplified and subsequently placed on the array, regions of the gene having a sequence unique to that gene should preferably be amplified. Different methods may be employed to choose the specific region of the gene to be amplified. Thus, one can use a random approach based on availability of a gene of interest. However, instead of using a random approach which is based on availability of a gene of interest, a rational design approach may also be employed to choose the optimal sequence for the hybridization array. Preferably, the region of the gene that is selected and amplified is chosen based on the following criteria. First, the sequence that is chosen should yield a polynucleotide that does not cross-hybridize with any other polynucleotide that is present on the array. Second, the sequence should be chosen such that the polynucleotide has a low probability of cross-hybridizing with a polynucleotide having a nucleotide sequence found in any other gene, whether or not the gene is to be represented on the array. As such, sequences that are avoided include those found in: highly expressed gene products, structural RNAs, repeated sequences found in the sample to be tested with the array and sequences found in vectors. A further consideration is to select sequences which provide for minimal or no secondary structure, structure which allows for optimal hybridization but low non-specific binding, equal or similar thermal stabilities, and optimal hybridization characteristics.

The prepared polynucleotides may be spotted on the support using any convenient methodology, including manual techniques, e.g. by micro pipette, ink jet, pins, etc., and automated protocols. See U.S. Pat. No. 5,770,151 and WO 95/35505, the disclosures of which are herein incorporated by reference, for discussions of representative ways of spotting polynucleotides on a support. Of particular interest is the use of an automated spotting device, such as the Beckman Biomek 2000 (Beckman Instruments). As mentioned above, the polynucleotide probe compositions that are spotted onto the array surface are made up of single stranded polynucleotides, where all the polynucleotides may be identical to each other or a population of complementary polynucleotides may be present in each spot.

Methods of Using the Subject Arrays

The subject arrays find use in a variety of different applications in which one is interested in detecting the occurrence of one or more binding events between target nucleic acids and probes on the array and then relating the occurrence of the binding event(s) to the presence of a target(s) in a sample, i.e. the expression of a particular human stress gene in a sample. In general, the device will be contacted with the sample suspected of containing the target human stress gene under conditions sufficient for binding of any target present in the sample to a complementary polynucleotide present on the array. Generally, the sample will be a fluid sample and contact will be achieved by introduction of an appropriate volume of the fluid sample onto the array surface, where introduction can via inlet port, deposition, dipping the array into a fluid sample, and the like.

Generation of Labeled Target

Targets may be generated by methods known in the art, mRNA can be labeled and used directly as a target, or converted to a labeled cDNA target. Generally, such methods include the use of oligonucleotide primers. Primers that may be employed include oligo dT, random primers, e.g. random hexamers and gene specific primers, as described in U.S. patent application Ser. No. 08/859,998, the disclosure of which is herein incorporated by reference. Where gene specific primers are employed, the gene specific primers are preferably those primers that correspond to the different polynucleotide spots on the array. Thus, one will preferably employ gene specific primers for each different polynucleotide that is present on the array, so that if the gene is expressed in the particular cell or tissue being analyzed, labeled target will be generated from the sample for that gene. In this manner, if a particular human stress gene present on the array is expressed in a particular sample, the appropriate target will be generated and subsequently identified.

A variety of different protocols may be used to generate the labeled target nucleic acids, as is known in the art, where such methods typically rely on the enzymatic generation of the labeled target using the initial primer. Labeled primers can be employed to generate the labeled target. Alternatively, label can be incorporated during first strand synthesis or subsequent synthesis, labeling or amplification steps in order to produce labeled target. Representative methods of producing labeled target are disclosed in U.S. patent application Ser. No. 08/859,998, the disclosure of which is herein incorporated by reference. Alternatively, the label can be introduced by chemical cDNA synthesis.

Hybridization and Detection

As mentioned above, following preparation of the target nucleic acid from the tissue or cell of interest, the labeled target nucleic acid is then contacted with the array under hybridization conditions, where such conditions can be adjusted, as desired, to provide for an optimum level of specificity in view of the particular assay being performed. Suitable hybridization conditions are well known to those of skill in the art and reviewed in Maniatis et al, supra and WO 95/21944, e.g. stringent conditions (e.g. at 50° C. or higher and 0.1×SSC (15 mM sodium chloride/01.5 mM sodium citrate). In analyzing the differences in the population of labeled target nucleic acids generated from two or more physiological sources using the arrays described above, each population of labeled target nucleic acids are separately contacted to identical probe arrays or together to the same array under conditions of hybridization, preferably under stringent hybridization conditions, such that labeled target nucleic acids hybridize to complementary probes on the substrate surface.

Where all of the target sequences comprise the same label, different arrays will be employed for each physiological source (where different could include using the same array at different times). Alternatively, where the labels of the targets are different and distinguishable for each of the different physiological sources being assayed, the opportunity arises to use the same array at the same time for each of the different target populations. Examples of distinguishable labels are well known in the art and include: two or more different emission wavelength fluorescent dyes, like Cy3 and Cy5, two or more isotopes with different energy of emission, like ³²P and ³³P, light scattering particles with different scattering spectra, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Following hybridization, non-hybridized labeled nucleic acid is removed from the support surface conveniently by washing, generating a pattern of hybridized nucleic acid on the substrate surface. A variety of wash solutions are known to those of skill in the art and may be used.

The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, colorimetric measurement, light emission measurement, light scattering and the like.

Following detection or visualization, the hybridization patterns may be compared to identify differences between the patterns. Where arrays in which each of the different probes corresponds to a known gene are employed, any discrepancies can be related to a differential expression of a particular gene in the physiological sources being compared.

Utility

The subject methods find use in, among other applications, in differential human stress gene expression assays. Thus, one may use the subject methods in the differential expression analysis of human stress genes in: (a) diseased and normal tissue, e.g. neoplastic and normal tissue, (b) different tissue or tissue types; (c) developmental stage; (d) response to external or internal stimulus; (e) response to treatment; and the like. The subject arrays therefore find use in broad scale expression screening for research into the role of human stress genes in normal and diseased conditions, as well as in high throughput drug screening and drug discovery and research, such as the effect of a particular active agent on the expression pattern of genes in a particular cell, where such information can be used to reveal drug toxicity, carcinogenicity, etc., environmental monitoring, disease research and the like.

Kits

Also provided are kits for performing analyte binding assays using the subject devices, where kits for carrying out differential gene expression analysis assays are preferred. Such kits according to the subject invention will at least comprise a human stress array according to the subject invention. The kits may further comprise one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, or other post synthesis labeling reagent, such as chemically active derivatives of fluorescent dyes, biotin, digoxigenin, or strept/avidin-label conjugate or antibody-label conjugate, enzymes, such as reverse transcriptases, DNA polymerases, and the like, various buffer mediums, e.g. hybridization and washing buffers, labeled target purification reagents and components, like spin columns, etc., signal generation and detection reagents, e.g. streptavidin-alkaline phosphatase conjugate, chemifluorescent or chemiluminescent substrate, and the like.

The following examples are offered by way of illustration and not by way of limitation.

### EXPERIMENTAL

### EXAMPLE 1

### Generation of Human Stress cDNA Array

236 cDNA fragments corresponding to 236 different human stress genes as listed in Table 1 were amplified from quick-clone cDNA (CLONTECH) in 236 separate test tubes using a combination of sense and antisense gene-specific primers capable of amplifying the specific gene fragments of interest as specified in Table 1. Amplification was conducted in a 100-μl volume containing 2 μl of mixture of 10 Quick-clone cDNA from placenta, brain, liver, lung, leukocytes, spleen, skeletal muscle, testis, kidney and ovary (CLONTECH), 40 mM Tricine-KOH (pH 9.2 at 22° C.), 3.5 mM Mg(OAc)₂, 10 mM KOAc, 75 μg/ml BSA, 200 μM of each dATP, dGTP, dCTP and dTTP, 0.2 μM of each sense and antisense gene-specific primers and 2 μl of KlenTaq Polymerase mix. Temperature parameters of the PCR reactions were as follows: 1 min at 95° C. followed by 20-35 cycles of 95° C. for 15 sec and 68° C. for 2 min; followed by a 10-min final extension at 68° C. PCR products were examined on 1.2% agarose/EtBr gels in 1×TBE buffer. As a DNA size marker a 1 Kb DNA Ladder was used. ds cDNA was then precipitated by addition of a half volume of 4M ammonium acetate (about 35 μl) and 3.7 volumes of 95% ethanol (about 260 μl). After vortexing, the tube was immediately centrifuged at 14,000 r.p.m. in a microcentrifuge for 20 min. The pellet was washed with 80% ethanol without vortexing, centrifuged as above for 10 min, air dried, and dissolved in 10 μl of deionized water. Yield of ds cDNA after the amplification step was about 5 μg. The ds cDNA fragments for all 236 genes were cloned into pAtlas 1A-cloning vector (Clontech) using blunt end ligation by T4 DNA polymerase and identity of the clones was confirmed by sequence analysis. The ds cDNA inserts with the sequence corresponding 236 genes were amplified by PCR using a combination of antisense and sense gene-specific primers, as described above. The ds cDNA was denatured by adding 1 μl of 10×denaturing solution (1 M NaOH, 10 mM EDTA) and incubating at 65° C. for 20 min. All cDNA probes were transferred in 384-well plate and loaded on positively charged nylon membrane (Schleher & Schull) using 384 pin tool and Biomek 2000 (Beckman) robot.

The resultant array has 236 different human stress genes represented on it. The specific human stress genes represented on the array are listed in Table 1. Also provided in Table 1 is the specific region of each gene that is represented on the array. See Col. 3 of Table 1. Thus, Table 1 also provides the sequence for each polynucleotide probe on the array. For example, the array comprises a polynucleotide probe to MDM2 protein, where the probe has a DNA sequence that is either complementary or identical to the sequence of the mRNA from the 920-1232 nucleotide of the sense strand counting from the 5′ end of the mRNA sequence, where the entire sequence has been deposited in GENBANK under accession nos. Z12020 and M92424 and is therefore readily available.

TABLE 1

| | | FRAGMENT |
|---|---|---|
| | | LENGTH, bp |
| GENE NAME | GENBANK # | (start-end) |
| MDM2 PROTEIN (p53-associated gene) + | Z12020; M92424 | 920-1232 |
| MDM2-A + MDM2-C | | |
| p53 cellular tumor antigen | M14694; M14695 | 690-964 |
| basic transcription factor 62 kDa subunit | M95809 | 1449-1831 |
| basic transcription factor 44 kDa subunit | Z30094 | 606-843 |
| DNA-binding protein PO-GA | L14922 | 3196-3413 |
| TCF5 [HEAT SHOCK FACTOR PROTEIN | M64673 | 294-572 |
| 1] CCAAT enhancer-binding protein beta | | |
| [HG99] | | |
| recA-like protein HsRad51; DNA REPAIR | D13804 | 867-1159 |
| PROTEIN RAD51 HOMOLOG | | |
| excision repair protein ERCC6 | L0479 1 | 1772-2194 |
| MAP kinase kinase; DUAL SPECIFICITY | L05624 | 842-1217 |
| MITOGEN-ACTIVATED PROTEIN | | |
| KINASE KINASE 1 (EC 2.7.1.-); ERK | | |
| ACTIVATOR KINASE 1); MAPK/ERK | | |
| KINASE 1 (MEK1) | | |
| replication factor C 36-kDa subunit | L07540 | 708-1051 |
| replication factor C 38-kDa subunit | L07541 | 438-762 |
| ERCC5 excision repair protein; DNA- | L20046; X69978 | 1374-1638 |
| REPAIR PROTEIN COMPLEMENTING | | |
| XP-G CELLS (XERODERMA | | |
| PIGMENTOSUM GROUP G | | |
| COMPLEMENTING PROTEIN) | | |
| Rad | L24564 | 489-780 |
| Ku (p70/p80) subunit; ATP-DEPENDENT | M30938 | 2340-2764 |
| DNA HELICASE II, 86-kDa SUBUNIT; | | |
| LUPUS KU AUTOANTIGEN PROTEIN | | |
| P86; THYROID-LUPUS AUTOANTIGEN | | |
| (TLAA); CTC BOX BINDING FACTOR 85- | | |
| kDa SUBUNIT (CTCBF) (CTC85); | | |
| NUCLEAR FACTOR IV (KU80) (XRCC5) | | |
| cyclic AMP response element-binding protein | M31630 | 316-636 |
| (HB 16) 3′ end | | |
| DNA repair helicase (ERCC3); DNA- | M31899 | 2109-2466 |
| REPAIR PROTEIN COMPLEMENTING | | |
| XP-B CELLS; XERODERMA | | |
| PIGMENTOSUM GROUP B | | |
| COMPLEMENTING PROTEIN; BASAL | | |
| TRANSCRIPTION FACTOR 2 89-kDa | | |
| SUBUNIT (BTF2-p89); TFIIH 89-kDa | | |
| SUBUNIT | | |
| Ku protein subunit; ATP-DEPENDENT DNA | M32865; S38729 | 1729-1974 |
| HELICASE II 70-kDa SUBUNIT; LUPUS | | |
| KU AUTOANTIGEN PROTEIN P70; | | |
| THYROID-LUPUS AUTO-ANTIGEN | | |
| (TLAA); CTC BOX BINDING FACTOR 75- | | |
| kDa SUBUNIT (CTCBF) (CTC75) (XRCC6) | | |
| active transcription factor CREB | M34356 | 433-780 |
| DNA-repair protein (XRCC1) | M36089 | 1226-1539 |
| replication protein A 70-kDa subunit (RP-A) | M63488 | 1498-1838 |
| (RE-A); SINGLE-STRANDED DNA- | | |
| BINDING PROTEIN | | |
| HHR6A (yeast RAD 6 homologue) | M74524 | 175-433 |
| replication factor C 40-kDa subunit (A1) | M87338 | 882-1286 |
| replication factor C 37-kDa subunit | M87339 | 98-355 |
| melanoma differentiation associated protein 6 | U09579; L25610 | 1745-2063 |
| (mda-6); CYCLIN-DEPENDENT KINASE | | |
| INHIBITOR 1; CDK-INTERACTING | | |
| PROTEIN 1 (CIP1) (WAF1) (CDKN1A) | | |
| (CDKN1) (SDI1) (PIC1) (CAP20) | | |
| DNA polymerase alpha-subunit | X06745 | 3721-4093 |
| single-stranded DNA-binding protein pur- | M96684 | 563-855 |
| alpha | | |
| DNA topoisomerase I | J03250 | 2388-2796 |
| DNA topoisomerase II alpha | J04088 | 2459-2883 |
| 6-O-methylguanine-DNA methyltransferase | M29971 | 241-546 |
| (MGMT); METHYLATED-DNA-- | | |
| PROTEIN-CYSTEINE | | |
| METHYLTRANSFERASE | | |
| DNA excision repair protein ERCC2 5′ end; | X52221; HT1175 | 1520-1821 |
| DNA-REPMR PROTEIN | | |
| COMPLEMENTING XP-D CELLS; | | |
| XERODERMA PIGMENTOSUM GROUP D | | |
| COMPLEMENTING PROTEIN | | |
| glutathione reductase | HT1483 | 719-1057 |
| glutathione 5-transferase 12 | HT1790 | 72-420 |
| DNA excision repair protein ERCC1 | M13194; HT1848 | 625-938 |
| glutathione 5-transferase M1 | HT2041 | 504-906 |
| glutathione 5-transferase pi | HT2042 | 203-511 |
| glutathione S-transferase A1 | HT2168 | 257-583 |
| glutathione peroxidase | HT2859 | 454-745 |
| superoxide dismutase 1 cytosolic | K00065; HT3218 | 198-496 |
| DNA mismatch repair protein hmlh 1 | U07418; HT3337 | 1765-2020 |
| xeroderma pigmentosum group C repair | HT4209 | 582-885 |
| complementing protein p58/HHR23B | | |
| xeroderma pigmentosum group C repair | D21235; HT4247 | 355-632 |
| complementing protein HHR23A; UV | | |
| EXCISION REPAIR PROTEIN PROTEIN | | |
| RAD23 | | |
| glutathione 5-transferase T1 | HT4547 | 617-914 |
| heat-shock protein 40 | D49547 | 1400-1782 |
| STRESS-ACTIVATED protein kinase | L26318 | 952-1263 |
| (JNK1) (EC 2.7.1.-); C-JUN N-TERMINAL | | |
| KINASE 1 (JNK-46) | | |
| p38 mitogen activated protein (MAP) kinase; | L35253; L35263 | 925-1204 |
| CYTOKINE SUPPRESSIVE ANTI- | | |
| INFLAMMATORY DRUG BINDING | | |
| PROTEIN; CSAID BINDING PROTEIN) | | |
| (CSBP); MAX-INTERACTING PROTEIN 2 | | |
| (MAP KINASE MXI2) | | |
| chaperonin (HSP6O) | M34664 | 533-839 |
| growth arrest and DNA-damage-inducible | M60974 | 526-886 |
| protein (gadd45) | | |
| extracellular signal-regulated kinase 2 | M84489 | 1241-1522 |
| GADD153 = growth arrest and DNA-damage- | S40706; S62138 | 480-789 |
| inducible | | |
| DNA-DEPENDENT PROTEIN KINASE | U35835; U47077 | 2250-2680 |
| (DNA-PK) + DNA-PK CATALYTIC | | |
| SUBUNIT (XRCC7) | | |
| breast cancer susceptibility (BRCA2) | U43746 | 10056-10346 |
| heat shock protein hsp86 | X07270 | 380-577 |
| heat shock protein HSP27 | X54079 | 423-683 |
| VIMENTIN | X56134 [M14144] | 1164-1604 |
| EXTRACELLULAR SIGNAL- | X60188 | 754-1094 |
| REGULATED KINASE 1 (ERK1) protein | | |
| serine/threonine kinase (EC 2.7.1.-); | | |
| INSULIN- STIMULATED MAP2 KINASE; | | |
| MAP KINASE 1 (MAPK 1) (P44-ERK1) | | |
| (ERT2); MICROmBULE-ASSOCIATED | | |
| PROTEIN-2 KINASE (P44-MAPK) | | |
| EXTRACELLULAR SIGNAL- | X80692 | 806-1267 |
| REGULATED KINASE 3 (EC 2.7.1.-) | | |
| (ERK3); MAP KINASE ISOFORM P97 | | |
| (P97-MAPK) | | |
| sTRESS-ACTIVATED PROTEIN KINASE | L31951 | 638-1000 |
| JNK2 (EC 2.7.1.-); C-JUN N-TERMINAL | | |
| KINASE 2 (JNK-55) | | |
| STRESS-ACTIVATED PROTEIN KINASE | U34819; U07620 | 1018-1413 |
| JNK3 (EC 2.7.1.-); C-JUN N-TERMINAL | | |
| KINASE 3 (JNK3); MAP KINASE P49 3F12 | | |
| heat shock protein (HSP 70) | M11717 | 1962-2225 |
| DNA damage repair and recombination | U12134 | 1528-1733 |
| protein RAD52 | | |
| ataxia telangiectasia (ATM) | U33841 | 8938-9135 |
| DUAL-SPECIFICITY MITOGEN- | U39657 | 1060-1389 |
| ACTIVATED PROTEIN KINASE KINASE | | |
| 6 (EC 2.7.1.-); MAP KINASE KINASE 6) | | |
| (MAPKK 6); MAPKIERK KINASE 6 | | |
| (SAPKK3) | | |
| Rad50 (Rad50) | U63139 | 5117-5435 |
| DNA ligase IV; | X83441 | 2787-3074 |
| POLYDEOXYRIBONUCLEOTIDE | | |
| SYNTHASE (ATP) | | |
| DNA ligase III; | X84740 | 2460-2780 |
| POLYDEOXYRIBONUCLEOTIDE | | |
| SYNTHASE (ATP) | | |
| MUSCLE-SPECIFIC DNASE I-LIKE | X90392; L40817; U06846 | 2038-2427 |
| (DNase X) | | |
| cdc42 homolog (G25K) [brain isoform + | M35543; [M57298] | 295-494 |
| placental isoform] | | |
| p21-activated protein kinase (Pak2) | U24153 | 335-671 |
| EXTRACELLULAR SIGNAL- | U25278 | 1010-1267 |
| REGULATED KINASE 5 (EC 2.7.1.-) | | |
| (ERK5) (ERK4) (BMK1 KINASE) | | |
| MITOGEN-ACTIVATED PROTEIN | U53442 | 1119-1356 |
| KINASE P38 BETA (EC 2.7.1.-) (MAP | | |
| KINASE P38 BETA) | | |
| FKBP-RAPAMYSIN ASSOCIATED | L34075 | 6750-7088 |
| PROTEIN (FRAP) | | |
| EXTRACELLULAR SIGNAL- | X59727 | 2678-2994 |
| REGULATED KINASE 4 (EC 2.7.1.-) | | |
| (ERK4) (MAP KINASE 1SOFORM P63) | | |
| (P63-MAPK). | | |
| EXTRACELLULAR SIGNAL- | X79483 | 530-831 |
| REGULATED KINASE 6 (EC 2.7.1.-) | | |
| (ERK6) (ERK5) | | |
| PCNA (CYCLIN) | M15796; [J04718] | 157-436 |
| MEKKINASE3 | U78876 | 1195-1453 |
| BCL-2 BINDING ATHANOGENE-1 (BAG- | 583171; [Z35491] | 511-830 |
| 1) (GLUCOCORTICOID RECEPTOR- | | |
| ASSOCIATED PROTEIN RAP46). | | |
| C-fos | K00650 | 2949-3181 |
| DUAL SPECIFICITY MITOGEN- | L11285 | 344-687 |
| ACTIVATED PROTEIN KLIINASE KINASE | | |
| 2 (EC 2.7.1.-) (MAP KINASE KINASE 2) | | |
| (MAPKK 2) (ERK ACTIVATOR KINASE | | |
| 2) (MAPKJERK KIINASE 2) (MEK2). | | |
| DUAL SPECIFICITY MITOGEN- | U25265 | 603-845 |
| ACTIVATED PROTEIN KINASE KINASE | | |
| 5 (EC 2.7.1.-) (MAP KINASE KTNASE 5) | | |
| (MAPKK 5) (MAPK/ERK KINASE 5). | | |
| glutathione-S-transferase homolog | U90313 | 97-345 |
| DNA MISMATCH REPAIR PROTEIN | U04045; [L47583] | 2152-2365 |
| MSH2 | | |
| DNA MISMATCH REPAIR PROTEIN | U54777 | 2200-2475 |
| MSH6 (mutS - ALPHA 160 KD SUBUNIT) | | |
| (G/T MISMATCH BINDING PROTEIN) | | |
| (GTBP) (GTMBP) (P160) | | |
| NUCLEOSIDE DIPHOSPHATE KINASE A | X17620 | 245-492 |
| (EC 2.7.4.6) (NDK A) (NDP KINASE A) | | |
| (TUMOR METASTATIC PROCESS- | | |
| ASSOCIATED PROTEIN) (METASTASIS | | |
| INHIBITION FACTOR NM23) (NM23-H1). | | |
| NCK, ASH AND PHOSHPHOLIPASE C | AB005216 | 1003-1274 |
| GAMMA-BINDING PROTIEN | | |
| NAP4(AB0052 16) | | |
| DNA POLYMERASE BETA (DPOB) | D29013 | 812-1103 |
| DNA TOPOISOMERASE II, BETA | X68060 | 3597-3877 |
| (TOP2B) | | |
| DIMETHYLANILINE | Z11737 | 1364-1641 |
| MONOOXYGENASE (N-OXIDE | | |
| FORMING) 4 (EC 1.14.13.8) (HEPATIC | | |
| FLAVIN-CONTAINING | | |
| MONOOXYGENASE 4) (FMO 4) | | |
| (DIMETHYLANILINE OXIDASE 4) | | |
| DNA-REPAIR PROTEIN | L77890 | 1998-2269 |
| COMPLEMENTING XP-F CELLS | | |
| (XERODERMA PIGMENTOSUM GROUP | | |
| F COMPLEMENTING PROTEIN) (DNA | | |
| EXCISION REPAIR PROTEIN ERCC-4) | | |
| 78 KD GLUCOSE REGULATED PROTEIN | M19645 | 675-977 |
| PRECURSOR (GRP 78) | | |
| (IMMUNOGLOBULIN HEAVY CHAIN | | |
| BINDING PROTEIN) (BIP) | | |
| V(D)J RECOMBINATION ACTIVATING | M94633 | 1296-1644 |
| PROTEIN 2 (RAG2) (RAG-2) | | |
| DNA MISMATCH REPAIR PROTEIN | U13695 | 183-419 |
| PMSl (PMSI PROTEIN HOMOLOG 1) | | |
| DNA MISMATCH REPAIR PROTEIN | U13696 | 2354-2579 |
| PMS2 (PMS 1 PROTEIN HOMOLOG 2) | | |
| REPLICATION PROTEIN A 30 KD | U24186 | 135-424 |
| SUBUNIT (RP-A) (RE-A) (REPLICATION | | |
| FACTOR-A PROTEIN 4) | | |
| mutY HOMOLOG (HMYH) | U63329 | 124-370 |
| BETA CRYSTALLIN A4 (H5U59057). | U59057 | 229-570 |
| T-COMPLEX PROTEIN 1, EPSILON | D43950 | 326-610 |
| SUBUNIT (TCP- 1 -EPSILON)(CCT- | | |
| EPSILON) (HUMKGI DD) | | |
| BETA CRYSTALLIN BI (CRYBB1) | U35340 | 229-481 |
| (H5U35340). | | |
| BETA CRYSTALLIN B2 (BP) | L10035 | 270-593 |
| (HUMCRYB2B). | | |
| BETA CRYSTALLIN B3 (9CRYBB3 OR | U71216 | 163-375 |
| CRYB3) (H5U71216). | | |
| B LYMPHOCYTE GERMINAL CENTER | U07349 | 137-398 |
| KINASE (HSU07349) | | |
| CYTOCHROME P450 IIA6 (EC 1.14.14.1) | M33318; [X13930; | 1007-1332 |
| | X13897]; | |
| (COUMARIN 7-HYDROXYLASE) (11A3) | M33317 | |
| (P450(I)) (PHENOBARIBITAL- | | |
| iNDUCIBLE) | | |
| CYTOCHROME P450 11A7 (EC 1.14.14.1) | | |
| (P450-IIA4) | | |
| CYTOCHROME P450 11C9 (EC 1.14.14.1) | M21940; M15331; | 627-917 |
| (P450 PB-I) (P450 MP-4) (S- | [M21939]M61858; | |
| | [L07093]; | |
| MEPHENYTOIN 4-HYDROXYLASE) + | M61853; M61854 | |
| CYTOCHROME P450 IIC10 (EC 1.14. 14.1) | | |
| (P450 MP-8) (8-MEPHENYTOIN 4- | | |
| ITYDROXYLASE) (FRAGMENT) | | |
| INTERFERON-INDUCIBLE RNA- | M35663; [U50648] | 758-984 |
| DEPENT)ENT PROTEiN KINASE (P68 | | |
| KINASE) | | |
| 10 KD HEAT SHOCK PROTEIN, | U07550 | 101-385 |
| MITOCHONDRIAL (HSP 10) (10 KD | | |
| CHAPERONIN) (CPN 10). | | |
| HEAT SHOCK PROTEIN 27 (heart) | U15590 | 502-768 |
| PROTEIN DISULFIDE ISOMERASE PS | D49489 | 1118-1397 |
| PRECURSOR (EC 5.3.4.1) (HUMPS). | | |
| HEAT SHOCK PROTEIN HSP 90-BETA | M16660 | 135-386 |
| (HSP 84) (HSP 90) | | |
| HEAT SHOCK 70 KD PROTEIN 6 (HEAT | X51757; M11236 | 1814-2229 |
| SHOCK 70 KD PROTEIN B′). HEAT | | |
| SHOCK 70 KD PROTEIN 7 (HEAT SHOCK | | |
| 70 KD PROTEIN B) (FRAGMENT). | | |
| HEAT SHOCK COGNATE 71 KD | Y00371 | 526-754 |
| PROTEIN. | | |
| HEAT SHOCK-RELATED 70 KD | L26336 | 1246-1548 |
| PROTEIN 2 (HEAT SHOCK 70 KD | | |
| PROTEIN 2). | | |
| CYTOCHROME P450 IIE1 (EC 1.14.14.1) | J02625 | 674-961 |
| (P450-3) (ETHANOL INDUCIBLE) CYP2E1 | | |
| CYTOCHROME P450 IIF1 (EC 1.14.14.1) | J02906 | 442-719 |
| CYP2F1. | | |
| UDP-GLUCURONOSYLTRANSFERASE | J04093 | 444-677 |
| 1-6 PRECURSOR, MICROSOMAL (EC | | |
| 2.4.1.17) (UDPGT) (UGT-IF) (UGT1*6) | | |
| (UGTI-06) (UGT 1.6) (UGT 1A6) (UGT1F) | | |
| (PHENOL SPECIFIC) UGTI OR GNTI. | | |
| GLUTATH10NE 5-TRANSFERASE MU 3 | 305459 | 252-479 |
| (EC 2.5.1.18) (GSTM3-3) (CLASS-MU) | | |
| GSTM3 OR GST5. | | |
| CYTOCHROME P450 IAl (EC 1.14.14.1) | K03191 | 787-1029 |
| (P450-P1) (P450 FORM 6) (P450-C) (TCDD- | | |
| INDUCIBLE). | | |
| PEROXISOME PROLIFERATOR | L02932 | 221-475 |
| ACTIVATED RECEPTOR ALPHA (PPAR- | | |
| ALPHA) PPARA OR PPAR | | |
| PROTEIN DISULFIDE ISOMERASE- | D49490 | 1233-1548 |
| RELATED PROTEIN PRECURSOR (EC | | |
| 5.3.4.1) (PDIR) (HUMPDIR). | | |
| SOLUBLE EPOXIDE HYDROLASE (SEH) | L05779 | 913-1202 |
| (EC 3.3.2.3) (EPOXIDE HYDRATASE) | | |
| (CYTOSOLIC EPOXIDE HYDROLASE) | | |
| (CEH) EPHX2. | | |
| LIVER CARIBOXYLESTERASE | L07765 | 1100-1342 |
| PRECURSOR (EC 3.1.1.1) (ACYL | | |
| COENZYME A:CHOLESTEROL | | |
| ACYLTRANSFERASE) (ACAT) | | |
| (MONOCYTE/MACROPHAGE SERINE | | |
| ESTERASE) (HMSE) CES2. | | |
| SERUM | L48513 | 790-1071 |
| PARAOXONASE/ARYLESTERASE 2 (EC | | |
| 3.1.1.2) (EC 3.1.8.1) (PON 2) (SERUM | | |
| ARYLDIAKYLPHOSPHATASE 2) (A- | | |
| ESTERASE 2) (AROMATIC ESTERASE 2) | | |
| PON2. | | |
| SERUM | L48516 | 638-947 |
| PARAOXONASE/ARYLESTERASE 3 (EC | | |
| 3.1.1.2) (EC 3.1.81) (PON 3) (SERUM | | |
| ARYLDIAKYLPHOSPHATASE 3) (A- | | |
| ESTERASE 3) (AROMATIC ESTERASE 3) | | |
| (FRAGMENT) PON3. | | |
| CYTOCHROME P450 XXIB (EC | M12792; [M23280] | 603-885 |
| 1.14.99.10) (STEROID 21- | | |
| HYDROXYLASE) (P450-C21B) CYP2IB | | |
| ORCYP21 ORCYP21A2. | | |
| CYTOCHROME P450 XIA1, | M14565 | 1076-1391 |
| MITOCHONDRIAL PRECURSOR (EC | | |
| 1.14.15.6) (P450(SCC)) (CHOLESTEROL | | |
| SIDE-CHAIN CLEAVAGE ENZYME) | | |
| (CHOLESTEROL DESMOLASE) | | |
| CYPi lAl. | | |
| CYTOCHROME P450 11D6 (EC 1.14.14.1) | M20403 | 899-1169 |
| (P450-DB1) (DEBRISOQUINE 4- | | |
| HYDROXYLASE) CYP2D6. | | |
| UDP-GLUCURONOSYLTRANSFERASE | M57899 | 788-1019 |
| 1-1 PRECURSOR, MICROSOMAL (EC | | |
| 2.4.1.17) (UDPGT) (UGT- IA) (UGT1*1) | | |
| (UGT1-01) (UGTI.1) (UGT1A1) | | |
| (BILIRUBIN SPECIFIC ISOZYME 1) | | |
| (UGTIA) (IIUG-BRi) UGTI OR GNT1. | | |
| UDP-GLUCURONOSYLTRANSFERASE | M57951 | 393-661 |
| 1-4 PRECURSOR, MICROSOMAL (EC | | |
| 2.4.1.17) (UDPGT) (UGT- ID) (UGT 1*4) | | |
| (UGT 1-04) (UGT1 .4) (UGTIA4) (UGT1D) | | |
| (BILIRUBIN SPECIFIC ISOZYME 2) | | |
| (HUG-BR2) UGT1 OR GNT1. | | |
| STE20-LIKE KIINASE OXIDANT STRESS | D63780 | 424-711 |
| KINASE (YSK1, STE20 and SPS1 | | |
| RELATED KINASE) | | |
| SERUM | M63012 | 843-1099 |
| PARAOXONASE/ARYLESTERASE 1 (EC | | |
| 3.1.1.2) (EC 3.1.8.1) (PON 1) (SERUM | | |
| ARYLDIAKYLPHOSPHATASE 1) (A- | | |
| ESTERASE 1) (AROMATIC ESTERASE 1) | | |
| PONI OR PON. | | |
| CATECHOL 0-METHYLTRANSFERASE, | M65212 | 481-742 |
| MEMBRANE-BOUND FORM (EC 2.1.1.6) | | |
| (MB-COMT) (CONTAINS: CATECHOL 0- | | |
| METHYLTRANSFERASE, SOLUBLE | | |
| FORM (S-COMT)) COMT. | | |
| AMINE OXIDASE (FLAYIN- | M68840 | 268-516 |
| CONTAINING) A (EC 1.4.3.4) | | |
| (MONOAMINE OXIDASE) (MAO-A) | | |
| MAOA. | | |
| AMINE OXIDASE (FLAVIN- | M69177 | 130-358 |
| CONTAINING) B (EC 1.4.3.4) | | |
| (MONOAMINE OXIDASE) (MAO-B) | | |
| MAOB. | | |
| EUKARYOTIC PEPTIDE CHAIN | M75715 | 441-673 |
| RELEASE FACTOR SUBUNIT 1 (ERF1) | | |
| (TB3-1) (Cli PROTEIN) RFI. | | |
| UDP-GLUCURONOSYLTRANSFERASE | M84127 | 110-380 |
| 1-3 PRECURSOR, MICROSOMAL (BC | | |
| 2.4.1.17) (UDPGT) (UGT-1C) (UGT1*3) | | |
| (UGT 1-03) (UGT 1.3) (UGT 1A3) (UGT1 C) | | |
| UGTI ORGNT1. | | |
| STRUCTURE-SPECIFIC RECOGNITION | M86737 | 695-933 |
| PROTEIN 1 (SSRPI) (RECOMBINATION | | |
| SIGNAL SEQUENCE RECOGNITION | | |
| PROTEIN) (T160) SSRPI. | | |
| IJDP-GLUCURONOSYLTRANSFERASE | S55985 | 546-773 |
| 1-2 PRECURSOR, MICROSOMAL (BC | | |
| 2.4.1.17) (UDPGT) (UGT-1B) (UGT1*2) | | |
| (UGT 1-02) (UGT1 .2) (UGTI A2) (UGT iB) | | |
| (HLUGP4) UGT 1 OR GNT 1. | | |
| TH10PURJNE 5-METHYLTRANSFERASE | S62904 | 154-423 |
| (EC 2.1.1.67) (TH10PURTh4E | | |
| METI{YLTRANSFERASE) TPMT. | | |
| MELOTIC RECOMBINATION PROTEIN | D63882 | 573-898 |
| DMC1/LIM15 HOMOLOG | | |
| ACYL-COA DEHYDROGENASE, | U12778 | 668-904 |
| SHORT/BRANCHED CHAIN SPECIFIC | | |
| PRECURSOR (EC 1.3.99.-) (SBCAD) (2- | | |
| METHYL BRANCHED CHAIN ACYL- | | |
| COA DEHYDROGENASE) (2-MEBCAD) | | |
| ACADSB. | | |
| ARYLAMINE N-ACETYLTRANSFERASE, | X14672; X17059 | 800-1082 |
| POLYMORPHIC (EC 2.3.1.5) (PNAT) | | |
| ARYLAMINE N-ACETYLTRANSFERASE, | | |
| MONOMORPHIC (EC 2.3.1.5) (MNAT) | | |
| GLUTATH10NE PEROXIDASE- | X53463 | 369-595 |
| GASTROINTESTINAL (EC 1.11.1.9) | | |
| (GSHPX-GI) (GLUTATH10NE | | |
| PEROXIDASE-RELATED PROTEIN 2) | | |
| (GPRP) GPX2. | | |
| CYTOCHROME P450 XIB1 PRECURSOR | X55764 | 351-614 |
| (P450C1 1) (STEROID 11-BETA- | | |
| HYDROXYLASE) (EC 1.14.15.4) CYPI IBi | | |
| OR SI 1BH. | | |
| CYTOCHROME P450 IVAI 1 (EC 1.14.14.1) | X71480 | 148-385 |
| (FRAGMENT) CYP4A-1 1. | | |
| BLEOMYCIN HYDROLASE (EC 3.4.22.-) | X92106 | 1171-1408 |
| (BLM HYDROLASE). | | |
| NADH-CYTOCHROME B5 REDUCTASE | Y09501 | 307-556 |
| (EC 1.6.2.2) (B5R) DIAl. | | |
| COPROPORPHYRINOGEN III OXIDASE | Z28409 | 602-982 |
| PRECURSOR (EC 1.3.3.3) | | |
| (COPROPORPHYRINOGENASE) | | |
| (COPROGEN OXIDASE) (COX) CPO. | | |
| HEAT-SHOCK PROTEIN 110 KD | D86956 | 903-1153 |
| (KIAAO20 1) | | |
| GAMMA CRYSTALLIN C (GAMMA | U66582; M11971; | 5-249 |
| [M11970] | | |
| CRYSTALLIN 2 OR ⅓) (CRYGC) OR | | |
| (CRYG3). GAMMA CRYSTALLIN B | | |
| (GAMMA CRYSTALLIN 1-2) (CRYGB) | | |
| OR (CRYG2) (HUMCRYGX1). | | |
| HEAT SHOCK TRANSCRIPTION | D87673 | 542-794 |
| FACTOR 4. | | |
| CYTOCHROMEP450IVB1 (EC 1.14.14.1) | J02871 | 1015-1273 |
| (P450-HP) | | |
| EXTRACELLULAR SUPEROXIDE | J02947 | 870-1150 |
| DISMUTASE PRECURSOR (CU-ZN) (EC | | |
| 1.15.1.1) (EC-SOD) 50D3. | | |
| DNAJ PROTEIN HOMOLOG 2 (DNAJ2 | D13388 | 798-1095 |
| OR HDJ2) | | |
| DNA MISMATCH REPAIR PROTEIN | J04810 | 2807-3170 |
| MSH3 (DIVERGENT UPSTREAM | | |
| PROTEIN) (MISMATCH REPAIR | | |
| PROTEIN 1) (MRP1) (DUP) (DUG) | | |
| PROTEIN DISULFIDE ISOMERASE- | J05016 | 1009-1263 |
| RELATED PROTEIN PRECURSOR | | |
| (ERP72) | | |
| REPLICATION PROTEIN A 32 KD | J05249 | 195-420 |
| SUBUNIT (RP-A) (RE-A) (REPLICATION | | |
| FACTOR-A PROTEIN 2) | | |
| MU-CRYSTALLIN HOMOLOG (CRYM) | L02950 | 420-714 |
| (HUMMUCRYS). | | |
| MULTIDRUG RESISTANCE- | L05628 | 1891-2137 |
| ASSOCIATED PROTEIN I | | |
| REPLICATION PROTEIN A 14 KD | L07493 | 30-256 |
| SUBUNIT (RP-A) (RE-A) (REPLICATION | | |
| FACTOR A PROTEIN 3) | | |
| CALNEXIN PRECURSOR (MAJOR | L10284; [L18887; M94859; | 1146-1380 |
| HISTOCOMYATIBILITY COMPLEX | M98452] | |
| CLASS 1 ANTIGEN-BINDING PROTEIN | | |
| P88) (P90) (1P90) | | |
| CYCLOPHILIN.A0 | L11667 | 431-720 |
| HEAT SHOCK 70 KD PROTEIN 4 | L12723 | 1606-1994 |
| (HSP7ORY). | | |
| QUINONE OXIDOREDUCTASE (EC | L13278; [S58039] | 11-261 |
| 1.6.5.5) (NADPH:QUINONE REDUCTASE) | | |
| (ZETA-CRYSTALLIN). | | |
| T-COMPLEX PROTEIN 1, THETA | D13627 | 1025-1264 |
| SUBUNIT (TCP- 1 -THETA)(CCT-THETA) | | |
| (HUMRSC548). | | |
| REGULATED PROTEIN) (GRE 75) | L15189 | 289-543 |
| (PEPTIDE-BINDING PROTEIN 74) | | |
| (PBP74) (MORTALIN) (MOT). | | |
| P23 PROGESTERONE RECEPTOR | L24804; [L24805] | 240-649 |
| ASSOCIATED PROTEIN (HUMPRA) | | |
| FLAP ENDONUCLEASE.1 | L37374 | 559-794 |
| (MATURATION FACTOR 1) (MF1) (FEN- | | |
| 1) | | |
| DNA NUCLEOTIDYLEXOTRANSFERASE | M11722 | 1555-1843 |
| (TERMINAL ADDITION ENZYME) | | |
| (TERMINAL | | |
| DEOXYNUCLEOTIDYLTRANSFERASE) | | |
| (TERMINAL TRANSFERASE) (DNTT) | | |
| (TDT) | | |
| POLY (ADP-RIBOSE) POLYMERASE | M18112 | 2663-2900 |
| (PARE) (ADPRT) (NAD (+) ADP- | | |
| RIBOSYLTRANSFERASE) (POLY (ADP- | | |
| RIBOSE) SYNTHETASE) (PPOL) | | |
| V(D)J RECOMBINATION ACTIVATING | M29474 | 841-1145 |
| PROTEIN 1 (RAG1) (RAG-1) | | |
| FK506-BINDING PROTEIN (FKBP) | M34539; [M80199; | 302-565 |
| (FKBP 12) (PEPTIDYL-PROLYL CIS- | M80706; M92423; J05340; | |
| TRANS ISOMERASE) (PPIASE) | X55741; X52220] | |
| (ROTAMASE) | | |
| DNA LIGASE I | M36067 | 1739-2049 |
| (POLYDEOXYRIBONUCLEOTIDE | | |
| SYNTHASE (ATP)) (DNL1) (LIG1) | | |
| RAPAMYC1N-BINDTNG PROTEIN (FKBP- | M65128 | 135-370 |
| 13) | | |
| DNA-REPAIR PROTEIN | D14533 | 1086-1316 |
| COMPLEMENTING XP-A CELLS | | |
| (XERODERMA PIGMENTOSUM GROUP | | |
| A COMPLEMENTING PROTEIN) | | |
| HEAT SHOCK FACTOR PROTEIN 2 (11SF | M65217 | 1437-1671 |
| 2) (HEAT SHOCK TRANSCRIPTION | | |
| FACTOR 2)(HSTF 2). | | |
| DNA-3 METHYLADENINE | M74905 | 459-699 |
| GLYCOSYLASE (3-METHYLADENINE | | |
| DNA GLYCOSYLASE) (ADPG) (3- | | |
| ALKYLADENINE DNA GLYCOSYLASE) | | |
| (N-METHYLPURINE-DNA | | |
| GLYCOSIRASE) (MPG) (MAGI) (3MeAG) | | |
| DNA POLYMERASE DELTA CATALYTIC | M80397 | 1629-1913 |
| CHAIN | | |
| CALRETICIJLIN PRECURSOR (CRP55) | M84739 | 214-501 |
| (CALREGULIN) (HACBP) (ERP6O)(52 KD | | |
| RIBONUCLEOPROTEIN AUTOANTIGEN | | |
| RO/SS-A) | | |
| TRANSFORMATION -SENSITIVE | M86752 | 1333-1646 |
| PROTEIN (LEF SSP 3521) | | |
| ALPHA CRYSTALLIN B CHAIN | S45630 | 368-669 |
| (ALPHA(B)-CRYSTAILIN) (ROSENTHAL | | |
| FIBER COMPONENT). | | |
| HEAT SHOCK PROTEIN H5P72 | S67070 | 69-378 |
| HOMOLOG (FRAGMENT). | | |
| ALPHA CRYSTALLIN A CHAIN | U05569 | 54-374 |
| (H5U05569). | | |
| NICOTINAMIDE N- | U08021 | 124-385 |
| METHYLTRANSFERASE (EC 2.1.1.1) | | |
| IJDP-GLUCURONOSYLTRANSFERASE | U08854; X63359; U06641; | 1268-1509 |
| 2B15 PRECURSOR, MICROSOMAL (EC | 105428; Y00317 | |
| 2.4.1.17) (UDPGT) (UDPGTH-3) UGT2B15. | | |
| UDP-GLUCURONOSYLTRANSFERASE | | |
| 2B10 PRECURSOR, MICROSOMAL (EC | | |
| 2.4.1.17) (UDPGT) UGT2B 10. UDP- | | |
| GLUCURONOSYLTRANSFERASE 2B8 | | |
| PRECURSOR, MICROSOMAL (EC | | |
| 2.4.1.17) | | |
| PROBABLE PROTEIN DISULFIDE | D16234; [Z49835; D83485; | 788-1019 |
| ISOMERASE ER-60 PRECURSOR (EC | U42068] | |
| 5.3.4.1) (ERP6O) (58KDA MICROSOMAL | | |
| PROTEIN) (phospholipase C-alpha) | | |
| PHENOL-SULFATING PHENOL | U09031; U28170; L19956 | 202-465 |
| SULFOTRANSFERASE 1 (EC 2.8.2.1) (P- | | |
| PST) (THERMOSTABLE PHENOL | | |
| SULFOTRANSFERASE) (TS-PST) | | |
| (HAST1/HAST2) (ST1A3) STP1 OR STP. | | |
| PHENOL-SULFATING PHENOL | | |
| SULFOTRANSFERASE 2 (EC 2.8.2.1) (P- | | |
| PST) (ST1A2) STP2. MONOAMINE- | | |
| SULFATING PHENOL SU | | |
| DIHYDROPYRIMIDINE | U09178 | 2257-2590 |
| DEHYDROGENASE (NADP+) | | |
| PRECURSOR (EC 1.3.1.2) (DPD) | | |
| (DIHYDROURACIL DEHYDROGENASE) | | |
| (DIHYDROTHYMINE | | |
| DEHYDROGENASE) DPYD. | | |
| X-LINKED HELICASE II (X-LINKED | U09820 | 4521-4849 |
| NUCLEAR PROTEIN) (XNP) (RAD54L) | | |
| (XH2) | | |
| TUMOR NECROSIS FACTOR TYPE 1 | U12595 | 716-1013 |
| RECEPTOR ASSOCIATED | | |
| PROTEIN(TRAP l)(HSU 12595) | | |
| TUMOR NECROSIS FACTOR TYPE 1 | U12596 | 1882-2202 |
| RECEPTOR ASSOCIATED | | |
| PROTEIN(TRAP2) (HSUI 2596) | | |
| DAMAGE-SPECIFIC DNA BINDING | U18299 | 371-645 |
| PROTEIN piT? SUBUNIT; IMPLICATED | | |
| iN XERODERMA PIGMENTOSUM | | |
| GROUP E (DDBI) | | |
| DAMAGE-SPECIFIC DNA BINDING | U18300 | 1042-1316 |
| PROTEIN p48 SUBUNIT; IMPLICATED IN | | |
| XERODERMA PIGMENTOSUM GROUP E | | |
| (DDB2) | | |
| COCKAYNE SYNDROME GROUP A; WD- | U28413 | 296-599 |
| REPEAT PROTEIN (CSA PROTEIN) | | |
| HSC70-1NTERACTING PROTEIN | U28918 | 493-737 |
| (PROGESTERONE RECEPTOR- | | |
| ASSOCIATED P48 PROTEIN) | | |
| T-COMPLEX PROTEIN 1, DELTA | U38846 | 1063-1356 |
| SUBUNIT (TCP- I-DELTA)(CCT-DELTA) | | |
| (STIMULATOR OF TAR RNA BINDING) | | |
| (H5U38846). | | |
| 7,8-DIHYDRO-8-OXOGUANINE | D16581 | 221-455 |
| TRIPHOSPHATASE (mutT | | |
| HOMOMOLOG) (8-OXO-DGTPASE) | | |
| (MTH1) | | |
| DNA REPAIR PROTEIN XRCC4 | U40622 | 718-969 |
| DNA TOPOISOMERASE III (TOP3) U43431 | 1534-1785 | |
| G/T MISMATCH-SPECIFIC THYMINE | U51166 | 830-1093 |
| DNA GLYCOSYLASE (TDG) | | |
| 150 KDA OXYGEN-REGULATED | U65785 | 1471-1761 |
| PROTEIN ORP 150 (HSU65785) | | |
| DNA REPAIR PROTEIN XRCC9 | U70310 | 1715-2008 |
| 48 kDa FKBP-ASSOCIATED PROTEIN | U73704 | 954-1228 |
| FAP48 | | |
| ENDONUCLEASE III HOMOLOG 1 | U79718 | 192-455 |
| (HNTH1) (OCTS3) | | |
| T-COMPLEX PROTEIN 1, ETA SUBUNIT | U83843 | 885-1144 |
| (TCP-1-ETA) (CCT-ETA)(HIV-1 NEF | | |
| INTERACTING PROTEIN) (HSU83843). | | |
| CATALASE (EC 1.11.1.6) CAT. | X04076 | 957-1214 |
| PORPHOBILINOGEN DEAMINASE (EC | X04808 | 736-998 |
| 4.3.1.8) (HYDROXYMETHYLBILANE | | |
| SYNTHASE) (HMBS) (PRE- | | |
| UROPORPHYRINOGEN SYNTHASE) | | |
| DNA-REPAIR PROTEIN | D21089 | 1767-2092 |
| COMPLEMENTING XP-C CELLS | | |
| (XERODERMA PIGMENTOSUM GROUP | | |
| C COMPLEMENTING PROTEIN) (p 125) | | |
| HEME OXYGENASE 1 (EC 1.14.99.3) (HO- | X06985 | 791-1056 |
| 1) (HSOXYGR). | | |
| SUPEROXIDE DISMUTASE PRECURSOR | X07834; [X59445] | 89-348 |
| (MN) (EC 1.15.1.1) 50D2 | | |
| ENDOPLASMIN PRECURSOR (94 KD | X15187; [M33716] | 1357-1645 |
| GLUCOSE-REGULATED | | |
| PROTEIN)(GRP94) (GP96 HOMOLOG) | | |
| (TUMOR REJECTION ANTIGEN 1) | | |
| (LISTRAI). | | |
| URACIL-DNA GLYCOSYLASE | X15653 | 547-772 |
| PRECURSOR (UNGl) | | |
| NCK MELANOMA CYTOPLASMIC SRC | X17576 | 404-675 |
| HOMOLOGUE (HSNCK) | | |
| 5,6-DIHYDROXYINDOLE-2- | X51420 | 909-1145 |
| CARBOXYLIC ACID OXIDASE | | |
| PRECURSOR (DHICA OXIDASE) | | |
| (TYROSINASE-RELATED PROTEIN 1) | | |
| (TRP-1) (CATALASE B) | | |
| (GLYCOPROTEIN-75) (GP75) | | |
| URACIL-DNA GLYCOSYLASE 2 (UNG2) | X52486 | 882-1118 |
| T-COMPLEX PROTEIN 1, ALPHA | X52882 | 993-1336 |
| SUBUNIT (TCP-1-ALPHA)(CCT-ALPHA) | | |
| CCT1 OR CCTA OR TCP1 | | |
| 40S RIBOSOMAL PROTEIN S3 | X55715 | 315-621 |
| (POSSIBLE dRpase) | | |
| DNA-(APURINIC OR APYRIMIDINIC | X59764; [X66133] | 524-768 |
| SITE) LYASE (AP ENDONUCLEASE 1) | | |
| (APEX NUCLEASE) (APEN) (REF-1 | | |
| PROTEIN) (APEl) | | |
| HEME OXYGENASE 2 (EC 1.14.99.3) (HO- | D21243; [S34389] | 508-763 |
| 2) | | |
| 47 KD HEAT SHOCK PROTEIN | X61598; D83174 | 539-777 |
| PRECURSOR (COLLAGEN-BINDING | | |
| PROTEIN 1) (COLLIGIN 1) + Collagen | | |
| binding protein 2 (HUMCBP2). | | |
| T-COMPLEX PROTEIN 1, GAMMA | X74801; [U17104] | 32-270 |
| SUBUNIT (TCP- 1 -GAMMA)(CCT- | | |
| GAMMA) (CCT3) OR (CCTG) OR (TRICS) | | |
| (I- SHUMAPC). | | |
| DNA REPAIR PROTEIN RAD54 | X97795 | 1001-1284 |
| HOMOLOG | | |
| BASIC TRANSCRIPTION FACTOR 2,52 | Y07595 | 466-830 |
| KD SUBUNIT (BTF2pS2) | | |
| RAD5 1-LIKE PROTEIN (POSSIBLE | Y08837 | 203-266 |
| XRCC2) | | |
| 8-OXYGUANINE DNA GLYCOSYLASE | Y11838 | 380-646 |
| HOMOLOG 1 (mutM HOMOLOG) (OGH1) | | |
| (HOGGI) (FaPyG) | | |
| CYTOCHROME P450 1A2 (EC 1.14.14.1) | Z00036 | 1221-1509 |
| (P450-P3) (P450-4).5 15 | | |
| BASIC TRANSCRIPTION FACTOR 2,34 | Z30093 | 585-899 |
| KD SUBUNIT (BTF2p34) | | |
| DIMETHYLANILINE | L37080 | 1086-1376 |
| MONOOXYGENASE (N-OXIDE | | |
| FORMING) 5 (EC 1.14.13.8) (HEPATIC | | |
| FLAVIN-CONTAINING | | |
| MONOOXYGENASE 5) (FMO 5) | | |
| (DIMETHYLANILINE OXIDASE 5) | | |
| UBIQUITJN-LIKE PROTEIN (NEDD8) | D23662 | 222-546 |
| MULTIDRUG RESISTANCE PROTEIN 3 | M23234 | 1798-2094 |
| (P-GLYCOPROTE1N 3) | | |
| DIMETHYLANILINE | M64082 | 721-966 |
| MONOOXYGENASE (N-OXIDE | | |
| FORMING) 1 (EC 1.14.13.8) (FETAL | | |
| HEPATIC FLAVIN-CONTAINING | | |
| MONOOXYGENASE 1) (FMO 1) | | |
| (DIMETHYLANILINE OXIDASE 1) | | |
| HHR6B (YEAST RAD6 HOMOLOG) | M74525 | 523-761 |
| (UBIQITIN-CON.TUGATING ENZYME) | | |
| (UBCB) | | |
| IMMUNOPHILLIN (FKBP52) | M88279 | 554-837 |
| HEAT SHOCK PROTEIN HSP40HEAT | U40992 | 13-300 |
| SHOCK PROTEIN HSP40 HOMOLOG. | | |
| 54 KDA PROGESTERONE RECEPTOR- | U42031 | 829-1147 |
| ASSOCIATED PROTEIN FKBP54 | | |
| HEMATOPOJETIC PROGENITOR | U66464 | 264-536 |
| KINASE ACTIVATOR OF SAPK/JNK | | |
| (HPK1) (H5U66464) | | |
| SPS1/STE20 HOMOLOGUE, KHS, | U77129 | 1376-1633 |
| ACTIVATOR OFJUN N-TERMINAL | | |
| KINASE (H5U77 129) | | |

### EXAMPLE 2

### Alternative Human Stress Array

An alternative array to that described in Example 1 displays probes obtained from the list of genes appearing in Table 2.

TABLE 2

| Clon ID | Gene Name | Gen Bank Acc # | Sprot Acc # |
|---|---|---|---|
| | interleukin 7 receptor alpha subunit (IL7R-alpha; | | |
| 1 | IL7RA); CDW127 antigen | M29696 | P16871 |
| 4 | myc proto-oncogene | V00568 | P01106; P01107 |
| | interleukin 2 receptor alpha subunit (IL2 receptor | X01057; X01058; | |
| 5 | alpha; IL2RA); TAG antigen; CD25 antigen | X01402 | P01589 |
| 9 | insulin-like growth factor II (IGF2); somatomedin A | M29645 | P01344 |
| | tumor necrosis factor receptor superfamily member | | |
| | 1B (TNFRSF1B); tumor necrosis factor receptor 2 | | |
| | (TNFR2); tumor necrosis factor beta receptor | | |
| 13 | (TNFBR); CD120B antigen | M32315; M55994 | P20333 |
| | p53-binding mouse double minute 2 homolog | | |
| 14 | (MDM2) | Z12020; M92424 | Q00987; Q13226 |
| 19 | interleukin 1 beta (ILl-beta; ILIB); catabolin | K02770 | P01584 |
| 34 | transforming growth factor beta (TGF-beta; TGFB) | X02812; J05114 P01137 | |
| 37 | mybproto-oncogene | M15024 | P10242 |
| 38 | p53 cellular tumor antigen | M14694; M14695 | P04637 |
| | transforming growth factor beta 2 (TGF-beta 2; | M19154; M22045; | |
| 39 | TGFB2) | M22046;Y00083 | P08112; Q15581 |
| | interleukin 4 receptor alpha subunit (IL4R-alpha; | | |
| 46 | IL4RA); CD124 antigen | X52425 | P24394 |
| | interferon aiphalbeta/omega receptor subunit 2 | | |
| 50 | (IFN-alpha receptor 2; IFNAR2) | X77722 | P48551 |
| | interferon gamma-induced monokine (MIG); small | | |
| 51 | inducible cytokine subfamily B member 9 (SCYB9) X72755 | Q07325 | |
| | interleukin 2 receptor gamma subunit (IL2R- | | |
| | gamma; IL2RG); cytokine receptor common gamma | | |
| 54 | chain | D11086 | P31785 |
| | interferon gamma receptor 1 (IFNR-gamma 1; | | |
| 58 | IFNGR1); immune interferon receptor 1 | J03143 | P15260 |
| 61 | adenosine Al receptor (ADORA1) | 556143 | P30542 |
| 62 | orphan hormone nuclear receptor | Z30425 | Q14994 |
| 63 | adenosine A3 receptor (ADORA3) | X76981 | P33765 |
| 66 | thrombin receptor (TR); F2R; PAR1 | M62424 | P25116 |
| | transforming growth factor beta receptor III (TGF | | |
| 67 | beta receptor III; TGFR3); betaglycan | L07594 | Q03167 |
| 72 | GATA-binding protein 2 (GATA2) | M68891 | P23769 |
| | CCAAT-binding transcription factor subunit B | | |
| | (CBFB); CBFA; NF-Y protein subunit A (NF-YA); | | |
| 76 | HAP2 | M59079 | P23511 |
| | retinoic acid receptor beta (RAR-beta; RARB); | | |
| 77 | hepatitis B virus-activated protein (HAP) | X07282; Y00291 | P10826 |
| | MYB-related protein B (B-MYB); avian | | |
| | myeloblastosis viral oncogene homolog-like 2 | | |
| 78 | (MYBL2) | X13293 | P10244 |
| 79 | tyrosine kinase receptor tie-I | X60957; 589716 | P35590 |
| 81 | inhibitor of DNA-binding protein 3 (103); HEIR1 | X69111 | Q02535; 075641 |
| | basic transcription element-binding protein 2 | | |
| 83 | (BTEB2); GC-box binding protein 2 | 014520 | Q13887 |
| 84 | basic transcription factor 62-kDa subunit (BTF2) | M95809 | P32780 |
| 88 | DNA-binding protein SMBP-2; glial factor-1 (GF-1) | L14754 | P38935 |
| | SWI/SN F related matrix-associated actin- | | |
| | dependent regulator of chromatin subfamily a | | |
| | member 1 (SMARCA1); sucrose nonfermenting 2 | | |
| 90 | homolog-like protein 1 (SN F2L1) | M881 63 | P28370 |
| | interferon consensus sequence-binding protein | | |
| 96 | (ICSBP) | M91196 | Q02556 |
| 98 | CACCC-box DNA-binding protein | L04282 | Q15552 |
| 104 | activating transcription factor 3 (ATF3) | L19871 | P18847 |
| | neurotrophic tyrosine kinase receptor-related 3; | | |
| 107 | TKT | X74764 | Q16832 |
| 109 | prostaglandin E2 receptor EP4 subtype (PTGER4) | L25124; D28472 | P35408 |
| | thyroid hormone receptor alpha 2 (THRA2); erbA | | |
| 119 | alpha2proto-oncogene | J03239 | P10827 |
| 123 | amino-terminal enhancer of split (AES) | X73358 | Q08117; Q12808 |
| | DNA-binding protein HIPI16; ATPase; SNF2/SW12- | | |
| 124 | related protein | L34673 | Q14527 |
| | zinc finger protein 9 (ZFN9); cellular nucleic acid- | | |
| 127 | binding protein (CNBP) | M28372 | P20694 |
| | v-erbA related protein 3 (EAR3); transcription factor | | |
| | COUP2 (TFCOUP2); nuclear receptor subfamily 2 | Xl 2795; Xl 6155; | |
| 129 | group F member 2 (NR2F2) | X58241 | P10589 |
| | cut-like protein 1 (CUTL1); CCAAT displacement | | |
| 130 | protein (CDP) | L12579 | Q13948 |
| | basic transcription factor 244-kDa subunit | | |
| 132 | (BTF2p44) | Z30094 | Q13888 |
| | signal transducer and activator of transcription 3 | | |
| 134 | (STAT3); acute-phase response factor (APRF) | L29277 | P40763 |
| | SWI/SN F-related matrix-associated actin- | | |
| | dependent regulator of chromatin subfamily A | | |
| | member 4 (SMARCA4); sucrose nonfermenting 2 | | |
| | homolog-like 4 (SNF2L4); brahma-related gene 1 | | |
| 136 | (BRGI) | D26156 | P51532 |
| 142 | transcriptional repressor NF-X1 | U15306 | Q12986 |
| | transcription factor DP2 (Humdp2); E2F | | |
| 143 | dimerization partner 2 | U18422 | Q14188; Q14187 |
| 144 | glia maturation factor beta (GMF-beta; GMFB) | M86492 | P17774 |
| | cAMP-responsive element-binding protein 1 | | |
| 147 | (CREB1) | L05515 | Q02930; Q05886 |
| | protein-tyrosine phosphatase gamma (R-PTP- | | |
| 149 | gamma) | L09247 | P23470 |
| | activator 1140-kDa subunit (A1 140-kDa subunit); | | |
| | replication factor C large subunit; DNA-binding | | |
| 151 | protein PO-GA | L14922 | P35251 |
| | heat shock transcription factor 1 (HSTF1; HSF1); | | |
| 152 | transcription factor 5 (TCF5) | M64673 | Q00613 |
| 153 | c-jun proto-oncogene | J04111 | P05412 |
| | interleukin 1 receptor type 1 (IL1R1); IL1R-alpha; | | |
| 154 | p80;CDW121Aantigen | M27492 | P14778 |
| | macrophage-specific colony-stimulating factor | | |
| 156 | (CSF-1; MOSE) | M37435 | P09603 |
| | insulin-like growth factor 2 receptor (IGF2R); cation- | | |
| | independent mannose-6-phosphate receptor | | |
| 158 | (MPIR) | Y00285; J03528 | P11717 |
| | CXC chemokine receptor type 4 (CXCR4); stromal | | |
| | cell-derived factor 1 receptor (SDF1 receptor); | | |
| | fusin; leukocyte-derived seven transmembrane | | |
| 164 | domain receptor (LESTR); LCR1 | D10924 | P30991; P56438 |
| | CC chemokine receptor type 1 (CMKBRI; | | |
| | CCCKR1; CCR1); macrophage inflammatory | | |
| | protein 1 alpha receptor (MIP1-alpha receptor; | | |
| 165 | MIPI-alphaR) | D10925 | P32246 |
| | macrophage inflammatory protein 1 beta (MIP1- | | |
| | beta); T-cell activation protein 2 (AT2); PAT 744; | | |
| | H400; SIS-gamma; lymphocyte activation gene 1 | | |
| | protein (LAG 1); HC21; small inducible cytokine | | |
| | subfamily A member 4 (SCYA4); G 26 T- | | P13236; P22617; |
| 171 | lymphocyte secreted protein | J04130 | Q13704 |
| | glucose-6-phosphate isomerase (GPI); neuroleukin | | |
| | (NLK); phosphoglucose isomerase (PGI); | | |
| 174 | phosphohexose isomerase (PHI) | K03515 | P06744 |
| | angiopoietin 1 receptor; tyrosine-protein kinase | | |
| | receptor TIE-2; tyrosine-protein kinase receptor | | |
| | TEK; p140 TEK; tunica interna endothelial cell | | |
| 175 | kinase | L06139 | Q02763 |
| 178 | interleukin 13 (IL13) | L06801 | P35225 |
| 187 | thrombopoietin (THPO); megakaryocyte colony | L36052; L36051; | P40225 |
| | stimulating factor; c-mpl ligand; megakaryocyte | U11025 | |
| | growth & development factor (MGDF) | | |
| | uromodulin; Tamm-Horsfall urinary glycoprotein | | |
| 193 | (THP) | M17778 | P07911 |
| | platelet-derived growth factor receptor alpha | | |
| 196 | (PDGFR-alpha; PDGFRA); CD14QA antigen | M21574 | P16234 |
| | platelet-derived growth factor receptor beta subunit | | |
| 197 | (PDGFR-beta; PDGFRB); CD140B antigen | M21616 | P09619 |
| | bone morphogenetic protein 1 (BMPI); procollagen | | |
| 198 | C proteinase 2 (PCP2) | M22488; U50330 | P13497; Q13292 |
| 199 | bone morphogenetic protein 2A (BMP2A) | M22489 | P12643 |
| | macrophage inflammatory protein 1 alpha (MIP1- | | |
| | alpha); tonsillar lymphocyte LD78 alpha protein; | | |
| | GOS19-1 protein; PAT 464.2; SIS-beta; small | | |
| 201 | inducible cytokine subfamily A member 3 (SCYA3) | M23452 | P10147; P16619 |
| | small inducible cytokine subfamily A member 2 | | |
| | (SCYA2); monocyte chemotactic protein 1 (MCP1); | | |
| 202 | monocyte chemotactic & activating factor (MOAF) | M24545 | P13500 |
| | insulin-like growth factor binding protein 1 (IGF- | | |
| | binding protein 1; IGFBPI); placental protein 12 | | |
| 208 | (PP12) | M31145 | P08833 |
| | tumor necrosis factor alpha-induced protein 6 | | |
| 209 | (TNFAIP6); TSG6 | M31165 | P98066 |
| | vascular endothelial growth factor (VEGF); vascular | | |
| 210 | permeability factor (VPF) | M32977; M27281 | P15692 |
| | insulin-like growth factor-binding protein 2 (IGF- | | |
| 211 | binding protein 2; IGFBP2; IBP2) | M35410 | P18065; Q14619 |
| | ribon uclease/angiogenin inhibitor (RAI; RN H); | | |
| 212 | placental ribonuclease inhibitor | M36717 | P13489 |
| | fibroblast growth factor receptori (FGFRI); basic | X66945; M34641; | |
| | fibroblast growth factor receptor (BFGFR); fins-like M34186; M37722; | | |
| | tyrosine kinase 2 (FLT2); heparin-binding growth | M83887; M63888; | P11362; P17049; |
| 214 | factor receptor alpha A (HBGFR-alpha A) | M63889 | Q02063; Q02065 |
| | plelotrophin (PTN); osteoblast specific factor 1 | | |
| | (OSF1); heparin-binding neurite growth-promoting | | |
| | factor 1 (HBNF1; NEGF1); heparin-binding growth- | | |
| | associated molecule (HB-GAM); heparin-binding | X52946; D90226; | |
| 216 | growth factor 8 (HBGF8) | M57399 | P21246 |
| | interleukin 11 (ILl1); adipogenesis inhibitory factor | | |
| 218 | (AGIF) | M57765 | P20809 |
| | stem cell factor (SCF); mast cell growth factor | | |
| 222 | (MGF); c-kit ligand | M59964 | P21583 |
| | tumor necrosis factor receptor superfamily member | | |
| 235 | 7 (TNFRSF7); CD27L antigen receptor | M63928 | P26842 |
| 247 | thymosin beta 10 (TMSB10; THYB10); PTMB10 | M92381 | P13472 |
| 248 | connective tissue growth factor (CTGF) | M92934 | P29279 |
| 252 | related to receptor tyrosine kinase (RYK) | S59184 | P34925 |
| | Duffy blood group antigen; FY glycoprotein (GPFY); | | |
| 255 | glycoprotein D (GPD); DARC | U01839 | Q16570; Q16300 |
| | SL cytokine; FMS-related tyrosine kinase 3 ligand | | |
| 263 | (FLT3 ligand; FLT3LG) | U04806; U03858 | P49771 |
| | caspase 3 (CASP3); apopain; cysteine protease | | |
| | GPP32; YAMA protein; SREBP deavage activity 1 | | |
| 266 | protein (SCA1) | U13737 | P42574 |
| | calgranulin A (GALA); migration inhibitory factor- | | |
| | related protein 8 (MRP8); leukocyte LI complex | | |
| | light chain; SiQO calcium-binding protein A8 | | |
| 276 | (S100A8); cystic fibrosis antigen (OFAG) | X06234 | P05109 |
| | platelet-derived growth factor A subunit (PDGFA; | | |
| 277 | PDGF1) | X06374 | P04085 |
| | fins-related tyrosine kinase 1 (ELT1); vascular | | |
| | endothelial growth factor receptor 1 (VEGERI); | | P17948; P16057; |
| 285 | vascular permeability factor receptor | X51602 | 060722 |
| | placental growth factor (PLGF; PGF); vascular | | |
| 290 | endothelial growth factor-related protein | X54936 | P49763 |
| | tumor necrosis factor receptor superfamily member | | |
| 294 | 5 (TN FRSFS); 0040 antigen | X60592 | P25942 |
| | corticotropin-releasing factor receptor 1 (CRFR; | | |
| | GRE 1); corticotropin-releasing hormone receptor 1 | | |
| 296 | (CRHR1) | X72304 | P34998; Q13008 |
| | | | P07602; P07292; |
| | prosaposin (PSAP); variant Gaucher disease & | | P15793; P78538; |
| | variant metachromatic leukodystrophy protein; | | P78546; P78547; |
| 301 | sphingolipid activator protein 1 (SAP1); GLBA | D00422 | Q92739 |
| 302 | protein kinase G delta (PKC-delta) | D10495 | 015144 |
| | GA-binding protein alpha subunit (GABP-alpha; | | |
| | GABPA); transcription factor E4TF1-47; nuclear | | |
| 305 | respiratory factor 2 alpha subunit | D13316 | 006546; 012939 |
| | | | Q06545 |
| | GA-binding protein transcription factor alpha | | |
| 307 | subunit (GABPA); E4TFIA; E4TFI-60 | 013318 | Q06546; Q12939 |
| | DNA damage repair & recombination protein 51 | | |
| 308 | homolog (RAD51) | D13804 | 006609 |
| | alpha 1 catenin (GTNNA1); cadherin-associated | Q13866; Q14705; | |
| 309 | protein | L23805; L22080 | P35221 |
| | | | P41134; Q16377 |
| | | | Q16371; Q00651; |
| 310 | inhibitor of DNA binding 1 protein (101) | D13889 | Q00652 |
| | | | P46966; P46968; |
| 314 | defender against cell death 1 protein (DAD1) | Q15057 | Q08552; Q70364 |
| | TYRO3 tyrosine-protein kinase receptor; RSE; | | |
| 316 | SKY; DTK | Q17517 | Q06418 |
| | bone marrow stromal antigen 1 (BST-1); ADP- | | |
| | ribosyl cyclase 2; cyclic ADP-ribose hydrolase 2 | | |
| 317 | (GADPR hydrolase 2); GD157 antigen | Q21878 | Q10588 |
| 323 | LIM domain kinase 1 (LIMKI) | Q26309 | P53667 |
| | neuroblastoma suppression of tumorigenicity | | |
| | protein 1 (NBL1); differential screening-selected | | |
| 328 | gene aberrant in neuroblastoma (DAN) | D28124 | P41271 |
| | excision repair cross-complementing rodent repair | | |
| | deficiency complementation group 6 (ERCC6); | | |
| 334 | Cockayne syndrome protein 2 type B (CSB) | L04791 | Q03468 |
| | mitogen-activated protein kinase kinase 1 (MAP | | |
| | kinase kinase I; MAPKKI; MAP2K1; PRKMK1); | | |
| 335 | MAPK/ERK kinase 1 (MEK1) | L05624 | Q02750 |
| | replication factor C 36-kDa subunit (RFC36); | | |
| 341 | activator 1 36-kDa subunit | L07540 | P40937 |
| | replication factor C 38-kDa subunit (RFC38); | | |
| 342 | activator 1 38-kDa subunit | L07541 | P40938 |
| 344 | MAX-interacting protein 1 (MXI1) | L07648 | P50539; Q15887 |
| | sex-determining region Y protein (SRY); testis- | | |
| 347 | determining factor (TOF) | L10101 | Q05066 |
| | MOESIN-ezrin-radixin-like protein (MERLIN); L11353; Z22664; | | |
| 348 | schwannomin (SCH); neurofibromatosis 2 (NF2) | X72657; L27133 | P35240 |
| | focal adhesion kinase (FADK); proline-rich tyrosine | | |
| 350 | kinase 2 (PYK2) | LI 3616 | Q05397 |
| 351 | tyrosine-protein kinase ack | L13738 | Q07912 |
| 352 | early response protein NAKI; TR3 orphan receptor | L13740 | P22736 |
| | RAR-related orphan receptor alpha (ROR-alpha; | | |
| 353 | RORA); RZRA | U04897; L14611 | P35397; P45445 |
| | tight junction protein 1 (TJPI); zonula occludens | | |
| 357 | (ZOI) | L14837 | Q07157 |
| | nucleoside diphosphate kinase B (NDP kinase B; | | |
| | NDKB); expressed in non-metastatic cells 2 protein | | |
| | (NME2); myc purine-binding transcription factor | | |
| 359 | (PUF); NM23B | L16785; M36981 P22392 | |
| | relA proto-oncogene; nuclear factor of kappa light | | |
| 360 | polypeptide gene enhancer in B-cells 3 (NFKB3) | L19067 | Q04206 |
| | xeroderma pigmentosum group G complementing | | |
| | protein (XPG); X-ray repair-complementing | | |
| | defective repair in Chinese hamster cells 5 | | |
| 365 | (XRCC5) | L20046; X69978 | P28715 |
| | serine/threonine-protein kinase NRK2; | | |
| 367 | serine/threonine kinase 2 (STK2) | L20321 | P51957 |
| | ras-related associated with diabetes protein | | |
| | (RRAD); ras associated with diabetes protein 1 | | |
| 374 | (RAD1) | L24564 | P55042 |
| | calcium/calmodulin-dependent protein kinase type | | |
| 375 | IV catalytic subunit (CAMK IV); CAM kinase-GR | L24959 | Q16566 |
| | growth factor receptor-bound protein 2 (GRB2); | P29354; Q63057; | |
| 377 | abundant SRC homology protein (ASH) | L2951 1; M96995 | Q14450 |
| 378 | nuclear factor l-X (NFI-X) | L31881 | Q13050 |
| | mitogen-activated protein kinase kinase kinase 11 | | |
| | (MAPKKK11; MAP3K11); mixed lineage kinase 3 | | |
| 379 | (MLK3); SPRK; protein tyrosine kinase 1 (PTKI) | L32976 | Q16584 |
| | cyclin-dependent kinase 10 (CDKl 0); CDC2-related | | |
| 380 | protein kinase PISSLRE | L33264 | Q15130; Q15131 |
| 381 | RNA polymerase II elongation factor SIII p15 | L34587 | Q15369 |
| | subunit | | |
| | guanine nucleotide-binding protein alpha | | |
| 387 | stimulating activity polypeptide 1 (GNASI) | M14631 | P04895 |
| 391 | T-lymphocyte maturation-associated protein MAL | M15800 | P21145 |
| 393 | homeobox protein B7 (HOXB7); HOX2C; HHO.C1 | M16937 | P09629; Q15957 |
| | INT-2 proto-oncogene protein (fibroblast growth | | |
| 397 | factor-3) (FGF-3) (HBGF-3) | M17446 | P08620 |
| | B-lymphocyte CD19 antigen; B-lymphocyte surface | | |
| 398 | antigen B4; leu-12 | M21097 | P15391 |
| | protein kinase C alpha polypeptide (PKC-alpha; | | |
| 399 | PKCA) | M22199 | Q15137 |
| | prothymosin alpha (PTMA); thymosin alpha | | |
| 402 | (TMSA) | M26708 | Q15249; Q15592 |
| 406 | ras-related protein RAB2 | M28213 | P08886 |
| | stem cell protein (SCL); T-cell leukemia/lymphoma- | | |
| | 5 protein (TOLS); T-cell acute lymphocytic | | |
| 411 | leukemia-I protein (TAL1) | M29038 | P17542 |
| | Ku (p701p80) subunit; ATP-dependent DNA | | |
| | helicase II 86-kDa subunit: lupus ku autoantigen | | |
| | protein; thyroid-lupus autoantigen (TLAA); CTC box | | |
| | binding factor 85-kDa subunit (CTCBF; CTC85); | | |
| 417 | nuclear factor IV | M30938 | P13010 |
| | transcription factor E2-alpha (E2A); immunoglobulin | | |
| | enhancer binding factor E12; transcription factor-3 | | |
| 420 | (TCF3) | M31523 | P15923 |
| | activating transcription factor 2 (ATF2); cAMP | | |
| | response element DNA-binding protein 1 | | |
| 421 | (CREBP1); HB16 | M31630 | P15336 |
| | xeroderma pigmentosum group B complementing | | |
| | protein (XPB); excision repair cross-complementing | | |
| | rodent repair deficiency complementation group 3 | | |
| | (ERCC3); basal transcription factor 2 89-kDa | | |
| 422 | subunit (BTF2p89; TFIIH 89-kDa subunit) | M31899 | P19447 |
| | G22P1; Ku 70-kDa subunit (KU70); 70-kDa thyroid | | |
| 424 | autoantigen (TLAA) | M32865; S38729 | P12956 |
| | cadherin 2 (CDH2); neural cadherin (N-cadherin; | M34064; X57548; | |
| 426 | NOAD) | X54315; S42303 | P19022; 014923 |
| 427 | cAMP-response element binding protein 1 (CREBI) M34356 | P16220; P21934 | |
| | transcription initiation factor lID; TATA-box factor; | | |
| 429 | TATA sequence-binding protein (TBP) | M34960 | Q16845 |
| | X-ray repair-complementing defective repair in | | |
| 432 | Chinese hamster cells 1 (XRCC1) | M36089 | P18887 |
| | early growth response protein 1 (EGR1); KROX24; | | |
| | nerve growth factor-induced clone A (NGFIA); | | |
| 458 | Z1F268; TIS8; G0S30 | X52541; M62829 | P18146 |
| 459 | transcription factor ETR10l | M62831 | Q03827 |
| | replication protein A 70-kDa subunit (RPA7O; | | |
| | REPAI; RF-A); single-stranded DNA-binding | | |
| 460 | protein | M63488 | P27694 |
| | aryl hydrocarbon receptor nuclear translocator | | |
| 464 | (ARNT); hypoxia-inducible factor 1 beta (HIFi-beta) M69238 | P27540 | |
| | retinoic acid receptor alpha (RAR-alpha; RARA); | X06538; X06614; | P1 0276; Q13440; |
| 466 | PML-RAR protein M73779 | Q13441; Q15156 | |
| | dipeptidyl peptidase IV (DPP IV; DPP4 ); T-cell | | |
| | activation CD26 antigen; TPlO3; adenosine | | |
| 469 | deaminase complexing protein 2 (ADABP; ADCP2) | M74777 | P27487 |
| | clusterin (CLU); complement-associated protein | | |
| | SP-40; complement lysis inhibitor (CLI); | | |
| | apolipoprotein J (APOJ); testosterone-repressed | | |
| | prostate message 2 (TRPM2); sulfated glycoprotein | P10909; P11380; | |
| 470 | 2 (SGP2) M74816 | P11381 | |
| | transcriptional repressor protein yin & yang 1 (YY1); | | |
| | delta transcription factor; DNA-binding protein NF- | | |
| 473 | E1; UCRBP | M76541 | P25490; Q14935 |
| | transcription factor HTF4; transcription factor 12 | | |
| 478 | (TCF12); E-box-binding protein HEB | M80627 | Q99081 |
| | | M84820; X63522; | |
| 487 | retinoic acid receptor beta (RXR-beta; RXRB) | S54072 | P28702; P28703 |
| | activator 1 40-kDa subunit (Al 40-kDa subunit); | | |
| 491 | replication factor C 40-kDa subunit (RFC40); RFC2 | M87338 | P35250 |
| | activator 1 37-kDa subunit; replication factor C 37- | | |
| 492 | kDa subunit (RFC37); RFC4 M87339 | P35249 | |
| | interferon-stimulated gene factor 3 gamma subunit | | |
| | (ISGF3 gamma; ISGF3G); interferon-alpha- | | |
| | responsive transcription factor subunit (IFN-alpha- | | |
| 493 | responsive transcription factor subunit | M87503 | Q00978 |
| | homeobox protein 2.1 (HOX2A); HOXB5; HU1; | | |
| 497 | HHO.C10 | M92299 | P09067; P09069 |
| | tristetraproline (TTP); TIS11; ZFP36; growth factor- | | |
| 498 | inducible nuclear protein 475 (NUP475) | M92843 | P26651 |
| 499 | flil proto-oncogene | M93255 | Q01543 |
| | follicle-stimulating hormone receptor (FSHR); | | |
| 500 | follitropin receptor | M95489 | P23945 |
| | special AT-rich sequence-binding protein 1 | | |
| 505 | (SATB1) | M97287 | Q01826 |
| | neuro epithelioma transforming gene 1 (NEP1; | | |
| 511 | NET1); guanine nucleotide regulatory protein | U02081 | Q12773 |
| 512 | guanine nucleotide regulatory protein tim 1 | U02082 | Q12774 |
| 513 | neu differentiation factor | U02326 | Q12780 |
| 526 | cell division cycle 20 homolog (CDC20) | U05340 | Q12834 |
| | interferon gamma (IFN-gamma) receptor beta | | |
| | subunit; IFN-gamma accessory factor 1 (AE1); IFN- | | |
| 527 | gamma transducer 1 (IFNGT1) | U05875 | P38484 |
| | proto-oncogene tyrosine-protein kinase Ick; p56-Ick; | | |
| | lymphocyte-specific protein tyrosine kinase (LSK); | | |
| 531 | T-cell-specific protein-tyrosine kinase | U07236 | P06239 |
| | epidermal growth factor receptor substrate 15 | | |
| 533 | (EPS15); AF-1 P protein | U07707; Z29064 | P42566 |
| | cytoplasmic nuclear factor of activated T-cells 1 | | |
| 536 | (NF-ATC1) | U08015 | Q12865 |
| | nuclear factor erythroid 2-related factor 1 (NFE2- | | |
| | related factor 1); erythroid-derived 2 nuclear factor- | | |
| | like 1 (NFE2L1; NRF1); transcription factor 11 | | |
| | (TOFu); HBZ17; locus control region factor 1 | | |
| 539 | (LORF1) | U08853 | Q14494; Q12877 |
| 540 | serine kinase | U09564 | Q12890 |
| | cyclin-dependent kinase inhibitor 1 A (CDKN 1A); | | |
| | melanoma differentiation-associated protein 6 | | |
| | (MDA6); CDK-interacting protein 1 (dP1); WAF1; | | |
| 541 | SDI1 | U09579; L25610 | P38936 |
| | Janus tyrosine-protein kinase 3 (JAK3); leukocyte | P52333; Q13259; | |
| 542 | janus kinase (LJAK) | U09607 | Q13260; Q13611 |
| | 45-kDa interleukin enhancer-binding factor 2 (ILF2); | | |
| 543 | 45-kDa nuclear factor (NF45) | U10323 | Q12905 |
| | epidermal growth factor receptor kinase substrate | | |
| 549 | EPS8 | U12535 | Q12929 |
| | caspase 2 (CASP2); neural precursor cell- | | |
| | expressed developmentally down-regulated protein | | |
| 550 | 2 (NEDD2); ICHi | U13021; U13022 | P42575; P42576 |
| | CD151 antigen; platelet-endothelial cell tetraspan | | |
| 558 | antigen 3(PETA3); SEAl | U14650 | P48509; Q14826 |
| 561 | delta-like protein (DLK) | U15979; Z12172 | P80370 |
| 567 | DNA polymerase alpha catalytic subunit (POLA) | X06745 | P09884 |
| 569 | ski oncogene | X15218 | P12755 |
| | neural cell adhesion molecule 1 (NOAMI); CD56 | | |
| | antigen: NCAM 140-kDa isoform (NCAM140) + | | P13591; Q16180; |
| | NCAM phosphatidylinositol-linked isoform; NOAM | | Q15829 + P13592; |
| 571 | 120-kDa isoform (NCAM120) | 571824 + X16841 | P13593 |
| 573 | Wilms' tumor protein (WT33; WT1) | X51630 | P19544 |
| 575 | 120-kDa nucleolar protein 1 (NOLl; NOP120) | X55504 | P46087 |
| 576 | zinc finger X-chromosomal protein (ZFX) | X59738 | P17010 |
| | thioredoxin peroxidase 2 (TDPX2); thioredoxin- | | |
| | dependent peroxide reductase 2; proliferation- | | |
| | associated gene (PAG); natural killer cell enhancing | | |
| 582 | factor A (NKEFA) | X67951 | Q06830; P35703 |
| | macMARCKS; MAROKS-related protein (MRP); | | |
| 583 | MARCKS-like protein (MLP) | X70326 | P49006 |
| | glutamate receptor 5 (GLUR5); lonotropic glutamate | | |
| | receptor kainatel (GRIK1); excitatory amino acid | | |
| 604 | receptor 3 (EAA3) | L19058 | P39086; Q13001 |
| 608 | inhibinalpha(INHA) | M13981 | P05111 |
| | beta 2 adrenergic receptor (ADRB2; ADRB2R; | P07550; Q14823; | |
| 610 | B2AR) | M15169 | Q13714 |
| | insulin-like growth factor-binding protein 3 (IGE- | | |
| 615 | binding protein 3; IGFBP3; IBP3) | M31159; M35878 | P17936 |
| 620 | cellular retinoic acid-binding protein II (CRABP2) | M68867 | P29373 |
| | cell division cycle 25 homolog A (CDC25A); M- | | |
| 633 | phase inducer phosphatase 1 (MPh) | M81933 | P30304 |
| 634 | GuS-specific cyclin 03 (GCND3) | M92287 | P30281 |
| | purine-rich element-binding protein A (PURA); | | |
| | purine-rich single-stranded DNA-binding protein | | |
| 635 | alpha (PUR-alpha) | M96684 | Q00577 |
| 637 | DNA topoisomerase 1 (TOP 1) | J03250 | P11387; Q9UJN0 |
| 638 | DNA topoisomerase II alpha (TOP2A) | J04088 | P11388 |
| | 6-O-methylguanine-DNA methyltransferase | | |
| | (MGMT); methylated-DNA-protein-cysteine | | |
| 640 | methyltransferase | M29971 | P16455 |
| | xeroderma pigmentosum group D complementing | | |
| | protein (XPD); X-ray repair-complementing | | |
| | defective repair in Chinese hamster cells 2 | | |
| 642 | (XRCC2) | X52221 | P18074 |
| 644 | rafi proto-oncogene | X03484 | P04049 |
| 645 | glutathione reductase (GSR) | X15722 | P00390 |
| | | | P00519; Q16133; |
| 646 | c-abl proto-oncogene | M14752 | Q13869; Q13870 |
| | cyclin Dl (CCND 1); parathyroid adenomatosis | | |
| 647 | protein 1 (PRADI) | X59798 | P24385 |
| | microsomal glutathione 5-transferase 1 (MGSTI); | | |
| 648 | GST12 | J03746 | P10620 |
| | excision repair cross-complementing rodent repair | | |
| 649 | deficiency complementation group 1 (ERC01) | M13194 | P07992 |
| | glutathione S-transferase mul (GSTMI; GST1); HB | | |
| 651 | subunit 4; GTH4 | X68676; S01719 | P09488 |
| | glutathione S-transferase pi (GSTP1); GST3; fatty | | |
| 652 | acid ethyl ester synthase III (FAEES3) | X08058; M24485 | P09211 |
| | glutathione S-transferase A1 (GTH1; GSTA1); HA | | |
| 653 | subunit 1; GST-epsilon | M25627 | P08263; Q14750 |
| 654 | cyclin D2 (CCND2) | M90813; 013639 | P30279; Q13955 |
| | ras homolog gene family member A (RHOA; | | |
| | ARHA); aplysia ras-related homolog 12 (ARH12; | | |
| 657 | RHOH12) | L25080 | P06749 |
| 658 | glutathione peroxidase 1 (GSHPX1; GPX1) | Y00483; M21304 | P07203 |
| | | | Z23115; L20121; |
| 660 | apoptosis regulator bcl-x | L20122 | Q07817; Q92976 |
| 661 | cytosohic superoxide dismutase 1 (5001) | K00065; X02317 | P00441 |
| | mutL protein homologi (MLHI; hMLHI); colon | | |
| 662 | cancer nonpolyposis type 2 protein (COCA2) | U07418 | P40692 |
| | UV excision repair protein RAD23 homolog B | | |
| | (hHR23B); xeroderma pigmentosum group C | | |
| | repair-complementing complex 58-kDa protein | | |
| | (XPC repair-complementing complex 58-kDa | | |
| 667 | protein) | D21090 | P54727 |
| | UV excision repair protein RAD23 homolog A | | |
| 668 | (RAD23A; hHR23A) | Q21235 | P54725 |
| 670 | cyclin H (CCNH); M015-associated protein | U11791; U12685 | P51946 |
| 671 | glutathione S-transferase theta 1 (GSTT1) | X79389 | P30711; Q00226 |
| 675 | integrin beta 5 (ITGB5) | J05633 | P18084 |
| | integrin alpha E (ITGAE); 00103 antigen; mucosal | | |
| 677 | lymphocyte antigen 1 alpha (hMLI) | L25851 | P38570 |
| | integrin beta 2 (ITGB2); cell surface adhesion | | |
| | glycoproteins LFA-1/CR3/p150,95 beta subunit; | | |
| | CD18 antigen; complement receptor 03 beta | | |
| 679 | subunit | M15395 | P05107; Q16418 |
| | integrin beta 1 (ITGB1); fibronectin receptor beta | | |
| | subunit (FNRB); very late antigen 4 beta subunit | | |
| 688 | (VLA4); CD29 antigen | X07979 | P05556 |
| | | | P23229; Q14646; |
| 689 | integrin alpha 6 (ITGA6); CD49F antigen; VLA6 | X53586; X59512 | Q16508; Q08443 |
| 692 | integrin alpha 7B (IGA7B) | X74295 | Q13683 |
| | 40-kDa heat shock protein 1 (HSPFI); HSP40; | | |
| 695 | DNAJB1 | Q49547 | P25685 |
| 698 | transcription factor Spi (TSFP1) | J03133 | P08047 |
| 701 | protein kinase C theta (PKC-theta) | L07032 | Q04759 |
| | mitogen-activated protein kinase 8 (MAPK8; | | |
| | PRKM8); c-jun N-terminal kinase 1 (JNKI); stress- | P45983; Q15709; | |
| 702 | activated protein kinase gamma (SAPK-gamma) | L26318 | Q15712; Q15713 |
| | cyclin-dependent kinase 4 inhibitor 2 (CDK4I; | | |
| | CDKN2); p16-INK4; multiple tumor suppressor 1 | | |
| 703 | (MTS1) | L27211 | P42771; Q15191 |
| | mitogen-activated protein kinase p38 (MAP kinase | | |
| | p38); cytokine suppressive anti-inflammatory drug- | | |
| | binding protein (OSAID binding protein; CSBP); | Q16539; Q14084; | |
| 704 | MAX-interacting protein 2 (MX12) | L35253; L35263 | Q13083 |
| | mitogen-activated protein kinase kinase 3 (MAP | | |
| | kinase kinase 3; MAPKK3; MAP2K3; PRKMK3); | | |
| 705 | MAPKIERK kinase 3 (MEK3) | L36719 | P46734 |
| | mitogen-activated protein kinase kinase 4 (MAP | | |
| | kinase kinase 4; MKK4; PRKMK4); c-jun N-terminal | | |
| | kinase kinase 1 (JNKK1); JNK-activating kinase 1; | | |
| 706 | SAPKIERK kinase 1 (SERK1; SEK1) | L36870 | P45985 |
| 708 | N-myc proto-oncogene | M13228 | P04198 |
| | retinoblastoma-associated protein 1 (RB1); | | |
| 709 | phosphoprotein 110 (ppl 10) | M15400 | P06400; P78499 |
| 710 | c-yes proto-oncogene; YESI | M15990 | P07947 |
| 714 | G2/mitotic-specific cyclin Bi (CONBI) | M25753 | P14635 |
| 715 | protein kinase C beta 1 (PRKCB1) | X06318 | P05771 |
| | cAMP-dependent protein kinase type II beta | | |
| 716 | regulatory subunit (PRKAR2B; PKR2) | M31158 | P31323 |
| | heat shock 60-kDa protein (HSP60); HSPDI; 60- | | |
| | kDa chaperonin; mitochondrial matrix protein P1; | | |
| 717 | p60 lymphocyte protein; HUCHA60; GroEL protein | M34664 | P10809 |
| 719 | growth arrest & DNA damage-inducible protein | M60974 | P24522 |
| | (GADD45); DNA damage-inducible transcript 1 | | |
| | (DDIT1) | | |
| | cAMP-dependent protein kinase type 1 beta | | |
| 721 | regulatory subunit (PRKAR1B) | M65066 | P31321 |
| | | | P24864; Q14091; |
| 723 | G1/S-specific cyclin E (CONE) | M73812 | Q92501 |
| | | | P25054; Q15162; |
| 724 | adenomatous polyposis coli protein (APO); DP2.5 | M74088; M73548 | Q15163 |
| | mitogen-activated protein kinase 1 (MAP kinase 1; | | |
| | MAPK1; PRKM 1); extracellular signal-regulated | | |
| 727 | kinase 2 (ERK2) | M84489 | P28482 |
| 730 | Sp2 protein | M97190 | Q02086 |
| 731 | Sp3 protein | M97191 | Q02447 |
| | growth arrest & DNA damage-inducible protein 153 | | |
| | (GADDIS3); DNA damage-inducible transcript 3 | | |
| 732 | (DDIT3); C/EBP homologous protein (CHOP) | S40706; S62138 | P35638 |
| | receptor (TNFRSF)-interacting serine-threonine | | |
| 741 | kinase 1 (RIPKI); receptor-1nteracting protein (RIP) U25994; U50062 | Q13546; Q13180 | |
| | DNA-activated protein kinase catalytic polypeptide | | |
| | (PRKDC); DNA-dependent protein kinase 1 | | |
| | (DNAPK1); hyper-radiosensitivity of murine SCID | | |
| | mutation complementing protein 1 (HYRC1); | Q13337; P78527; | |
| 745 | XRCC7 | U35835; U47077 | Q14175 |
| | cyclin-dependent kinase 4 inhibitor 2D (CDKN2D); | | |
| 746 | p19-INK4D | U40343; U20498 | P55273; Q13102 |
| 748 | breast cancer type 2 susceptibility protein (BROA2) | U43746 | P51587 |
| 749 | G2/mitotic-specific cyclin G1 (CCNG1; CYOGI) | U47413 | P51959 |
| 753 | cyclin A1 (CONA1) | U66838 | P78396 |
| | 90-kDa heat-shock protein A (HSP90A); HSP86; | | |
| 755 | HSPCA | X07270 | P07900 |
| | cAMP-dependent catalytic protein kinase alpha | | |
| 756 | (PRKAC-alpha; PRKACA) | X07767 | P17612 |
| | fos-related antigen 2 (FRA2); fos-like antigen 2 | | |
| 758 | (FOSL2) | X16706 | P15408 |
| 760 | ezrin; cytovillin 2; villin 2 (VIL2) | X51521 | P15311; P23714 |
| | 27-kDa heat shock protein (HSP27); stress- | | |
| | responsive protein 27 (SRP27); estrogen-regulated | | |
| 761 | 24-kDa protein; HSPB1 | X54079 | P04792 |
| 764 | junD proto-oricogene | X56681 | P17535 |
| 765 | c-src kinase (CSK); protein-tyrosine kinase cyl | X59932 | P41240 |
| | mitogen-activated protein kinase 3 (MAP kinase 3; | | |
| | MAPK3; PRKM3); extracellular signal-regulated | | |
| 766 | kinase 1 (ERKI); ERT2 | X60188 | P27361 |
| | | | X56134; M14144; |
| 767 | vimentin (VIM) | Z19554 | P08670 |
| | mitogen-activated protein kinase 6 (MAP kinase 6; | | |
| | MAPK6; PRKM6); p97-MAPK; extracellular signal- | | |
| 769 | regulated kinase 3 (ERK3) | X80692 | Q16659 |
| 770 | fas-activated serine/threonine kinase (FAST) | X86779 | Q14296 |
| | beta 1 catenin (CTNNB); cadherin-associated | | |
| 771 | protein | X87838; Z19054 | P35222 |
| | menage a trois 1 protein (MNAT1; MAT1); CDK- | | |
| | activating kinase assembly factor (OAK assembly | | |
| 773 | factor); CDK7/cyclin H assembly factor | X92669 | P51948 |
| | mitogen-activated protein kinase 9 (MAP kinase 9; | P45984; Q15708; | |
| 779 | MAPK9; PRKM9); c-jun N-terminal kinase 2 (JNK2) L31951 | 015710; 015711 | |
| 780 | protein-tyrosine phosphatase 1 E | L34583 | Q15674 |
| | caspase 1 (CASP1); IL1-beta convertase (IL1BC); U13699; M87507; | | |
| 783 | IL1-beta-converting enzyme (ICE) | X65019 | P29466 |
| | BCL2/adenovirus E1B 19-koa-interacting protein 3 | | |
| 786 | (BNIP3; NIP3) | U15174 | Q12983; 014620 |
| 787 | caspase 6 (CASP6); MCH2-alpha; MCH2-beta | U20536; U20537 | P55212 |
| | | U23765; U16811; | |
| 788 | BCL2 homologous antagonist/killer 1 (BAK1) | X8421 3 | 016611; 092533 |
| | caspase 4 (GASP4); GASPS; ICH2 cysteine | | |
| | protease; ICH3; TX protease; TY protease; | | |
| 789 | ICE(REL) III | U28014; U28015 | P49662; P51878 |
| | mitogen-activated protein kinase 10 (MAP kinase | | |
| | 10; MAPK10; PRKM10); MAP kinase p493F12; c- | | |
| 791 | jun N-terminal kinase 3 alpha 2 (JNK3A2) | U34819; U07620 | P53779; Q15707 |
| | caspase 7 (CASP7); MCH3; ICE-like apoptotic | | |
| 792 | protease 3 (ICE-LAP3); GMH-1 | U37448 | P55210; 013364 |
| | inhibitor of apoptosis proteini (HIAP1; API1) + IAP | | |
| | homolog C; TNFR2-TRAF signaling complex | | |
| 793 | protein 1; MIHO | U45878 + U37546 | Q13489; Q16628 |
| | inhibitor of apoptosis protein 2 (hIAP2; IAP2); IAP | | |
| | homolog B; TNFR2-TRAF signaling complex | | |
| 794 | protein 2; MIHC | U45879; U37547 | Q13490; Q16516 |
| | inhibitor of apoptosis protein 3 (API3; IAP3); X- | | |
| | linked inhibitor of apotosis protein (X-linked IAP; | | |
| 795 | XIAP); IAP-like protein (HILP) | U45880; U32974 | P98170 |
| | caspase 10 (CASP10); MCH4; ICE-like apoptotic | | |
| 799 | protease 4 (ICE-LAP4); FADO-like ICE 2 (FLICE2) | U60519 | Q92851; Q99845 |
| | caspase 8 (CASP8); MORTI -associated CED3 | | |
| | homolog (MACH); FADO-homologous IGE/CED-3- | U60520; U58143; | Q14790; Q15780; |
| 800 | like protease (FADO-like ICE; FLICE); MCH5 | X98172 | Q15806 |
| 801 | interferon regulatory factor 1 (IRF1) | X14454 | P10914 |
| 802 | FAN protein | X96586 | Q92636 |
| 808 | zinc-finger DNA-binding protein | Q45132 | Q13029 |
| | 5-hydroxytryptamine receptor 3A receptor (HTR3A); | | |
| 809 | serotonin receptor | D49394 | P46098 |
| 812 | CDC-like kinase 2 (CLK2) | L29216 | P49760 |
| 813 | CDC-like kinase 3 (CLK3) | L29220 | P49761 |
| 814 | CDC-like kinase 1 (GLK1) | L29222 | P49759 |
| 817 | heat shock 70-kDa protein 1 (HSP70.1; HSPA1) | M11717 | P08107; P19790 |
| 819 | insulin-like growth factor 1 (IGF1); somatomedin C | M27544; M37484 | P01343; 014620 |
| | protein C inhibitor (PROd; PCI); plasma serine | | |
| | protease inhibitor; plasminogen activator inhibitor 3 | | |
| 822 | (PLANH3; PAI3) | M68516; J02639 | P05154 |
| | wee1Hu 00K tyrosine 15-kinase; wee-I-like protein | | |
| 829 | kinase | U10564 | P30291 |
| | DNA damage repair & recombination protein 52 | | |
| 830 | homolog (RAD52) | U12134 | P43351; Q13205 |
| 834 | 3′5′-cAMP phosphodiesterase HPDE4A6 | U18087 | P27815 |
| 835 | CD40 receptor-associated factor 1 (CRAF1) | U21092 | Q13114 |
| 838 | CBL-B | U26710 | Q13191 |
| | 90-kDs TATA box-binding protein-associated factor | | |
| | RNA polymerase III C (TAF3C); transcription factor | | |
| 840 | TFIIIB 90-kDa subunit (TFIIIB90) | U28838 | Q13223 |
| | transcription initiation factor lID 31-kDa subunit | | |
| | (TFIID); TATA-box-binding protein-associated factor | | |
| | RNA polymerase II G 32-kDa subunit (TAFII32; | | |
| 841 | TAF2G); TAFII31 | U30504 | Q16594 |
| | cytoplasmic dynein light chain 1 (hDLC1; DLC1); | | |
| | protein inhibitor of neuronal nitric oxide synthase | | |
| 843 | (PIN) | U32944 | Q15701 |
| 845 | ataxia telangiectasia mutated protein (ATM) | U33841 | Q13315 |
| | erythrocyte urea transporter (UTE; UT1); solute | | |
| | carrier family 14 member 1 (SLC14A1); HUT11; | | |
| 846 | RACH1 | U35735 | Q13336 |
| | mitogen-activated protein kinase kinase 6 (MAP | | |
| | kinase kinase 6; MAPKK6; MAP2K6; PRKMK6); | | |
| 849 | MAPK/ERK kinase 6 (MEK6); SAPKK3 | U39657 | P52564 |
| 851 | C-1 protein; prefoldin 4 (PFDN4; PFD4) | U41816 | Q92779 |
| | ubiquitin-conjugating enzyme E21 (UBE21); UBC9 | | |
| 855 | homolog | U45328 | P50550; Q15698 |
| | mothers against decapentaplegic homolog 1 | | |
| | (MADHI; SMAD1); TGF-beta signaling protein 1 | | |
| 859 | (BSP1) | U57456 | Q15797 |
| 860 | BCL2-like 2 (BCL2L2); BCL-W; K1AA0271 | U59747 | Q92843 |
| 863 | neogenin | U61262 | Q92859 |
| | DNA damage repair & recombination protein 50 | | |
| 864 | homolog (RAD50) | U63139 | Q92878 |
| | cytoplasmic antiproteinase 3 (CAP3); protease | | |
| 867 | inhibitor 19 (P19) | U71364 | P50453 |
| | DNA ligase IV (LIG4); polydeoxyribonucleotide | | |
| 869 | synthase | X83441 | P49917 |
| | DNA ligase III (LIG3); polydeoxyribonucleotide | | |
| 870 | synthase | X84740 | P49916 |
| | deoxyribonuclease I-like protein 1 (DNase1L1; | X90392; L40817; | |
| 871 | DNL1L); DNase X | U06846 | P49184 |
| | cyclin-dependent kinase inhibitor 3 (CDKN3); | | |
| | CDK2-associated dual-specificity phosphatase; | | |
| | kinase-associated phosphatase (KAP); cyclin- | | |
| 876 | dependent kinase interacting protein 2 (CIP2); | L25876 | Q16667; Q99585 |
| | cyclin-dependent kinase interactor 1 (CDI1) | | |
| | Y chromosome variable charge protein (VOY); | | |
| | testis-specific basic protein on Y chromosome 1 | | |
| AH002 | (BPY1) | AF000979 | O14598 |
| AH019 | RAN-binding protein 1 (RANBP1) | D38076 | P43487 |
| | ubiquitin protein ligase E3A (UBE3A); papilloma | | |
| AH038 | virus E6-associated protein (EPVE6AP; E6AP) | L07557 | Q05086; Q93066 |
| | phosphodiesterase 7A (PDE7A); high-affinity | | |
| | cAMP-specific 3,5′-cyclic phosphodiesterase | | |
| AH041 | (HCP1) | L12052 | Q13946; O15380 |
| AH047 | skeletal muscle troponin 1 fast twitch 2 (TNN12) | L21 715 | P48788 |
| AH051 | ADP-ribosylation factor-like 1 (ARL1) | L28997 | P40616; P80417 |
| AH054 | type 1 inositol 1,4,5-triphosphate receptor (ITPR1) L3801 9 | Q14660 | |
| | ADP-ribosylation factor 4-like protein (ARF4L); | | |
| AH055 | ADP-ribosylation factor-like 6 protein (ARL6) | L38490 | P49703 |
| AH061 | angiogenin (ANG); RNaseAfamily5 (RNase5) | M11567 | P03950 |
| AH063 | lamin C | M13451 | P02546 |
| AH064 | related ras oncogene homolog (RRAS) | M14949 | P10301 |
| | protein phosphatase 3 catalytic subunit alpha | | |
| AH070 | (PPP3CA); PPP2B; calcineurin A alpha (CALNA) | M29550 | Q08209 |
| | nuclear receptor subfamily 2 group C member 1 | | |
| AH071 | (NR2C1); TR2 nuclear hormone receptor; TR2-11 | M29960 | Q15625 |
| | dolichyl-phosphate mannosyltransferase | | |
| | polypeptide 2 regulatory subunit (DPM2); dolichol | | |
| AH0735 | phosphate-mannose synthesis protein 2 | AB013361 | 094777 |
| AH0774 | homolog of shkl kinase-binding protein 1 (SKB1) | AF015913 | Q14744 |
| AH07B | ADP ribosylation factor 4 (ARF4) | M36341 | P18085; P21371 |
| AH079 | histone 2AZ (H2AZ) | M37583 | P17317 |
| | PTPRF-interacting protein-binding protein 2 | | |
| AH0809 | (PPFIBP2); liprin beta 2 | AF034803 | Q75337 |
| | SH3 domain-binding glutamic acid-rich-like protein | | |
| AH0825 | (SH3BGRL) | AF042081 | Q75368 |
| | Y-encoded testis-specific-like protein (TSPY-like | | |
| AH0827 | protein; TSPYL) | AF042181 | Q75885 |
| | palmitoylated membrane protein 1 (MPP1); | | |
| | erythrocyte membrane protein 55 (EMP55); | | |
| | palmitoylated erythrocyte membrane protein | | |
| AH086 | (PEMP) | M64925 | Q00013 |
| | voltage-dependent calcium channel alpha 2/delta | | |
| | subunit 1 (CACNA2D1); malignant hyperthermia | | |
| AH090 | susceptibility protein 3 (MHS3) | M76559 | P54289 |
| AH0900 | SYT-interacting protein (SIP) | AF080561 | Q75932 |
| AH0906 | heat shock 40-kDa protein 3 (HSP40-3) | AF088982 | Q75953 |
| | solute carrier family 7 member 7 (SLC7A7); y + L- | | |
| AH0939 | type amino acid transporter 1 (Y + LAT1) | AJ130718 | Q95984 |
| AH094 | skeletal muscle actinin alpha 3 (ACTN3) | M86407 | Q08043 |
| AH0944 | C4.4-like protein | AJ223603 | none |
| | nucleosome assembly protein 1-like 1 (NAPI- | | |
| AH095 | related protein) M86667 P55209 | | |
| | osteoblast-specific factor 2 (OSF2); fasciclin I-like | | |
| AH0951 | protein | D13665 | Q15063; Q15064 |
| | U2 small nuclear ribonucleoprotein auxiliary factor | | |
| AH0962 | small subunit 1 (U2AFIRS1) | D49676 | Q15695; Q13570 |
| AH0965 | transducer of erbB2 2 (TROB2; TOB2); TOB4 | D64109 | Q14106 |
| AH097 | lamin B2 (LMNB2) | M94363 | Q14734 |
| | germline microsatellite sequence (human brain | | |
| AH0998 | cDNA library) | L35592 | none |
| AH1001 | asialoglycoprotein receptor 1 (ASGR1) | M10058 | P07306 |
| AH1002 | cellular retinol-binding protein 1 (RBP1; CRBP1) | M11433 | P09455 |
| AH102 | nuclear autoantigenic sperm protein (NASP) | M97856 | P49321 |
| AH1038 | POM121 homolog & ZP3 fusion protein (POMZP3) | U10099 | Q12903 |
| AH1059 | zeste-white 10 homolog (ZW10; hZW10) | U54996 | Q43264 |
| | solute carrier family 12 member 4 (SLC12A4); | | |
| AH1060 | potassium-chloride cotransporter 1 (KCC1) | U55054 | Q13953 |
| AH110 | alpha 1 syntrophin | S81737 | Q16438 |
| | growth hormone receptor (GH receptor; GHR); | | |
| AH1112 | serum-binding protein | X06562 | P10912 |
| | 60S ribosomal protein L3 (RPL3); HIV-1 TAR RNA- | | |
| AH1123 | binding protein B (TARBP-B) | X73460 | P39023; Q15548 |
| AH1132 | sperm mitochondrial capsule selenoprotein (MCS) | X89960 | P49901 |
| AH1146 | kinesin-like protein 2 (KIF2) | Y08319 | O00139 |
| AH1187 | BAI1-associated protein 1 | AB010894 | Q75085 |
| AH1193 | protein kinase | AB015718 | Q94804 |
| AH1215 | RAD51-interacting protein (PIR51) | AF006259 | Q43403 |
| AH1223 | DNAJ protein | AF012106 | Q14716 |
| AH1248 | alpha 1,2-mannosidase IB | AF027156 | Q60476 |
| AH1254 | timing protein CLK-1 | AF032900 | none |
| | NADH dehydrogenase (ubiquinone) 1 alpha | | |
| | subcomplex 2 (NDUFA2); NADH-ubiquinone | | |
| AH1272 | oxidoreductase B8 subunit | AF047185 | Q43678 |
| | retinal short-chain dehydrogenase/reductase 1 | | |
| AH1292 | (RETSDR1) | AF061741 | Q75911 |
| AH1295 | KEOS protein | AF064605 | Q60737 |
| AH1303 | heat shock factor-binding protein 1 (HSBP1) | AF068754 | Q75506 |
| AH131 | deleted in azoospermia protein (DAZ); SPGY | U21663 | Q13117 |
| AH1318 | sperm-associated antigen 6 (SPAG6); REPROSA1 | AF079363 | Q75602 |
| AH1327 | HOMER3 | AF093265 | Q95350 |
| | glucocerebrosidase pseudogene [alternatively | | |
| AH1355 | spliced] | D13287 | none |
| AH1359 | estrogen-responsive finger protein (EFP) | D21205 | Q14258 |
| AH1381 | intestinal trefoil factor 3 (TFF3; ITF; hITF) | L08044 | Q07654 |
| AH1385 | amino acid transport-related protein | L11696 | Q15295 |
| AH140 | membrane copper amine oxidase; vascular | U39447 | Q16853 |
| | adhesion protein 1 (VAP1); HPAO | | |
| | glutamine-fructose-6-phosphate transaminase 1 | | |
| AH1416 | (GFPT1; GFAT) | M90516 | Q06210 |
| AH1436 | ki nuclear autoantigen | U11292 | Q12920 |
| | centromere protein A (CENPA); centromere | | |
| AH1438 | autoantigen A | U14518 | P49450 |
| | cytosolic brainched-chain amino acid | | |
| AH1443 | aminotransferase (BCAT); ECA39 protein | U21551 | P54687 |
| AH148 | SMOY | U52191 | Q92809 |
| | HS1-binding protein; HSI -associated protein X-1 | | |
| AH1482 | (HAX1) | U68566 | O00165 |
| | erythrocyte membrane protein band 7.2 (EPB72); | | P27105; Q14087; |
| AH1535 | stomatin | X60067 | Q15609 |
| | DiGeorge syndrome critical region 6 protein | | |
| AH1552 | (DGCR6) | X96484 | Q14129 |
| | nucleosome assembly protein 1-like 4; nucleosome | | |
| AH161 | assembly protein 2 (NAP2) | U77456 | Q99733 |
| AH168 | histone 2AX (H2AX) | X14850 | P16104 |
| | alpha centractin; centrosome-associated actin | | |
| AH184 | homolog; actin-RPV 1 (ARP1) | X82206 | P42024 |
| | rev-interacting protein (RIP); rev/rex activation | | |
| AH190 | domain-binding protein | X89478 | P52594; Q15277 |
| | acid lysosomal sphingomyelin phosphodiesterase 1 | | |
| AH2014 | (SMPD1); acid sphingomyelinase | X59960 | P17405; P17406 |
| | | | Q92572; O00721; |
| | | | O00647; O00727; |
| AH215 | clathrin coat assembly protein-like protein | D63643 | Q00676 |
| | DNA-directed RNA polymerase II 33-kDa | | |
| AH225 | polypeptide (RPB33) | J05448 | P19387; Q15161 |
| AH228 | RD protein | L03411 | P18615 |
| AH234 | carbonic anhydrase IV; carbonate dehyratase IV | M83670 | P22748 |
| | serine-rich domain RNA-binding protein SI | | |
| AH245 | (RNPS1) | L37368 | Q15288 |
| AH252 | U2 small nuclear ribonucleoprotein B | M15841 | P08579 |
| AH254 | alpha-2-HS-glycoprotein; fetuin; alpha-2-Z-globulin | M16961 | P02765; Q14962 |
| AH267 | fibrillarin; 34-kDa nucleolar scleroderm antigen | M59849 | P22087 |
| AH283 | fibrillin 2 | U03272 | P35556 |
| | thioredoxin peroxidase A0372; thioredoxin- | | |
| | dependent peroxide reductase A0372; antioxidant | | |
| AH296 | enzyme 372 (A0E372) | U25182 | Q13162 |
| AH298 | RNA-binding motif protein 3 (RBM3); IS1-RNPL | U28686 | P98179 |
| AH306 | C-terminal-binding protein 1 | U37408 | Q13363 |
| | postmeiotic segregation increased homolog 2-like | | |
| AH310 | protein 1 (PMS2L1); PMS3 | D38499 | Q16530 |
| AH311 | beta2-syntrophin | U40572 | Q13425 |
| AH332 | Ul small nuclear ribonucleoproteinG (U1C) | X12517 | P09234 |
| | lupus LA ribonucleoprotein; Sjogren syndrome type | | |
| AH334 | B antigen (SSB) | X13697 | P05455 |
| | heterogeneous nuclear ribonucleoprotein L | | |
| AH336 | (HNRNPL) | X16135 | P14866 |
| | complement factor B; 03/CS convertase; properdin | | |
| | factor B; glycine-rich beta glycoprotein (GBG); | | P00751; O15006; |
| AH348 | PBF2 X72875 Q29944 | | |
| AH352 | poly(C)-binding protein 2; HNRNP-E2 | X78136 | Q15366 |
| | polyadenylate-binding protein 1 (poly(A)-binding | | |
| AH365 | protein 1; PABP1) | Y00345 | P11940; Q93004 |
| | proteasome epsilon subunit; proteasome subunit X; | | |
| | proteasome subunit 6; proteasome subunit MBI; | | |
| | PSMB5; macropain epsilon subunit; multicatalytic | | |
| AH391 | endopeptidase complex epsilon subunit; LMPX | D29011 | P28074; Q16242 |
| AH398 | proteasome subunit Z | D38048 | Q99436 |
| AH405 | transcription elongation factor S-II (hSIIT1) | D50495 | Q15560 |
| AH410 | 605 ribosomal protein L39 (RPL39) | D79205 | P02404; P39025 |
| | folate receptor beta (FR-beta); fetal/placental folate | | |
| AH416 | receptor; placental folate-binding protein (FBP) | J02876 | P14207 |
| | liver/heart cytochrome c oxidase polypeptide VIII | | |
| AH421 | (GOX8) | J04823 | P10176; P15955 |
| | argininosuccinate synthase; citrulline-aspartate | | |
| AH423 | ligase | X01630 | P00966 |
| AH424 | glutathione 5-transferase mu 5 (GSTM5-5) | L02321 | P46439 |
| AH425 | 26S protease regulatory subunit (P2654) | L02426 | Q03527 |
| | alpha-synuclein; non-A beta component of AD | | |
| AH433 | amyloid (NACP) | L08850 | P37840; Q13701 |
| AH436 | 605 ribosomal protein L7 (RPL7) | L16558 | P18124 |
| | protein-lysine oxidase homolog; lysyl oxidase | | |
| AH440 | homolog; lysyl oxidase-like protein | L21186 | Q08397 |
| AH462 | 405 ribosomal protein 517 (RPSI7) | M13932 | P08708 |
| AH464 | uroporphyrinogen decarboxylase (UROD) | M14016 | P06132; Q16883 |
| | glutamine-hydrolyzing asparagine synthetase; TS11 | | |
| AH480 | cell cycle control protein | M27396 | P08243; P08184 |
| | peptidyl-glycine alpha-amidating monooxygenase | | |
| AH487 | (PAM) | M37721 | P19021 |
| | tripeptidyl peptidase II (TPP I1); tripeptidyl | | |
| AH488 | aminopeptidase | M55169 | P29144 |
| | trans-activation-responsive RNA-binding protein | | |
| AH490 | (TAR RNA-binding protein) | M60801 | Q15633; Q12878 |
| | peptidyiprolyl cis-trans isomerase B (PPIase; PPIB); | | |
| | rotamase; cyclophilin B (CYPB); 5-cyclophilirt | | |
| AH491 | (SCYLP) | M60857 | P23284 |
| AH511 | 605 ribosomal protein L24 (RPL24) | M94314 | P38663 |
| AH513 | lysyl hydroxylase | M98252 | none |
| AH515 | transglutaminase | M98479 | none |
| AH523 | 26S proteasome subunit S5B; KIAA0072; HA1357 | S79862 | Q16401; Q15045 |
| AH539 | zinc finger protein 143; SPH-binding factor | U09850 | P52747; O75559 |
| | histidyl-tRNA synthetase homolog (HISRS); | | |
| AH552 | histidine-tRNA ligase homolog | U18937 | P49590 |
| AH582 | transcription initiation protein SPT4 homolog 1 | U43923 | Q16550; Q62387 |
| AH590 | signal recognition particle 54-kDa protein (SRP54) | U51920 | P13624 |
| | signal recognition particle receptor alpha subunit | | |
| AH607 | (SR-alpha); docking protein alpha (DP-alpha) | X06272 | P08240 |
| | liver cytochrome c oxidase polypeptide VIa | | |
| AH614 | (GOX6A) | X15341 | P12074; 043714 |
| | mitochondrial bifunctional | | |
| | methylenetetrahydrofolate | | |
| AH618 | dehydrogenase/cyclohydrolase | X16396 | P13995 |
| AH619 | cytochrome c oxidase polypeptide Vllc (COX7C) | X16560 | P15954 |
| | mitochondrial erythroid-specific 5-aminolevulinic | | |
| | acid synthase (ALAS-E); delta-aminolevulinate | | |
| AH630 | synthase (delta-ALA synthetase) X56352 | P22557 | |
| AH632 | mitochondrial ATP synthase B chain | X60221 | P24539 |
| | elongation factor 1 alpha 2 (EF1-alpha 2; EF1A2); | | |
| AH650 | statin S1 | X70940 | Q05639; P54266 |
| AH653 | trypsinogen IV B form | X71345 | Q15665 |
| | 18-kDa ubiquitin-conjugating enzyme E2; UBCH7; | | |
| | ubiquitin-protein ligase; ubiquitin carrier protein; | | |
| AH683 | UBCM4 | X92962 | P51966 |
| | dolichyl-diphosphooligosaccharide-protein | | |
| AH690 | glycosyltransferase 63-kDa subunit; ribophorin II | Y00282 | P04844 |
| AH692 | glutamine synthetase; glutamate-ammonia ligase | Y00387 | P15104 |
| AH698 | selenoprotein P | Z11793 | P49908 |
| | annexin II (ANX2); lipocortin II; calpactin 1 heavy | | |
| | subunit; chromobindin 8; protein I; placental | | |
| B1 | anticoagulant protein IV (PAPIV) | D00017 | P07355 |
| | retinoic acid-induced gene E (RIGE); lymphocyte | | |
| | antigen 6 complex locus E (LY6E); thymic shared | | |
| B105 | antigen 1 (TSA1); stem cell antigen 2 (SCA2) | U42376 | Q16553 |
| | interferon regulatory factor 5 (IRF-5) DNA-binding | | |
| B110 | nuclear protein | U51127 | Q13568 |
| | macrophage inflammatory protein 3 beta (MIP3- | Q99731 Q00736 | |
| B117 | beta; MIP3B); EBII ligand chemokine (ELO) | U77180 | Q00697 |
| B120 | CX3C chemokine | U91835 | P78423 Q00672 |
| | homeobox protein D4 (HOXD4); HOX4.2; H0X5.1; | | |
| B128 | HHO C13 | X04706; X17360 | P09016 |
| B133 | CD37 antigen | X14046 | P11049 |
| | intercellular adhesion molecule 2 (ICAM2); CD102 | | |
| B134 | antigen | X15606 | P13598 |
| B135 | interferon regulatory factor 2 (IRF2) | X15949 | P14316 |
| B146 | T-cell differentiation 006 antigen; TP12 | X60992 | P30203 |
| B149 | leukemia inhibitory factor receptor (LIFR) | X61615 | P42702 |
| | T cell receptor beta locus (TRB@); T-cell antigen | | |
| B150 | receptor beta polypeptide (TCRB; TRB) | X63456 | none |
| | myosin heavy chain smooth muscle isoform | | |
| B160 | (SMMHC); MYH11 | X69292 | O00396; P35749 |
| B185 | natural resistance-associated macrophage protein 2 | L37347 | P49281 |
| | (NRAMP2) | | |
| B200 | T-cell surface glycoprotein CD1D; R3G1 | J04142 | P15813 |
| | pre-B-cell leukemia transcription factor-I; | | |
| B201 | homeobox protein pbxl; Homeobox protein prl M86546 | P40424 | |
| | pre-B-cell leukemia transcription factor-2; | | |
| B202 | homeobox protein PBX2 | X59842 | P40425 |
| B207 | nucleolysin TIA-1 | M77142 | P31483 |
| | small inducible cytokine subfamily A member 11 | D49372; Z75669; | P51671; P50877; |
| B211 | (SCYA11); CC chemokine eotaxin | Z75668 | Q92490; Q92491 |
| B218 | homeobox protein A4 (HOXA4); HOXiD; HOXI.4 | M74297 | Q00056 |
| | stathmin; phosphoprotein p19 (PP19); oncoprotein | | |
| | 18 (OP18); leukemia-associated phosphoprotein | | |
| B23 | p18 (LAP18); prosolin; metablastin | J04991 | P16949 |
| B239 | homeobox protein A5 (HOXAS); HOXiC | M26679 | P20719 |
| | | | P17813; Q14248; |
| B24 | endoglin (ENG; END); OD10S antigen | J05481; X72012 | Q14926 |
| | homeobox protein B6 (HOXB6); HOX2B; HOX2.2; | X58431; M30597; | |
| B246 | HU-2 | K02571 | P17509 |
| | sex-determining region Y box-containing gene 4 | | |
| B247 | (SRY box-containing gene 4; SOX4) | X70683; X65661 | Q06945 |
| B250 | thy-1 membrane glycoprotein; CDW90 antigen | M11749 | P04216 |
| B34 | AF-4 protein; fel protein | L13773 | P51825 |
| B42 | tyrosine-protein kinase syk; spleen tyrosine kinase | L28824 | P43405 |
| B53 | myeloperoxidase (MPO) | M19507 | P05164 |
| | | | P06748; P08693; |
| | nucleophosmin 1 (NPM1); nucteolar | | Q12826; Q14115; |
| B56 | phosphoprotein B23; numatrin | M23613 | Q13440; Q13441 |
| | perform 1 (P1); lymphocyte pore forming protein | | |
| B63 | (PFP); cytolysin; PREI | M28393 | P14222 |
| | complement decay-accelerating factor (OAF); CD55 | | |
| B64 | antigen | M31516 | P08174 |
| B65 | B-cell lymphoma 3 protein (BCL3) | M31732 | P20749 |
| | CD81 antigen; 26-kDa target of antiproliferative | | |
| B68 | antibody 1 (TAPA1) | M33680 | P18582 |
| | leukocyte surface CD53 antigen; cell surface | | |
| B69 | glycoprotein 0053; M0X44 | M37033 | P19397 |
| | interleukin 3 receptor beta subunit (IL3R-beta; | | |
| | IL3RB); interleukin 5 receptor beta subunit (IL5R- | | |
| | beta; IL5R); cytokine receptor common beta chain; | | |
| B74 | CSF2RB | M59941 | P32927 |
| | B-cell antigen receptor complex associated protein | | |
| | alpha-chain DE; Ig-alpha (IGA); MB-1 membrane | | |
| | glycoprotein; surface-IgM-associated protein; | | |
| | membrane-bound immunoglobulin associated | | |
| B78 | protein; CD79A antigen | M74721 | P11912 |
| B81 | CD14 antigen | M86511 | P08571 |
| B83 | ferritin heavy polypeptide 1 (FTH1); FTHL6 | M97164 | P02794 |
| B85 | B-cell lymphoma 6 protein (BCL6); zinc finger | U00115 | P41182 |
| | protein 51 (ZNF51); LAZ3 protein | | |
| B92 | AF1q | U16954 | Q13015 |
| | lymphotactin (LTN); cytokine scm-i alpha; | | |
| B97 | lymphotaxin; small inducible cytokine Cl (SCYC1) | U23772 | P47992 |
| | | | Q16363; Q15335; |
| C100 | laminin alpha 4 subunit (laminin A4; LAMA4) | X70904; X91171 | Q14735 |
| | chromatin assembly factor 1 p48 subunit (CAF1 | | |
| | p48 subunit); retinoblastoma-binding protein 4 | | |
| C101 | (RBBP4); RBAP48; msil protein homolog | X74262 | Q09028; P31149 |
| C116 | ras-like small GTPase TTF | Z35227 | Q15669 |
| C126 | NEDD5 protein homolog; DIFF6; KIAA0158 | D63878 | Q15019; Q14132 |
| | serine/threonine-protein kinase receptor R3 | | |
| | (SKR3); activin receptor-like kinase 1 (ACVRL1; | | |
| | ALK1); TGF-beta superfamily receptor type 1 | | |
| C13 | (TSR1) | L17075 | P37023 |
| C130 | collagen X1alpha 1 subunit(COLI1A1) | J04177 | P12107 |
| | bone/cartilage small proteoglycan 1 (PGS 1); | | |
| C131 | biglycan (BGN) | J04599 | P21810; P13247 |
| | E2F dimerization partner 1 (TFDP1); DRTF1 | | |
| C138 | polypeptide 1 (DP1) | L23959 | Q14186 |
| C14 | glial cell line-derived neurotropic factor (GDNF) | L19063 | P39905 |
| C152 | cell division cycle 25 homolog C (CDC25C) | M34065 | P30307 |
| | CDC42 homolog; G25K GTP-binding protein (brain | | |
| C154 | isoform + placental isoform) | M35543 + M57298 | P21181 + P25763 |
| | insulin-like growth factor binding protein 5 (IGF- | | |
| C159 | binding protein 5; IGFBP5) | M65062 | P24593 |
| | neurogenic locus notch protein homolog 1 | | |
| | (NOTCH 1); translocation-associated notch protein | | |
| C160 | (TAN1) | M73980 | P46531 |
| | cadherin-associated protein-related protein (CAPR); | | |
| C162 | alpha 2 catenin (CTNNA2); alpha N catenin | M94151 | P26232 |
| C163 | B-raf proto-oncogene (RAFB1) | M95712 | P15056 |
| C17 | collagen XVIII alpha 1 subunit (COL18A1) | L22548 | P39060 |
| C170 | p21-activated kinase alpha (PAK-alpha; PAK1) | U24152 | Q13153; Q13567 |
| | p21-activated kinase gamma (PAK-gamma; PAK2); | | |
| C171 | PAK65; S6/H4 kinase | U24153 | Q13177; Q13154 |
| | mitogen-activated protein kinase 7 (MAP kinase 7; | | |
| | MAPK7; PRKM7); extracellular signal-regulated | | |
| C172 | kinase 5 (ERKS); BMK1 kinase | U25278 | Q13164; Q16634 |
| | | | P13942; Q13273; |
| C173 | collagen X1alpha 2 subunit (COLl 1A2) | U32169 | Q07751 |
| | mitogen-activated protein kinase P38 beta (MAP | | |
| | kinase P38 beta); stress-activated protein kinase 2 | | |
| C179 | (SAPK2) | U53442 | Q15759; Q00284 |
| | FKBP-rapamycin associated protein (FRAP); | | |
| C18 | rapamycin target protein | L34075 | P42345 |
| | cell division control protein 2 homolog (CDC2); p34 | | |
| C183 | protein kinase; cyclin-dependent kinase 1 (CDK1) | X05360 | P06493 |
| C185 | collagen IV alpha 2 subunit (COL4A2) | X05610 | P08572 |
| | low-density lipoprotein receptor-related protein 1 | | |
| | (LRP); alpha-2-macroglobulin receptor (A2MR); | | |
| C187 | apolipoprotein E receptor (APOER); CD91 antigen | X13916 | Q07954 |
| C188 | collagen III alpha 1 subunit (COL3A1) | X14420 | P02461 |
| C190 | procollagen II alpha 1 subunit (COL2A1) | X16468 | P02458 |
| C191 | collagen IX alpha 1 subunit (COL9A1) | X54412 | P20849 |
| | mitogen-activated protein kinase 4 (MAP kinase 4; | | |
| C195 | MAPK4; PRKM4); ERK3-related protein | X59727 | P31152 |
| | cadherin 3 (CDH3); placental cadherin (P-cadherin; | | |
| C197 | CDHP) | X63629 | P22223 |
| | serine/threonine-protein kinase PCTAIRE 3 | | |
| C199 | (PCTK3) | X66362 | 007002 |
| | serine/threonine-protein kinase POTAIRE 1 | | |
| C200 | (PCTK1) | X66363 | Q00536 |
| | retinoblastoma-like protein 2 (RBL2; RB2); 130-kDa | | |
| C204 | retinoblastoma-associated protein | X74594 | Q08999 |
| | tenascin (TN); hexabrachion (HXB); cytotactin; | | |
| | neuronectin; GMEM; miotendinous antigen; glioma- | | P24821; Q15567; |
| C206 | associated extracellular matrix antigen | X78565; M55618 | Q14583 |
| | mitogen-activated protein kinase 12 (MAP kinase | | |
| | 12; MAPK12; PRKM12); MAP kinase p38 gamma; | | |
| | stress-activated protein kinase 3 (SAPK3); | | |
| C207 | extracellular signal-regulated kinase 6 (ERK6) | X79483 | P53778 |
| | cyclin-dependent kinase 5 activator (CDKS | | |
| | activator); tau protein kinase II 23-kDa subunit; | | |
| C209 | TPKII regulatory subunit | X80343 | Q15078 |
| C21 | alpha-2-macroglobulin (alpha-2-M) | M11313 | P01023 |
| | cadherini (CDH 1); epithelial cadherin (E-cadherin; | | |
| C215 | CDHE); uvomorulin (UVO); CAM 120/80 | Z13009 | P12830; Q14216 |
| | | | Q13752; Q14941; |
| C216 | laminin gamma 2 subunit (LAMC2) | Z15009 | Q02536 |
| C22 | proliferating cyclic nuclear antigen (PCNA); cyclin | M15796; J04718 | P12004 |
| | wingless-related MMTV integration site 13 protein | | |
| C220 | (WNT1 3) | Z71621 | Q93097; 14903 |
| C221 | myeloid cell leukemia protein 1 (MCL1) | L08246 | Q07820 |
| C222 | PDCD2 | S78085 | Q16342 |
| C234 | DAXX | AF015956 | O14747 |
| C238 | hyaluronan receptor (RHAMM) | U29343 | O75330 |
| | platelet glycoprotein IV (GPIV; PAS IV; PAS4); | | |
| C24 | GPIIIB; CD36 antigen | M24795 | P16671 |
| | BCL2 antagonist of cell death (BAD); BCL2L8; | | |
| C240 | BCL2-binding component 6 (BBC6) U66879 | Q92934 | |
| | mitogen-activated protein kinase kinase kinase 3 | | |
| | (MAPKKK3;.MAP3K3); MAPK/ERK kinase kinase 3 | | |
| C250 | (MEK kinase 3; MEKK3) | U78876 | Q99759 |
| C254 | smoothened homolog (SMOH) | U84401 | Q99835 |
| C257 | neurogenic locus notch protein homolog 4 | U95299 | O00306 |
| | (NOTCH4) | | |
| | BCL2-binding athanogene 1 (BAG1); glucocorticoid | | Q99933; Q14414; |
| C259 | receptor-associated protein RAP46 | 583171; Z35491 | O75315 |
| C265 | NIK serine/threonine protein kinase | Y10256 | Q99558 |
| C27 | nidogen (NID); entactin | M30269 | P14543; Q14942 |
| C274 | manic fringe homolog (MFNG) | U94352 | 000587 |
| C277 | c-fos proto-oncogene; G0S7 protein | K00650 | P01100 |
| C282 | plasminogen (PLG) | X05199 | P00747 |
| | mitogen-activated protein kinase kinase 2 (MAP | | |
| | kinase kinase 2; MAPKK2; MAP2K2; PRKMK2); | | |
| C286 | MAPKIERK kinase 2 (MEK2) | L11285 | P36507 |
| C287 | Indian hedgehog (IHH) | L38517 | Q14623 |
| | | | M24857; M38258; |
| C290 | retinoic acid receptor gamma (RARG) | M57707; M32074 | P13631 |
| C292 | v-Ki-RAS2B proto-oncogene (KRAS2) | M54968 | P01118 |
| | akt1 proto-oncogene; rac alpha serine/threonine | | |
| C296 | kinase (RAC-PK-alpha); protein kinase B (PKB) | M63167 | P31749 |
| | rac-beta serinelthreonine kinase (rac-PK-beta); | | |
| C297 | AKT2 | M77198; M95936 | P31751 |
| | casper, a FADD- and caspase-related inducer of | | |
| | apoptosis (CASH-alpha + CASH-beta); FLAME-1; | AF010127; | |
| C304 | FLICE-like inhibitory protein | Y14039; Y14040 | O15519 |
| | ras-related C3 botulinum toxin substrate 1 (RAC1); | | |
| C309 | ras-likeprotein TC25 | M29870; M31467 | P15154 |
| | PRB-binding protein E2F1; retinoblastoma-binding | | |
| | protein 3 (RBBP3); retinoblastoma-associated | | Q01094; Q92768; |
| C313 | protein 1 (RBAP1); PBR3 | M96577 | Q13143 |
| | | U91903; U24163; | O00181; Q92765; |
| C314 | frizzled-related FrzB (FRITZ); frezzled (FRE) | U68057 | Q99686 |
| | mitogen-activated protein kinase kinase 5 (MAP | | |
| | kinase kinase 5; MAPKK5; MAP2KS; PRKMK5); | | |
| C315 | MAPKIERK kinase 5 (MEKS) | U25265 | Q13163 |
| | caspase & RIP adaptor with death domain | | |
| | (CRADD); RIP-associated protein with death | | |
| C318 | domain (RAIDD) | U84388 | P78560 |
| | CD59 glycoprotein; membrane attack complex | | |
| | inhibition factor (MACIF); MAC inhibitory protein | | |
| | (MACIP); MEM43 antigen; protectin; membrane | | |
| | inhibitor of reactive lysis (MIRL); HRF20; IFS | | |
| C32 | antigen | M34671 | P13987 |
| C34 | CD9 antigen; leukocyte antigen MIC3 | M38690 | P21926 |
| C349 | apoptosis-related protein TFARI5 | AF022385 | Q14811 |
| | signal transducer and activator of transcription 1 | | |
| | alpha/beta (STAT1); transcription factor ISGF-3 | | |
| C354 | components p91/p84 | M97935 | P42224 |
| | metalloprotease/disintegrin/cysteine-rich protein | | |
| C358 | (MDC9) | U41766 | Q13443 |
| C365 | seven in absentia homolog | U63295 | Q43269 |
| | glutathione-S-transferase-like protein (GSTTLp28); | | |
| C370 | glutathione transferase omega (GST01) | U90313 | P78417 |
| | Bruton+S tyrosine kinase (BTK); | | |
| | agammaglobulinaemia tyrosine kinase (ATK); B-cell | | |
| C372 | progenitor kinase (BPK) | U10087; X58957 | Q06187 |
| | ephrin type-A receptor 2; epithelial cell kinase | | |
| C385 | (ECK); tyrosine-protein kinase receptor ECK | M59371 M36395 | P29317 |
| | signal transducer & transcription activator 5A | | |
| C388 | (STAT5A);STAT5B | U47686 | P51692; P42229 |
| C48 | collagen XVI alpha 1 subunit (COL16A1) | M92642 | Q07092 |
| C49 | prostate-specific membrane antigen (PSM) | M99487 | Q04609 |
| C54 | DNA mismatch repair protein MSH2 | U04045; L47583 | P43246 |
| | putative RHO/RAC guanine nucleotide exchange | | |
| | factor (rho/rac GEE); faciogenital dysplasia protein | | |
| C56 | 1 (FGD1) | U11690 | P98174 |
| C63 | metastasis-associated protein 1 (MTA1) | U35113 | Q013330 |
| | DNA mismatch repair protein MSH6; mutS alpha | | |
| | 160-kDa subunit; G/T mismatch binding protein | | P52701; Q43706; |
| C77 | (GTMBP; GTBP) | U54777 | O43917 |
| | nucleoside diphosphate kinase A (NDKA); NDP | | |
| | kinase A; tumor metastatic process-associated | | |
| | protein; metastasis inhibition factor NM23 (NM23- | | |
| C88 | H1) | X17620 | P15531 |
| C90 | collagen VI alpha 3 subunit (COL6A3) | X52022 | P12111 |
| C94 | collagen VIII alpha 1 subunit (C0L8A1) | X57527 | P27658 |
| | 40S ribosomal protein 530 (RPS3O); Finkel-Biskis- | | |
| | Reilly murine sarcoma virus ubiquitously expressed | | P35544; Q05472; |
| C97 | gene (FAU; FUB1) | X65923 | Q95261 |
| CA007 | tousled-like kinase 2 (TLK2); PKU-alpha | AB004884 | Q9Y4F7 |
| CA008 | STAT-induced STAT inhibitor 2 | AB004903 | Q14508 |
| CA018 | Janus tyrosine-protein kinase 2 (JAK2) | AF005216 | Q60674 |
| CA027 | apoptotic protease activating factor 1 (APAF1) | AF013263 | Q14727 |
| CA034 | DNA recombination & repair protein HNGS1 | AF022778 | Q43475 |
| | mitogen-activated protein kinase kinase 7 (MAP | | |
| | kinase kinase 7; MAPKK7; MAP2K7; PRKMK7); | | |
| | MAPK/ERK kinase 7 (MEK7); c-jun N-terminal | | |
| CA035 | kinase kinase 2 (JNKK2) | AF022805 | Q14816; Q60452 |
| | cysteine-rich anigogenic inducer 61 (CYR61); | | |
| | insulin-like growth factor-binding protein 10 (IGE- | | |
| CA043 | binding protein 10; IGFBP10); GIG1 | AF031385 | Q00622; Q14934 |
| | cAMP-responsive element modulator 1 alpha | | |
| CA053 | protein (HCREM) | D14825 | Q14501 |
| | MCM2 DNA replication licensing factor; nuclear | | |
| CA057 | protein BM28; KIAA0030 | D21063 | P49736 |
| CA062 | KIAA0078 | D38551 | Q60216 |
| | | | P02593; P99014; |
| | | | P70667; Q61379; |
| CA066 | calmodulin (CALM; CAM) | D45887 | Q61380 |
| CA070 | cysteine protease | 055696 | Q99538 |
| CA087 | fibroblast adenine nucleotide translocator 2 (ANT2) | J02683 | P05141; O43350 |
| CA090 | uroporphyringen III synthase | J03824 | P10746 |
| CA093 | leukocyte lgG Fo receptor (FCGR) | J04162 | P08637 |
| CA103 | interferon-gamma lEE SSP 5111 | L07633 | Q06323 |
| CA111 | aryl hydrocarbon receptor (AH receptor) | L19872 | P35869 |
| CA119 | glutathione S-transferase theta 2 (GSTT2) | L38503 | P30712 |
| CA121 | protein phosphatase 2A B56-alpha | L42373 | Q15172 |
| CA122 | protein phosphatase 2A B56-beta (PP2A) | L42374 | Q15173 |
| | protein phosphatase PP2A 61-kDa regulatory | | |
| CA124 | subunit episilon | L76703 | Q16537 |
| | signal transducer and activator of transcription 4 | | |
| CA125 | (STAT4) | L78440 | Q14765 |
| | eukaryotic translation initiation factor 4E 25-kDa | | |
| CA127 | subunit (EIF4E); mRNA cap-binding protein | M15353 | P06730 |
| | 2-oxoisovalerate dehydrogenase alpha subunit; | | |
| | branched-chain alpha-keto acid dehydrogenase | | |
| CA131 | component alpha subunit (BCKDH El-alpha) | M22221 | P12694 |
| CA134 | elk-1; ets-related proto-oncogene | M25269 | P19419 |
| CA143 | GTP-binding nuclear protein RAN (TC4) | M31469 | P17080 |
| | cAMP-dependent protein kinase beta catalytic | | |
| CA152 | subunit (PKAC-beta; PRKACB) | M34181 | P22694 |
| | cAMP-dependent protein kinase gamma-catalytic | | |
| CA153 | subunit (PKA C-gamma) | M34182 | P22612 |
| CA160 | deoxycytidine kinase | M60527 | P27707 |
| CA167 | complement component 5 (CS) | M65134 | P01031 |
| CA175 | lymphocyte antigen | M81141 | Q30099 |
| | interleukin 16 (IL16); lymphocyte chemoattractant | | |
| CA181 | factor (LCF) | M90391 | Q14005 |
| | serine/threonin protein phosphatase 2B catalytic | | |
| | subunit gamma; calmodulin-dependent calcineurin | | |
| | A subunit gamma; testic-specific calcineurin | | |
| CA186 | catalytic subunit (CAM-PRP catalytic subunit) | S46622 | P48454 |
| CA197 | serinelthreonine protein kinase | U07358 | Q12852 |
| | serine/threonine-protein kinase NEK2; NI MA- | | |
| | related protein kinase 2; NIMA-like protein kinase 1; | | |
| CA204 | HSPK21 | U11050 | P51955 |
| CA206 | 40S ribosomal protein S5 (RPS5) | U14970 | P46782 |
| CA226 | 58-kDa inhibitor of the RNA-activated protein kinase | U28424 | Q13217 |
| CA229 | interleukin 15 receptor alpha subunit | U31628 | Q13261 |
| CA230 | histone deaceytlase 2 (HD2) | U31814 | Q92769 |
| CA234 | TAXI-binding protein 151 (TXBP151) | U33821 | Q13311 |
| | glutathione synthetase (GSH synthetase; GSH-S); | | |
| CA237 | glutathione synthase | U34683 | P48637 |
| CA242 | xanthine dehydrogenase/oxidase | U39487 | P47989 |
| | mothers against decapentaplegic homolog 4 | | |
| CA251 | (DPC4; MADH4; SMAD4) | U44378 | Q13485 |
| CA255 | CREB-binding protein | U47741 | Q92793 |
| CA263 | hint protein; protein kinase C inhibitor 1 (PKCI1) | U51004 | P49773 |
| CA271 | serine/threonine protein kinase KRS2 | U60207 | Q15802 |
| CA276 | preferentially expressed antigen of melanoma | U65011 | P78395 |
| CA277 | mitotic feedback control protein MADP2 homolog | U65410 | Q13257 |
| | branched-chain amino acid aminotransferase | | |
| CA281 | mitochondrial (BCAT(M)) | U68418 | O15382 |
| CA290 | HSIAH2 | U76248 | O43270 |
| CA294 | TSG10I tumor susceptibility protein | U82130 | Q99816 |
| | maleylacetoacetate isomerase (MAA1); glutathione | | |
| CA299 | transferase zeta 1 | U86529 | O43708 |
| | autoimmunogenic cancer/testis antigen NY-ESO-1; | | |
| CA300 | LAG E-1 | U87459 | P78358 |
| | purine nucleoside phosphorylase (PNP); inosine | | |
| CA308 | phosphorylase | X00737 | P00491 |
| CA313 | cathepsin L; major excreted protein (MEP) | X12451 | P07711 |
| | regulator of chromosome condensation; cell cycle | | |
| CA314 | regulatory protein | X12654 | P18754 |
| | ribonucleoside-diphosphate reductase M1 subunit; | | |
| CA328 | ribonucleotide reductase | X59543 | P23921 |
| | CMP-N-acetylneuraminate-beta-galactosamide- | | |
| | alpha-2,6-sialyltransferase; beta-galactoside alpha- | | |
| | 2,6-sialyltransferase (alpha-2,6-ST); | | |
| CA331 | sialyltransferase 1 (SIAT1); B-cell CD75 antigen | X62822 | P15907 |
| | DNA primase small subunit; DNA primase 49-kDa | | |
| CA340 | subunit | X74330 | P49642 |
| | minichromosome maintenance deficient homolog 4 | | |
| CA341 | (MCM4); CDC21 homolog; CDC54 homolog | X74794 | P33991; Q99658 |
| CA350 | thioredoxin reductase | X91247 | Q16881 |
| CA355 | serine/threonine protein kinase | X97630 | Q15524 |
| | secretogranin 1 (SGI; SOG1); chromogranin B | | |
| CA358 | (CHGB) | Y00064 | P05060 |
| | ets domain protein elk-3; NET; SRF accessory | | |
| CA379 | protein 2 (SAP2) | Z36715 | P41970 |
| | alpha-N-acetylneuraminnide alpha-2,8- | | |
| | sialyltransferase; ganglioside GD3/GT3 synthase; | | |
| CA386 | sialyltransferase 8 (SIAT8) | L32867 | Q92185 |
| | glutamate-cysteine ligase regulatory subunit | | |
| CA387 | (GLCLR); gamma-glutamylcysteine synthetase | L35546 | P48507 |
| | estrogen receptor 1 (ESR1); estrogen receptor | | |
| | alpha (ER-alpha; ESRA); fatty acid | M12674; X03635; | P03372; Q14276; |
| CA389 | synthase/estrogen receptor fusion protein (FAS/ER) | U47678; AF172068 | Q13511 |
| | soluble galactose-binding lectin 3 (LGALS3); | | |
| | LGALS2; galectin 3, macrophage galactose-specific | | |
| | lectin 2 (MAC2); galactoside-binding protein | | |
| CA394 | (GALBP) | M35368 | P17931; Q16005 |
| CA397 | LOX (Protein-lysine 6-oxidase, Lysyl-Oxidase) | M94054 | P28300 |
| CA401 | stanniocalcin (STC) | U25997 | P52823 |
| | RAD51 B, RAD51 LI, REC2, R51 H2 (DNA repair | | |
| CA406 | protein RAD 51 Homolog 2) | U92074 | O60914; O15315 |
| CA407 | SAP18 (Sin3 associated polypeptide P18) | U96915 | Q00422 |
| CA414 | RAD51C truncated protein | AF029670 | Q43503 |
| | chromodomain-helicase-DNA-binding protein 1 | | |
| CA419 | (CHD1) | AF006513 | Q14646 |
| CA420 | CDC7-reiated kinase | AF015592 | Q00311 |
| | stratifin (SFN); 14-3-3 protein sigma; epithelial cell | | |
| CA426 | marker protein 1; HMEI | AF029082 | P31947 |
| CA431 | testis-specific protein kinase 1 | D50863 | Q15569 |
| CA438 | KIAA0204 | D86959 | Q92603 |
| CA444 | microtubule-associated protein 1B | L06237 | P46821 |
| CA445 | Von Hippel-Lindau tumor suppressor protein (VHL) | L15409 | P40337 |
| CA446 | thymidylate kinase | L16991 | P23919 |
| | A-raf proto-oncogene serine/threonine-protein | | |
| CA450 | kinase; PKS2 | L24038 | P10398 |
| | importin beta 1 subunit; karyopherin beta 1 subunit; | | |
| CA452 | importin 90; nuclear factor p97 | L38951 | Q14974 |
| | (2′-5′)oligoadenylate synthetase 1 ((2-5′)oligo(A) | | |
| CA459 | synthetase 1; 2-5A synthetase 1) | M11810 | P00973 |
| | eukaryotic translation initiation factor 2 alpha | | |
| CA482 | subunit (EIF2-alpha) | U26032 | none |
| CA487 | betaine-homocysteine S-methyltransferase | U50929 | Q93088 |
| CA488 | mitogen-responsive phosphoprotein DOC2 | U53446 | Q13598 |
| CA498 | MYD88 | U70451 | P78397 |
| CA502 | microsomal glutathione S-transferase 2 (MGST2) | U77604 | Q99735 |
| | transcription factor activating enhancer-binding | | |
| CA506 | protein 2 gamma (AP2-gamma; TFAP2C) | U85658 | Q92754 |
| CA508 | serine kinase SRPK2 | U88666 | P78362 |
| CA510 | growth-arrest-specific protein 2 (GAS2) | U95032 | Q43903 |
| CA513 | hemoglobin alpha subunit | V00491 | P01922 |
| CA518 | trifunctional purine biosynthetic protein adenosine 3 | X54199 | P22102 |
| | major histocompatibility complex class II DM alpha | | |
| CA528 | subunit (HLA-DMA); RING6 | X62744 | P28067 |
| CA532 | cardiac ventricular myosin light chain 2 | X66141 | Q14908 |
| CA533 | adenylosuccinate synthetase; IMP-aspartate ligase | X66503 | P30520 |
| CA540 | uridine phosphorylase (UDRPase; UP) | X90858 | Q16831; Q15362 |
| | neutrophil gelatinase-associated lipocalin (NGAL); | | |
| | 25-kDa alpha-2-microglobulin-related subunit of | | |
| CA546 | MMP9); lipocalin 2; oncogene 24P3 | X99133 | P80188 |
| CA547 | adenine phosphoribosyltransferase (APRT) | Y00486 | P07741 |
| | CCAATfenhancer-binding protein delta (C/EBP | | |
| CU012 | delta); nuclear factor-IL-6 beta protein (NF-IL6-beta) | M83667 | P49716; Q14937 |
| CU038 | DNA (cytosine-5-)-methyltransferase 2 (DNMT2) | AJ223333 | Q14717 |
| CU041 | retinoblastoma-related protein p107 | L14812 | P28749 |
| | ATP-binding cassette subfamily B (MDRITAP) | | |
| | member 1 (ABCB1); multiple drug resistance 1 | P08183; Q12755; | |
| CU043 | protein (MOR1); P glycoprotein 1 (PGY1) | M14758 | Q14812 |
| CU047 | glutathione S-transferase mu 4 (GSTM4) | M96233 | Q03013 |
| | | | P11137; Q99976; |
| CU048 | microtubule-associated protein 2 (MAP2) | U01828 | Q99975 |
| | lysophosphatidic acid G protein-coupled endothelial | Q92633; Q00722; | |
| CU120 | differentiation receptor 2 (EDG2); vzg-1z | U80811 | Q00656; P78351 |
| | TTAGGG repeat binding factor 2 (hTRF2); | | |
| E011 | telomeric DNA-binding protein | AF002999 | Q15554 |
| | DNA polymerase epsilon subunit B; DNA | | |
| E017 | polymerase II subunit B | AF025840 | P56282 |
| | chondroitin sulfate proteoglycan 5 (CSPG5); | | |
| E046 | neuroglycan C (NOC) | AF059274 | Q95196 |
| | serine/threonin-protein kinase PAK-beta; p21 - | | |
| E054 | activated kinase 3 | AF068864 | Q75914 |
| | growth arrest & DNA damage-inducible protein 45 | | |
| E065 | gamma (GADD45 gamma) | AF078078 | none |
| E069 | cyclin D-binding MYB-like protein (hDMP1) | AF084530 | Q9Y222 |
| E075 | ribosomal protein kinase B (RSKB) | AJ010119 | Q75676 |
| | ferrochelatase; protoheme ferro-lyase; heme | | |
| E077 | synthetase | D00726 | P22830 |
| | proteasome (prosome, macropain) subunit alpha | | |
| E081 | type 3 (PSMA3); HC8 | D00762 | P25788 |
| | peripheral myelin protein 22 (PMP22); CD25 | | |
| E083 | protein; SR13 myelin protein | D11428 | Q01453 |
| E096 | photolyase/blue-light receptor homolog | D84657 | Q16526 |
| | protein kinase C substrate 80-kDa protein heavy | | |
| E102 | chain (PKCSH); 80K-H protein | J03075 | P14314 |
| E103 | liver glucose transporter 2 | J03810 | P11168 |
| | serotransferrin (TF); siderophilin; beta-1-metal- | | |
| E118 | binding globulin | M12530 | P02787 |
| | DNA-binding protein A (DBPA); cold shock domain | | |
| E123 | protein A (CSDA) | M24069 | P16989; Q14121 |
| | 26S protease regulatory subunit 6A; TAT-binding | | |
| E126 | protein 1 (TBP1); proteasome subunit p50 | M34079 | P17980 |
| | solute carrier family 9 member 1 (SLC9A1); | | |
| | sodium/hydrogen exchanger 1 (Na + /H+ exchanger | | |
| E130 | 1;NHE1) | M81768 | P19634 |
| E132 | B94 protein | M92357 | Q03169 |
| | RaIGDSB; GTP/GDP dissociation stimulator for a | | |
| E148 | ras-related GTPase (RALGEF) | U14417 | Q12967 |
| E153 | selenium-binding protein | U29091 | Q13228 |
| | methionine aminopeptidase 2 (METAP2); peptidase | | |
| | M2; initiation factor 2-associated 67-kDa | | |
| E154 | glycoprotein | U29607 | P50579 |
| E159 | leptin receptor (LEPR); OB receptor (OBR); FA | U43168 | P48357 |
| E160 | myotubularin | U46024 | Q13496 |
| | sodium/potassium-transporting ATPase beta 3 | | |
| | subunit (ATPB3); sodium/potassium-dependent | | |
| E161 | ATPase | U51478 | P54709 |
| | alpha-1-acid glycoprotein 1 (AGP1); orosomucoid 1 | | |
| E178 | (OMD1) | X02544 | P02763 |
| | inter-alpha-trypsin inhibitor heavy chain H2 (ITI | | |
| | heavy chain H2); inter-alpha-trypsin inhibitor | | |
| E181 | complex component II | X07173 | P19823 |
| | carboxypeptidase H (CPH); CPE; enkephalin | | |
| | convertase; prohormone processing | | |
| E185 | carboxypeptidase | X51405 | P16870 |
| E186 | lactotransferrin; lactoferrin | X53961 | P02788 |
| | major epididymis-specific protein E4 (hE4); | | |
| E188 | epididymal secretory protein E4 | X63187 | Q14508 |
| | inter-alpha-trypsin inhibitor heavy chain H3 (ITI | | |
| E190 | heavy chain H3) | X67055 | Q06033 |
| E193 | tuberin; tuberous sclerosis 2 protein (TSC2) | X75621 | P49815 |
| E196 | translin; recombination hotspot binding protein | X78627 | Q15631 |
| | dipeptidyl-peptidase 1 (DPP-1); cathepsin C; | | |
| E198 | cathepsin J; dipeptidyl transferase | X87212 | P53634 |
| | ATP-binding casette 8 (ABC8); Drosophila white | | |
| E199 | homolog | X91249 | P45844 |
| | phosphatidylinositol-specific phospholipase C beta | | |
| E210 | 3 (PLC-beta 3; PLCB3) | Z16411 | Q01970 |
| E215 | bystin | L36720 | Q13895 |
| E219 | acrosin-trypsin inhibtor II; HUSI II | M91438 | P20155 |
| E220 | emsl oncogene; cortactin (GUN); amplaxin | M98343 | Q14247 |
| | autosomal dominant polycystic kidney disease II | | |
| E228 | (PKD2) | U50928 | Q13563 |
| E230 | organic cation transporter 1 | U77086 | Q15395 |
| E231 | alpha-fetoprotein; alpha-fetoglobulin | V01514 | P02771 |
| E232 | C-reactive protein | X56692 | P02741 |
| E241 | C-ets-2 | J04102 | P15036 |
| | heparin cofactor II (HCI1); protease inhibitor | | |
| E244 | leuserpin 2 (hLS2) | M12849 | P05546 |
| | ALPHA-l-MICROGLOBULIN/INTER-ALPHA- | | |
| | TRYPSIN INHIBITOR LIGHT CHAIN (ITI-LC) | | P02759; P00977; |
| E252 | (PROTEIN HG) (HI-30) (BIKUNIN) | X04494 | P02760 |
| | INTER-ALPHA-TRYPSIN INHIBITOR HEAVY | | |
| | CHAIN HI (ITI HEAVY CHAIN H1) (INTER-ALPHA- | | |
| | TRYPSIN INHIBITOR COMPLEX COMPONENT | | P78455; Q01746; |
| E254 | III) | X63652 | P19827 |
| G014 | PTH/PTHrP receptor | L04308 | Q03431 |
| | prostaglandin E2 receptor EP1 subtype (PTGER1); | | |
| G021 | prostanoid EPI receptor | L22647 | P34995 |
| | guanine nucleotide-binding protein G(K) alpha 3 | | |
| G027 | subunit (GNA3) | M27543; J03198 | P08754 |
| | guanine nucleotide-binding protein G(Y) alpha 11 | | P29992; Q14350; |
| G037 | subunit (GNA1 1) | M69013 | O15109 |
| H001 | delta 7-dehydrocholesterol reductase (DHCR7) | AF034544 | Q60492 |
| | sequalene monooxygenase (SQLE); squalene | | |
| H004 | epoxidase (SE); ERG1 | D78130 | Q14534 |
| H008 | hydroxymethylglutaryl-CoA lyase L07033 | P35914 | |
| | CAAX-box farnesyltransferase alpha (FTase-alpha; | | |
| H010 | ENTA) | L10413 | P49354 |
| H022 | corticosteroid 1 1-beta-dehydrogenase isozyme 2 | U14631 | Q13194; P80365 |
| H025 | apolipoprotein Al (APOA1) | X00566 | P02647 |
| H026 | apolipoprotein CII (APQC2) | X00568 | P02655 |
| H030 | apolipoprotein B100 (APOBiQO) | X04506 | P04114 |
| H039 | mitochondrial hydroxymethylglutaryl-CoA synthase | X83618 | P54868 |
| H040 | rab geranylgeranyl transferase beta subunit | Y08201 | P53611; Q92697 |
| H055 | tissue inhibitor of metalloproteinase 3 (TIMP3) | U14394 | P35625 |
| | 3-beta-hydroxy-delta-5-steroid | | |
| | dehydrogenase/steroid delta isomerase 1 | | P14060; Q14545; |
| H058 | (HSD3B1); HSD3B2 | M27137; M67466 | P26439 |
| H073 | matrix metalloproteinase 2 (MMP2) | J03210 | P08253 |
| | sterol O-acyltransferase 1 (SOAT1); acyl-CoA: | | |
| H078 | cholesterol acyltransferase (ACAT) | L21934 | P35610 |
| | sterol regulatory element-binding transcription | | |
| H081 | factor 1 | U00968 | P36956 |
| H085 | oxysterol-binding protein (0SBP) M8691 7; | J04757 | P22059 |
| H092 | apolipoprotein CIV (AP0G4) | U32576 | P55056 |
| H094 | mitochondrial glycerol-3-phosphate dehydrogenase | U12424 | P43304 |
| H099 | alcohol sulfotransferase | L20000 | Q06520 |
| H103 | angiotensinogen (AGT) | K02215 | P01019 |
| H104 | renin | L00073 | P00797 |
| H105 | angiotensin 1 converting enzyme | J04144 | P12821 |
| H108 | endothelin 1 (ET1) | Y00749 | P05305 |
| H115 | alcohol dehydrogenase 5 clii polypeptide | M30471 | P11766 |
| H116 | alcohol dehydrogenase class II pi subunit | M15943 | P08319 |
| | alpha 1 B adrenergic receptor (ADRAI B); alpha 1 B | | |
| H134 | adrenoceptor | U03865 | P35368 |
| | hydroxysteroid 17-beta dehydrogenase 3 | | |
| | (HSD17B3); estradiol 17-beta dehydrogenase 3 | | |
| H149 | (EDHI7B3) | U05659 | P37058 |
| | brain fatty acid-binding protein (B-FABP); FABP7; | | |
| H152 | brain lipid-binding protein (BLBP) | AJ002962 | O15540; O14951 |
| H156 | caveolin2 | AF035752; U32114 | P51636 |
| | caveolin 3 (CAV3); limb-girdle muscular dystrophy | | |
| H157 | type IC (LGMD1C); VIP21 | AF043101 | P56539 |
| H159 | LDL-associated phospholipase A2 | U20157 | Q15692; Q13093 |
| | vasoactive intestinal polypeptide receptor 2 | P41587; Q15870; | |
| H166 | (VIPR2); pituitary adenylated cyclase activating | L40764 | Q13053 |
| | polypeptide receptor 3 (PACAP3 receptor; | | |
| | PACAPR3); helodermin-preferring receptor | | |
| | nuclear receptor subfamily 3 group C member 1 | | |
| | (NR3C1); lymphocyte glucocorticoid receptor (GRL; | | |
| H170 | GCR) | M10901 | P04150; P04151 |
| H178 | steroid receptor coactivator 1 (SRC1) | U59302 | Q00150 |
| | estrogen-related receptor alpha (ERR-alpha; | | |
| H180 | ESRRA); estrogen receptor-like 1 (ESRL1) | X51416; Y00290 | P11474; 014514 |
| H194 | prothrombin; coagulation factor II | V00595 | P00734 |
| | | | Q16001; Q15190; |
| H196 | autoprothrombin IIA | X02750 | Q15189; P04070 |
| H201 | amyloid-like protein 2 | S60099 | Q06481 |
| H207 | signal transducer CD24 | M58664 | P25063 |
| H209 | macrosialin | S57235 | P34810 |
| H210 | selectin P ligand | U02297 | Q14242; Q12775 |
| H218 | alpha-2-antiplasmin | D00174 | P08697 |
| H222 | GAP junction alpha-4 protein | M96789 | P35212 |
| H238 | protein 4.1 | M14993 | P11171 |
| H241 | vinculin | M33308 | P18206 |
| | ubiquitously expressed nuclear receptor (U N R; | | |
| | NER); nuclear receptor subfamily 1 group H | | |
| | member 2 (NR1 H2); oxysterols receptor LXR beta | | |
| H260 | (LXRB) | U07132 | P55055 |
| H262 | retinoid X receptor gamma (RXR-gamma; RXRG) | U38480 | P48443 |
| | | | Q13440; Q13441; |
| H266 | retinoic acid receptor alpha (RAR-alpha; RARA) | X06614 | P10276 |
| | | | Q15067; Q15068; |
| | | | Q12863; Q15101; |
| H279 | peroxisomal acyl-coenzyme A oxidase | S69189 | Q16131 |
| H295 | tumor-associated antigen L6 | M90657 | P30408 |
| | vascular ATP-diphosphohydrolase (ATPDase); | | |
| H298 | lymphoid cell activation antigen; 0039 antigen | S73813 | P49961 |
| | insulin-responsive glucose transporter type 4 | | |
| | (GLUT4); solute carrier family 2 member 4 | | |
| H312 | (SLC2A4) | M20747 | P14672 |
| H321 | cardiac muscle troponin T (TNTC) | S64668 | P45379; Q99596 |
| | sodium/potassium-transporting ATPase gamma | | |
| | subunit (Na +/K+ ATPase gamma; ATP1G1; | | |
| H328 | ATP1C); sodium pump gamma subunit | U50743 | P54710 |
| | alpha-galactosidase A; melibiase; alpha-D- | | |
| H330 | galactoside galactohydrolase | X05790 | P06280 |
| H345 | type Vcollagen alpha 2 subunit | Y14690 | P05997 |
| | | | P02452; 015176; |
| H348 | collagen 1 alpha 1 subunit (COLlA1) | K01228 | Q14037 |
| H349 | dodecenoyl-coenzyme A delta isomerase | L24774 | P42126 |
| H350 | collagen XV alpha 1 subunit (COLISA1) | L25286 | P39059 |
| H352 | collagen IValpha 1 subunit (COL4A1) | M11315 | P02462 |
| H357 | collagen V alpha 1 subunit (COL5A1) | M76729 | P20908 |
| H363 | collagen VI alpha 1 subunit (COL6A1) | X15880 | P12109 |
| H367 | cytochrome P450 11B6 (CYP2B6); CYP2B3 | M29874; J02864 | P20813 |
| | | | Q02928; Q16865; |
| H374 | cytochrome P450 IVAl 1 (CYP4A1 1); CYP4A2 | L04751 | Q16866 |
| H383 | cytochrome P450 XIXA1 (CYP19A1) | M22246 | Q16731; P11511 |
| H392 | steroidogenic acute regulatory protein | U17280 | P49675 |
| H393 | Niemann-Pick C disease protein (NPC1) | AF002020 | Q15118 |
| | platelet-activating factor acetylhydrolase IB alpha | | |
| H403 | subunit | L13387 | P43034 |
| | platelet-activating factor acetylhydrolase IB gamma | | |
| H404 | subunit | D63391 | Q15102 |
| | platelet-activating factor acetylhydrolase IB beta | | |
| H405 | subunit | D63390 | Q00687; Q29459 |
| H408 | platelet glycoprotein IX | HS1144 | P14770 |
| H417 | profilin I | J03191 | P07737 |
| | annexin VI (ANX6); lipocortin VI; protein III; | | |
| | chromobindin 20; 67-kDa calelectrin; calphobindin-II | | |
| N003 | (CPB-l1) | D00510 | P08133 |
| N004 | pyruvate kinase R/L (PKLR); PK1 | D10326 | P30613; P11973 |
| | lysosome membrane protein II (LIMP II); 85-kDa | | |
| | lysosomal membrane sialoglycoprotein (LGP85); | | |
| N005 | CD36 antigen-like 2 (CD36L2) | D12676 | Q14108 |
| | mitochondrial trifunctional protein enoyl-CoA | | |
| | hydratase/3-hydroxyacyl-CoA dehydrogenase alpha | | |
| N011 | subunit | D16480 | P40939 |
| | RAB GDP dissociation inihibitor alpha (RAB GDI | | |
| N026 | alpha); GDI-1; XAP-4 | D45021 | P31150; P50394 |
| | | | P10636; P18518; |
| N037 | microtubule-associated protein tau (MAPT); MTBTI J03778 | Q14799; Q15551 | |
| N041 | annexin VII (ANX7); synexin | J04543 | P20073 |
| | phenylalanine-4-hydroxylase (PAH); phe-4- | | |
| N045 | monooxygenase | K03020 | P00439 |
| | protein-tyrosine phosphatase 2C (PTP-2C); SH- | | |
| N060 | PTP2 | L08807 | Q06124 |
| | glycerol kinase (GK); ATP:glycerol 3- | | |
| N066 | phosphotransferase; glycerokinase | L13943 | P32189 |
| | phosphatidylinositol 4-kinase alpha (P14-kinase; | | |
| N083 | PTDINS-4-kinase; P14K-alpha) | L36151 | P42356 |
| N089 | apolipoprotein E (APOE) | M12529 | P02649 |
| | beta-hexosaminidase alpha chain; N-acetyl-beta- | | |
| N092 | glucosaminidase; beta-N-acetylhexosaminidase | M13520 | P06865 |
| N093 | myelin basic protein (MBP) | M13577 | P02686 |
| N096 | beta-glucuronidase (beta-G 1) | M15182 | P08236 |
| N099 | glutamate dehydrogenase 1 (GDH; GLUD1) | M18377 | P00367 |
| | annexin IV (ANX4); ANXA4; placental anticoagulant | | |
| N100 | protein II (PAP-I1) | M19383 | P09525 |
| | annexin III (ANX3); lipocortin III; placental | | |
| | anticoagulant protein III) (PAP-III); 35-alpha | | |
| | calcimedin; inositol 1,2-cyclic phosphate 2- | | |
| N101 | phosphohydrolase | M20560; J03899 | P12429 |
| N110 | ras-related protein RAB1A; YPT1 -related protein | M28209 | P11476 |
| N121 | ADP-ribosylation factor 1 (ARF1) | M36340 | P32889; P10947 |
| N122 | acetylcholinesterase (ACHE) | M55040 | P22303 |
| | myristoylated alanine-rich O-kinase substrate | | |
| | (MARCKS); protein kinase C substrate 80-kDa | | |
| N135 | protein light chain (PKCSL) | M68956 | P29966 |
| N139 | hexokinase 1 (HK 1); brain-form hexokinase | M75126 | P19367 |
| N142 | calpactin 1 light chain | M81457 | P47736 |
| | protein phosphatase 2C alpha isoform (PP2C- | | |
| N166 | alpha) | S87759 | P35813 |
| | tyrosine-protein kinase FRK; nuclear tyrosine | | |
| N168 | protein kinase RAK | U00803 | P42685; Q13128 |
| | protein-tyrosine phosphatase eta (R-PTP-eta); | | |
| | density-enhanced phosphatase 1 (DEP1); CD148 | | |
| N179 | antigen; PTPRJ | U10886 | Q12913 |
| N187 | lumican (LUM); keratan sulfate proteoglycan; LDC | U18728; U21128 | P51884 |
| N213 | protein phosphatase 2A Balphal regulatory subunit | U37352 | Q13362 |
| | | | O00754; Q93094; |
| N239 | LYSOSOMAL ALPHA-MANNOSIDASE | U60266 | Q16680; O15330 |
| N249 | syntaxin 7 (STX7) | U77942 | Q15400 |
| N255 | c factor receptor beta (GDNFR-beta) | U93703 | Q15316 |
| | annexin V; lipocortin V; endonexin II; caiphobindin I | | |
| | (CBP-1) ; placental anticoagulant protein 1 (PAP-1); | | |
| | PP4; thromboplastin inhibitor; vascular | | |
| N263 | anticoagulant-alpha (VAC-alpha; anchorin CII | X12454 | P08758 |
| | fur; furin; paired basic amino acid residue cleaving | | |
| N273 | enzyme (PACE); dibasic processing enzyme | X17094 | P09958; Q14336 |
| | diacylglycerol kinase alpha (DGK-alpha; DAG | P23743; O75481; | |
| N288 | kinase alpha); 80-kDa diglyceride kinase | X62535 | Q75482; Q75483 |
| | mannosyl-oligosaccharide alpha-1,2-mannosidase; | | |
| N304 | Man9-alpha-mannosidase | X74837 | P33908 |
| | microsomal triglyceride transfer protein (MTP) 97- | | |
| N305 | kDa subunit | X75500 | P55157 |
| N315 | calcium-binding protein ERC-55 | X78669 | Q14257 |
| | serine/threonine protein phosphatase 5 (PP5); | | |
| N331 | protein phosphatase T (PPT) | X89416 | P53041; Q16722 |
| | DNA polymerase gamma (POLG); mitochondrial | | |
| N344 | DNA polymerase catalytic subunit (MDP1) | X98093 | P54098; Q92515 |
| | Alzheimer's disease amyloid A4 protein; protease | | |
| N348 | nexin-II (PN-II); APPI | Y00264 | P05067; P09000 |
| | dolichyl-diphosphooligosaccharide-protein | | |
| N349 | glycosyltransferase 67-kDa subunit; ribophorin I | Y00281 | P04843 |
| N356 | vesicle-membrane fusion protein SNAP23A | Y09567 | Q00161 |
| N364 | laminin alpha 2 subunit (LAMA2); laminin M chain | Z26653 | P24043; Q14736 |
| | (LAMM); merosin heavy chain | | |
| | 43-kDa postsynaptic protein; acetyicholine receptor- | | |
| N366 | associated 43-kDa protein; RAPSYN | Z33905 | Q13702 |
| N378 | glial fibrillary acidic protein (GFAP) | J04569 | P14136 |
| N381 | dynamin 1 | L07807 | Q05193 |
| | beta-adaptin 1; plasma membrane adaptor | | |
| | HA2JAP2 adaptin beta subunit; clathrin assembly | | |
| | protein complex 2 beta large subunit; AP10SA; | | |
| N385 | ATDB1; BAM22 | L13939 | Q10567; P78436 |
| | dystroglycan; dystrophin-associated glycoprotein 1 | | |
| N389 | (DAG1) | L19711 | Q14118 |
| N393 | dynamin 2 | L36983 | P50570 |
| N396 | creatine kinase M subunit | M14780 | P06732 |
| N401 | casein kinase II alpha subunit (CK I1); CSNK2A2 | M55268 | P19784 |
| | protein phosphatase PP2A 55-kDa regulatory | | |
| | subunit neuronal isoform; protein phosphatase | | |
| N409 | PP2A B subunit beta; beta-PR55 | M64930 | Q00005 |
| | | | P22307; Q16622; |
| N412 | sterol carrier protein 2 (SCP2) | M75883 | Q15432 |
| N416 | somatostatin receptor 2 (SSTR2; SS2R); SRIF1 | M81830 | P30874 |
| | glutamate decarboxylase 67-kDa isoform; 67-kDa | | |
| N418 | glutamic acid decarboxylase (GAD-67); GAD1 | M81883 | Q99259 |
| | endothelial nitric oxide synthase (EC-NOS; ENOS); | P29474; Q14251; | |
| N424 | type III NOS | M93718 | Q14434 |
| | MAP kinase-activated protein kinase 2 (MAPKAP | | |
| N430 | kinase 2; MAPKAPK-2) | U12779 | P49137 |
| | tyrosine 3-monooxygenase/tryptophan 5- | | |
| | monooxygenase activation protein epsilon | | |
| | polypeptide (YWHAE); 14-3-3 protein epsilon; | | |
| | protein kinase C inhibitor protein 1 (KCIP1); | | P42655; P29360; |
| N433 | mitochondrial import stimulation factor L subunit | U28936 | Q63631 |
| | serine/threonine protein phosphatase PP2A-alpha | | |
| N442 | catalytic subunit | X12646 | P05323; P13197 |
| | cAMP-dependent protein kinase type II alpha | | |
| N446 | regulatory subunit (PRKAR2A; PKR2) | XI4968 | P13861; Q16823 |
| | phosphatidylinositol-4-phosphate 5-kinase type III | | |
| | (PIP5K3); PTDINS(4)P-5-kinase C isoform; | | |
| | diphosphoinositide kinase + phosphatidylinositol-4- | | |
| | phosphate 5-kinase type II alpha (PIP5K2A); | | |
| N460 | PTDINS(4)P-5-kinase B isoform | S78798 + U14957 | P53807 + P48426 |
| N466 | ras-related protein RAB5B | X54871 | P35239; P35277 |
| | 14-3-3 protein beta/alpha; protein kinase C inhibitor | | |
| N469 | protein-1 (KCIP-1); protein 1054 | X57346 | P31946 |
| N474 | caltractin 1 (CALT); centrin 2 (GETN2; CEN2) | X72964 | P41208 |
| N478 | ras-related protein RAB11B; YPT3 | X79780 | Q15907 |
| N479 | casein kinase 1 alpha 1 (CSNKIA1) | X80693 | P48729 |
| | serine/threonine protein phosphatase beta 1 | | |
| N480 | catalytic subunit (PP1CB; PPPIB; PP1B) | X80910 | P37140 |
| N488 | protein kinase C zeta type (NPKC-zeta) | Z15108 | Q05513 |
| | basic helix-loop-helix domain-containing protein | | |
| | class B2 (BHLHB2); differentiated embryo | | |
| P003 | chondrocyte-expressed gene 1 (DEC1) | AB004066 | Q14503 |
| P007 | zinc finger protein UBID4 | AF001433 | Q92785 |
| | segment polarity protein dishevelled homolog 2 | | |
| P008 | (DVL2) | AF006012 | Q14641 |
| P011 | mothers against dpp homolog 7 (SMAD7; MADH7) | AF015261 | Q14740 |
| | POD1 mesoderm-specifc basic helix-loop-helix | | |
| P014 | protein | AF035718 | Q43545 |
| | cysteine- & glycine-rich protein 2 (CSRP2; CRP2); | | |
| P022 | smooth muscle cell LIM protein (SMLIM) | D42123 | P52943 |
| | early growth response protein 2 (EGR2); KROX20 | | |
| P026 | homolog | J04076 | P11161 |
| | sex-determining region Y box-containing gene 2 | | |
| P028 | (SRY box-containing gene 2; SOX2) | L07335 | P48431; Q14537 |
| | | | Q14762; Q15719; |
| P036 | presenilin 1 (PSNLI; PSEN1; PS1) | L42110 | Q15720; P49768 |
| P038 | presenilin 2 (PSEN2; PSNL2; PS2) | L43964 | P49810 |
| | inhibin beta B subunit (INHBB); activin beta B | | |
| P040 | subunit | M31682 | P09529 |
| | TCF1 homeobox protein; hepatocyte nuclear factor | | |
| | 1 alpha (HNFIA); liver-specific transcription factor | | |
| P043 | LBB1 | M57732 | P20823 |
| P046 | MAX protein | M64240 | P25912; P52163 |
| | SEF2-1B protein; helix-loop-helix DNA-binding | P15884; Q15439; | |
| P048 | protein | M74719 | Q15440 |
| | sex-determining region Y box-containing gene 5 | | |
| P056 | (SRY box-containing gene 5; SOX5) | 583308 | P35711 |
| P060 | homeobox protein MOX-1 | U10492 | P50221 |
| P063 | CHUK helix-loop-helix protein | U22512 | Q13132; Q92467 |
| P085 | caudal-type homeobox protein 2 (CDX2); CDX3 | U51096 | Q00503; Q99626 |
| | | | Q15910; Q92857; |
| P086 | enhancer of zeste homolog 2 (EZH2) | U52965 | Q15755 |
| | hepatocyte nuclear factor 3 forkhead homolog 4 | | |
| P102 | (HFH4) | U69537 | Q92949; Q00630 |
| | | | O00167; Q99503; |
| P107 | eyes absent homolog 2 (EYA2) | U71207 | Q99812 |
| | transforming growth factor beta 4 (TGF-beta 4); | | |
| P116 | endometrial bleeding-associated factor | U81523 | Q00292 |
| P117 | homeobox protein B13 (H0XB13) | U81599 | Q99810; Q92826 |
| P118 | eyes absent homolog 3 (EYA3) | U81602 | Q99504; Q99813 |
| | neurogenic locus notch protein homolog 3 | | |
| P124 | (NOTCH3) | U97669 | none |
| | secreted phosphoprotein 1 (SSP1); osteopontin | | |
| | (OPN); bone sialoprotein 1 (BSPI; BNSP); early T- | | P10451; Q15681; |
| P126 | lymphocyte activation protein 1 (ETA1) | X13694 | Q15682; Q15683 |
| P137 | homeobox protein 8 (HOX8); MSX2 | X69295 | P35548 |
| | homeobox protein MOX-2; growth arrest-specific | | |
| P144 | homeobox protein | X82629 | P50222 |
| | | | Q15672; Q92487; |
| P151 | twist-related protein | X99268 | Q99804 |
| P162 | caudal-type homeobox protein 1 (CDX1) | U51095 | P47902 |
| | paired mesoderm homeobox protein 1 (PHOXI; | | |
| P169 | PMX1); aristaless homolog | M95929 | P54821 |
| P176 | CTG3a; numb-like protein | U80758 | none |
| | sex-determining region Y box-containing gene 9 | | |
| | (SRY box-containing gene 9; SOX9); campomelic | | |
| | dysplasia protein 1 (CMPDI; CMD1); autosomal | | |
| P186 | sex-reversal protein 1 (SRA1) | Z46629 | P48436 |
| | neurofilament triplet L protein (NFL); 68-kDa | | |
| P193 | neurofilament protein | X05608 | P07196; Q16154 |
| | neurofilament triplet H protein (NFH); 200-kDa | | |
| P194 | neurofilament protein | X15306 | P12036 |
| | syndecan2 (SYND2); fibroglycan; heparine; sulfate | | |
| P198 | proteoglycan core protein 0 (HSPG) | J04621 | P34741 |
| | nck, ash & phospholipase C gamma-binding protein | | |
| S1 | (NAP4) | AB005216 | O14512 |
| S10 | DNA polymerase beta (POLB) | D29013 | P06746 |
| S100 | DNA topoisomerase II beta (TOP2B) | X68060 | Q02880 |
| | N-oxide-forming dimethylaniline monooxygenase 4; | | |
| S101 | hepaticflavin-containing monooxygenase 4 (FMO4) | Z11737 | P31512 |
| | xeroderma pigmentosum group F complementing | | |
| | protein (XPF); excision repair cross-complementing | | |
| | rodent repair deficiency complementation group 4 | | |
| S102 | (ERCC4); ERC01 1 | L77890 | Q92889; Q00140 |
| | 70-kDa heat shock protein 5 (HSPA5); 78-kDa | | |
| | glucose-regulated protein (GRP78); | | |
| S103 | immunoglobulin heavy chain binding protein (BIP) | M19645 | P11021 |
| S104 | V(D)J recombination activating protein 2 (RAG2) | M94633 | P55895 |
| | DNA mismatch repair protein PMS1 (PMS1 protein | | |
| S105 | homolog 1) | U13695 | P54277 |
| | DNA mismatch repair protein PMS2 (PMS1 protein | | |
| S106 | homolog 2) | U13696 | P54278 |
| | replication protein A 30-kDa subunit; replication | | |
| S107 | factor A protein 4 (RPA4; RFA) | U24186 | Q13156 |
| S108 | mutY homolog (hMYH) | U63329 | Q15830 |
| S109 | beta crystallin A4 (CRYBA4) | U59057 | P53673 |
| | T-complex protein 1 epsilon subunit (TCP1- | | |
| S11 | epsilon); CCT-epsilon (CCTE; CCT5) | D43950 | P48643 |
| S111 | beta crystallin BI (CRYBB1) | U35340 | P53674 |
| S112 | beta crystallin B2 (CRYBB2); BP | L10035 | P43320 |
| S113 | beta crystallin 83 (CRYBB3; CRYB3) | U71216 | P26998; Q92965 |
| S114 | B-lymphocyte germinal center kinase (GC kinase) | U07349 | Q12851 |
| | cytochrome P450 11A6 (CYP2A6); CYP2A3; | M33318; X13930; | P11509; P10890; |
| S115 | coumarin 7-hydroxylase | X13897 | P00190; Q16803 |
| | 3-mephenytoin 4 hydroxylase; cytochrome P450 | M21940; M15331; | P11712; P11713; |
| | IIC9 (CYP2C9); CYP2C10; CYP2C17; CYP2C18; | M21939; M61858; | P33259; P33260; |
| S116 | CYP2C19 | M61854 | P33261 |
| | interferon-inducible RNA-dependent protein kinase | | |
| S117 | (P68 kinase) | M35663; U50648 | P19525 |
| | mitochondrial heat shock 10-kDa protein (HSP10); | | |
| S118 | HSPE1; 10-kDa chaperonin (CPN10) | U07550 | Q04984; Q95421 |
| | heat shock 17-kDa protein (HSP17); HSPL27; | | |
| S119 | HSPB3 | U15590 | Q12988 |
| S12 | probable protein disulfide isomerase P5 | D49489 | Q15084 |
| | 90-kDa heat-shock protein beta (HSP90); 84-kDa | | |
| S120 | heat-shock protein beta (HSP84); HSPCB | M16660 | P08238 |
| | heat shock 70-kDa protein 6; heat shock 70-kDa | | |
| S121 | protein B | X51757; M11236 | P48741 |
| | heat shock cognate 71-kDa protein; heat shock 70- | | |
| S122 | kDa protein 8 (HSPA8; HSC70); HSP73 | Y00371 | P11142 |
| S123 | heat shock-related 70-kDa protein 2 | L26336 | P54652 |
| S124 | cytochrome P450 IIE1 (CYP2E1) | J02625 | P05181 |
| S125 | cytochrome P450 IIF1 (CYP2F1) | J02906 | P24903 |
| | microsomal UDP-glucuronosyltransferase 1-6 | | |
| S126 | (UDPGT; UGTI.6; UGT1F; GNT1) | J04093 | P19224 |
| S127 | glutathione S-transferase mu 3 (GSTM3); GST5 | J05459 | P21266 |
| | cytochrome P450 IAl (CYPIA1); P450-P1; P450 | | |
| S128 | form 6; P450-C | K03191 | P04798 |
| | peroxisome proliferatitve-activated receptor alpha | | Q07869; Q92689; |
| S129 | (PPAR-alpha; PPARA) | L02932 | Q16241; Q92486 |
| S13 | protein disulfide isomerase-related protein (PDIR) | D49490 | Q14554 |
| | soluble epoxide hydrolase (SEH); cytosolic epoxide | | |
| | hydrolase (CEH; EPHX2; EPH2); epoxide | | |
| S130 | hydratase | L05779 | P34913 |
| | liver carboxylesterase; acyl coenzyme A:cholesterol | | P23141; Q16737; |
| | acyltransferase (ACAT); monocytelmacrophage | Q14062; Q13657; | |
| S131 | serine esterase (hMSE); CES2 | L07765 | Q00015 |
| | serum paraoxonase/arylesterase 2 (PON2); serum | | |
| | aryldiakylphosphatase 2; aromatic esterase 2 (A- | | Q15165; O15114; |
| S132 | esterase 2) | L48513 | O15115; O75856 |
| | serum paraoxonase/arylesterase 3 (PON3); serum | | |
| | aryldiakylphosphatase 3; aromatic esterase 3 (A- | | Q15166; O75855; |
| S133 | esterase 3) | L48516 | O76060 |
| | cytochrome P450 XXIB (CYP21 B); CYP21A2; | | P08686; P04033; |
| S134 | steroid 21-hydroxylase | M12792; M23280 | Q01204 |
| | mitochondrial cytochrome P450 XIA1 (CYP11A1); | | |
| S135 | P450 cholesterol side chain cleavage (P450SCC) | M14565 | P05108 |
| | cytochrome P450 11D6 (CYP2D6); P450-DB1; | | |
| S136 | debrisoquine 4-hydroxylase | M20403 | P10635; Q16752 |
| | microsomal UDP-glucuronosyltransferase 1-1 | | |
| | (UDPGT; UGTI.1; UGTIA; GNT1); bilirubin-specific | | |
| S138 | isozyme 1 (hUG-BR1) | M57899 | P22309 |
| S139 | microsomal UDP-qlucuronosyltransferase 1-4 | M57951 | P22310 |
| | (UDPGT; UGTI.4; UGTID; ONT1); bilirubin- | | |
| | specific isozyme 2 (hUG-BR2) | | |
| S14 | YSK1; Ste20 & SPS1-related kinase D63780 000506 | | |
| | serum paraoxonase/arylesterase 1 (PON1); serum | | |
| | aryldiakylphosphatase 1; aromatic esterase 1 (A- | | |
| S140 | esterase 1) | M63012 | P27169 |
| | membrane-bound & soluble catechol-0- | | |
| S141 | methyltransferase (COMT) | M65212 | P21964 |
| S142 | monoamine oxidase A (MAOA) | M68840 | P21397; Q16426 |
| | flavin-containing amine oxidase B; monoamine | | |
| S143 | oxidase (MAOB) | M69177 | P27338 |
| | eukaryotic peptide chain release factor subunit 1 | | |
| S145 | (ERF1); TB3-1; Cli protein | M75715 | P46055 |
| | microsomal UDP-glucuronosyltransferase 1-3 | | |
| S146 | (UDPGT; UGTI.3; UGTIC; GNT1) | M84127 | P35503 |
| | structure-specific recognition protein 1 (SSRP1); | | |
| | recombination signal sequence recognition protein; | | |
| S147 | T160 | M86737 | Q08945 |
| | microsomal UDP-glucuronosyltransferase 1-2 | | |
| S148 | (UDPGT; UGTI.2; UGT1B; ONT1); HLUGP4 | S55985 | P36509 |
| | | | P51580; O43213; |
| | | | O14806; O15515; |
| S149 | thiopurine 5-methyltransferase (TPMT) | S62904 | O15423 |
| | meiotic recombination protein DMC1/LIM15 | | |
| S15 | homolog | D63882 | Q14565; Q99498 |
| | short/branched chain-specific acyl-CoA | | |
| | dehydrogenase (SBCAD; ACADSB); 2-methyl | | |
| | branched chain acyl-CoA dehydrogenase (2- | | |
| S150 | MEBCAD) | U12778 | P45954 |
| | polymorphic arylamine N-acetyltransferase (PNAT) | | |
| S151 | + monomorphic (MNAT) | X14672; X17059 | P18440 |
| | glutathione peroxidase-gastrointestinal (GSHPX- | | |
| | 01); glutathione peroxidase-related protein 2 | | |
| S152 | (GPRP) | X53463 | P18283 |
| | cytochrome P450 XIB1 (CYPIIB1); steroid 11- | | |
| S153 | beta-hydroxylase (SI 1 BH) | X55764 | P15538 |
| S154 | cytochrome P450 IVAl 1 (CYP4A1 1) | X71480 | Q06766 |
| S156 | bleomycin hydrolase (BLM hydrolase) | X92106 | Q13867 |
| S157 | diaphorase 1 (DIA1); cytochrome b5 reductase | Y09501 | P00387 |
| | coproporphyrinogen III oxidase (CPO); | | |
| S158 | coproporphyrinogenase; coprogen oxidase (COX) | Z28409 | P36551 |
| | 11 0-kDa heat-shock protein (HSP110); 105-kDa | | |
| S16 | heat-shock protein (HSP105); KIAAO201 | D86956 | Q92598; Q95739 |
| | gamma crystallin C (CRYGO; CRYG3; gamma | | |
| | crystallin 2 + gamma crystallin B (CRYOB; | U66582 + M11971; | |
| S160 | CRYG2); gamma crystallin 1-2 | M11970 | P07315 + P07316 |
| S17 | heat shock transcription factor 4 (HHSF4) | D87673 | Q99472 |
| S18 | cytochrome P450 IVBI (CYP4B1); P450-HP | J02871 | P13584 |
| S19 | copper-zinc-containing superoxide dismutase 3 | J02947 | P08294 |
| | (Cu-Zn SOD3); extracellular SOD (ECSOD) | | |
| S2 | DNAJ protein homolog 2 (DNAJ2; hDJ2; HSJ2) | D13388 | P31689 |
| | DNA mismatch repair protein MSH3; divergent | | |
| | upstream protein (DUP); mismatch repair protein 1 | | |
| S20 | (MRP1) | J04810 | P20585; Q92867 |
| S21 | protein disulfide isomerase-related protein ERP72 | J05016 | P13667 |
| | replication protein A 32-kDa subunit (RPA32); | | |
| S22 | replication factor A protein 2 (RFA; RPA2) | J05249 | P15927 |
| | mu-crystallin homolog (CRYM); NADP-regulated | | |
| S23 | thyroid hormone-binding protein (THBP) | L02950 | Q14894 |
| | ATP-binding cassette subfamily C member 1 | | |
| | (ABCC1); multidrug resistance-associated protein 1 | | |
| S24 | (MRP1) | L05628 | P33527; Q14819 |
| | replication protein A 14-kDa subunit (RP-A) (RF-A); | | |
| S25 | replication factor A protein 3 | L07493 | P35244 |
| | calnexin (CANX); major histocompatibility complex | L10284; L18887; | |
| S26 | class 1 antigen-binding protein p88; 1P90 | M94859; M98452 | P27824 |
| | cyclophilin 40 (CYP4Q; CYPD); 40-kDa peptidyl- | | |
| | prolyl cis-trans isomerase (PPlase); rotamase; | | |
| S27 | cyclophilin-related protein | Li 1667 | Q08752 |
| | heat shock 70-kDa protein 4 (HSPA4); HSP70RY; | | |
| S28 | heat shock 70-related protein APG-2 | L12723 | P34932; 095756 |
| | quinone oxidoreductase; NADPH:quinone | | |
| S29 | reductase; zeta-crystallin (CRYZ) | L13278; S58039 | Q08257 |
| | T-complex protein 1 theta subunit (TCPI-theta); | | |
| S3 | CCT-theta (CCTQ; CCT8); KIAA00O2 | D13627 | P50990 |
| | mitochondrial stress-70 protein; 75-kDa glucose- | | |
| | regulated protein (GRP75); peptide-binding protein | | P38646; P30036; |
| S30 | 74 (PBP74); mortalin (MOT); HSPA9B | L15189 | P31932 |
| | p23; 23-kDa progesterone receptor-associated | | |
| S31 | protein | L24804; L24805 | Q15185 |
| | FLAP endonuclease 1 (FEN 1); maturation factor 1 | | |
| S33 | (MF1) | L37374 | P39748 |
| | DNA nucleotidylexotransferase; terminal addition | | |
| | enzyme; terminal deoxynucleotidyltransferase | | |
| S34 | (DNTT; TDT) | M11722; K01919 | P04053 |
| | poly(ADP-ribose) polymerase (PARP; ADPRT; | | |
| | ADPRP; PPOL); NAD(+) ADP-ribosyltransferase; | | |
| S35 | poly(ADP-ribose) synthetase | M18112; J03473 | P09874 |
| S36 | V(D)J recombination activating protein 1 (RAG1) | M29474 | P15918 |
| | | | M34539; M80199; |
| | FK506-binding protein 12 (FKBP12); peptidyl-prolyl | M92423; X55741; | |
| S37 | cis-trans isomerase (PPlase); rotamase | X52220 | P20071 |
| | ATP-dependent DNA ligase 1 (LIG 1); | | |
| S38 | polydeoxyribonucleotide synthase | M36067 | P18858 |
| | FK506-binding protein 13 (FKBP13); FKBP2; | | |
| S39 | peptidyl-prolyl cis-trans isomerase (PPlase) | M65128 | P26885 |
| | xeroderma pigmentosum group A complementing | | |
| S4 | protein (XPA) | D14533 | P23025 |
| S40 | heat shock transcription factor 2 (HSTF2; HSF2) | M65217 | Q03933 |
| | DNA-3-methyladenine glycosylase (ADPG); 3- | | |
| | alkyladenine DNA glycosylase; N-methylpurine- | | |
| S41 | DNA glycosirase (MPG) | M74905 | P29372 |
| S42 | DNA polymerase delta catalytic subunit | M80397 | P28340 |
| | caireticulin (CRP55); cairegulin; HACBP; ERP60; | | |
| S43 | 52-kDa ribonucleoprotein autoantigen R01SS-A | M84739 | P27797 |
| | stress-induced phosphoprotein 1 (STIP1); | | |
| | HSP70/HSP90-organizing protein (HOP); IEF-SSP- | | |
| S44 | 3521 | M86752 | P31948 |
| | alpha crystallin B subunit (alpha(B)-crystallin; | | |
| S45 | CRYAB; CRYA2); Rosenthal fiber component | S45630 | P02511 |
| | heat shock protein beta 2 (HSPB2); DMPK-binding | | |
| S46 | protein; MKBP | S67070 | Q16082 |
| S47 | alpha crystallin A chain (CRYAA; CRYA1) | U05569 | P02489 |
| S48 | nicotinamide N-methyltransferase (NNMT) | U08021 | P40261 |
| | microsomal UDP-glucuronosyltransferase 2B 15 | U08854; X63359; | |
| | (UDPGT); UDPGTH-3; UGT2BI5 + microsomal | U06641; J05428; | |
| S49 | 2B10 (UDPGT); UGT2B10 + 2microsomal B8 | Y00317 | P54855 |
| | probable protein disulfide isomerase ER-60 | | |
| | (ERP60); 58-kDa microsomal protein; | D16234; Z49835; | |
| S5 | phospholipase C alpha (PLC-alpha; PLCA) | D83485; U42068 | P30101 |
| | phenol-sulfating phenol sulfotransferase 1 (PPST1); | | |
| | thermostable phenol sulfotransferase (TS-PST); | | |
| | HASTI/HAST2; ST1A3; STP1+ PPST2; ST1A2; | | |
| | STP2 + monoamine-sulfating phenol | U09031 + U28170 + | P50225 + P50226 + |
| S50 | sulfotransferase | L19956 | P50224 |
| | NADP+ dihydropyrimidine dehydrogenase (DPD); | | |
| | dihydrouracil dehydrogenase; dihydrothymine | | Q12882; Q16694; |
| S51 | dehydrogenase (OPYD) | U09178 | Q16761 |
| | transcriptional regulator atrX; X-linked helicase II | | P46100; P51068; |
| S52 | (XH2); X-linked nuclear protein (XNP); RAD54L | U09820 | Q15886 |
| | tumor necrosis factor type 1 receptor-associated | | |
| S53 | protein (TRAP1) | U12595 | Q12931; O75235 |
| | 265 proteasome regulatory subunit S2 (PSMD2); | | |
| | tumor necrosis factor type 1 receptor-associated | | Q13200; Q12932; |
| S54 | protein (TRAP2); 55.11 protein | U12596 | Q15321 |
| | damage-specific DNA-binding protein p127 subunit | | |
| S55 | (DDBA p127); DOBi | U18299 | Q16531 |
| | damage-specific DNA binding protein p48 subunit | | |
| | (DDBB P48); implicated in xeroderma pigmentosum | | |
| S56 | group E (DDB2) | U18300 | Q92466 |
| | cockayne syndrome group A; WO-repeat protein | | |
| S57 | (CSA protein) | U28413 | Q13216 |
| | HSC70-interacting protein; progesterone receptor- | | |
| S58 | associated P48 protein | U28918 | P50502 |
| | T-complex protein 1 delta subunit (TCP1-delta); | | |
| | CCT-delta (CCTD; CCT4); stimulator of tar RNA | | |
| S59 | binding (SRB) | U38846 | P50991; O14870 |
| | 7,8-dihydro-8-oxoguanine triphosphatase (8-oxo- | | |
| S6 | dGTPase); mutT homolog 1 (MTH1) | D16581 | P36639 |
| | X-ray repair-complementing defective repair in | | |
| S60 | Chinese hamster cells 4 (XRCC4) | U40622 | Q13426 |
| S61 | DNA topoisomerase III alpha (TOP3A) | U43431 | Q13472; Q13473 |
| S62 | G/T mismatch-specific thymine DNA glycosylase | U51166 | Q13569 |
| | (TDG) | | |
| S63 | 150-kDa oxygen-regulated protein (ORPI50) | U65785 | none |
| | X-ray repair-complementing defective repair in | | |
| S64 | Chinese hamster cells 9 (XRCC9) | U70310 | O15287 |
| S65 | 48-kDa FKBP-associated protein (FAP48) | U73704 | Q92990 |
| S66 | endonuclease III homolog 1; HNTH1; OCTS3 | U79718 | Q99794 |
| | T-complex protein 1 eta subunit (TCP1-eta); COT- | | |
| S67 | eta (COTH; CCT7); HIV-1 NEF interacting protein | U83843 | Q99832; O14871 |
| S68 | catalase (CAT) | X04076 | P04040 |
| | porphobilinogen deaminase (PBGD); | | |
| | hydroxymethylbilane synthase (HMBS); pre- | | P08397; P08396; |
| S69 | uroporphyrinogen synthase | X04808 | Q16012 |
| | xeroderma pigmentosum group C complementing | | |
| S7 | protein (XPC) | D21089 | Q01831 |
| S70 | heme oxygenase 1 (H01; HMOX1) | X06985 | P09601 |
| | | | P04179; P78434; |
| S71 | Mn+ superoxide dismutase 2 (SOD2) | X07834; X59445 | Q16792 |
| | tumor rejection antigen 1 (TRA1); GP96 homolog; | | |
| | 94-kDa glucose-regulated protein (GRP94); | | |
| S72 | endoplasmin | X15187; M33716 | P14625 |
| S73 | uracil-DNAglycosylase (UNG1) | X15653 | P13051 |
| S74 | NCK melanoma cytoplasmic src homolog (HSNCK) | X17576 | P16333 |
| | 5,6-dihydroxyindole-2-carboxylic acid oxidase | | |
| | (DHICA oxidase); tyrosinase-related protein 1 | | |
| S75 | (TRP-1); catalase B; glycoprotein-75 (GP75) | X51420 | P17643 |
| S76 | uracil-DNA glycosylase 2 (UNG2) | X52486 | P22674 |
| | T-complex protein 1 alpha subunit (TCP1-alpha); | | |
| S77 | COT-alpha (COTA; COT1) | X52882 | P17987; Q15556 |
| S78 | 40S ribosomal protein S3 (RPS3) X55715 | P23396 | |
| | DNA-(apurinic or apyrimidinic site) lyase; AP | | |
| | endonuclease 1 (APE1); apurinic/apyrimidinic | | |
| | endonuclease (APEX); APEX nuclease (APEN); | | |
| S79 | REF1 | X59764; X66133 | P27695; Q99775 |
| S8 | heme oxygenase 2 (H02; HMOX2) | D21243; S34389 | P30519; O60605 |
| | collagen-binding protein 1 (CBP1); CBP2; colligin 1; | | |
| S80 | colligin 2; heat shock 47-kDa protein (HSP47) | X61598; D83174 | P29043; P50454 |
| | T-complex protein 1 gamma subunit (TCP1- | | |
| S82 | gamma); OCT-gamma (CCTG; CCT3); TR10S | X74801; U17104 | P49368 |
| | DNA damage repair & recombination protein 54 | | |
| S83 | homolog (RAD54) | X97795 | Q92698 |
| | transcription factor IIH (TFIIH); 52-kDa basic | | |
| S84 | transcription factor 2 subunit (BTF2p52) | Y07595 | Q92759 |
| | X-ray repair-complementing defective repair in | | |
| S85 | Chinese hamster cells 2 (XRCC2) | Y08837 | O43543 |
| | | | O15527; O00390; |
| | 8-oxyguanine DNA glycosylase 1 (OGG1); mutM | P78554; O00670; | |
| S86 | homolog (MMH) | Y11838 | O00705; O14876 |
| S87 | cytochrome P450 1A2 (CYPIA2) | Z00036 | P05177; Q16754 |
| | basic transcription factor 2 34-kDa subunit | | |
| S88 | (BTF2p34) | Z30093 | Q13889 |
| | N-oxide forming dimethylaniline monooxygenase 5; | | |
| | hepatic flavin-containing monooxygenase 5 | | |
| S89 | (FMO5); dimethylaniline oxidase 5 | L37080 | P49326 |
| S9 | ubiquitin-like protein NEDD8 | D23662 | Q15843 |
| | multidrug resistance protein 3 (MDR3); P- | | |
| S91 | glycoprotein 3 (PGY3) | M23234 | P21439 |
| | N-oxide forming dimethylaniline monooxygenase 1; | | |
| | hepatic flavin-containing monooxygenase 1 | | |
| S92 | (FM01); dimethylaniline oxidase 1 | M64082 | Q01740 |
| | ubiquitin-conjugating enzyme E2B (UBE2B); | | |
| | ubiquitin-protein ligase; ubiquitin carrier protein; | | |
| S93 | HR6B | M74525 | P23567 |
| | p59 protein; HSP-binding immunophilin (HB1); | | |
| | possible peptidyl-prolyl cis-trans isomerase | | |
| | (PPlase); rotamase; 52-kDa FK506-binding protein | | |
| S95 | (FKBP52); FKBP59; HSP56; FKBP4 | M88279 | Q02790 |
| | heat shock protein 40 homolog (HSP40 homolog); | | |
| S96 | DNAJW | U40992 | Q9UDY4 |
| | 51-kDa FK506-binding protein (FKBP51); peptidyl- | | |
| | prolyl cis-trans isomerase (PPlase); rotamase; 54- | | |
| | kDa progesterone receptor-associated | | |
| | immunophilin; FKBP54; FF1 antigen; HSP90- | | |
| S97 | binding immunophilin | U42031 | Q13451 |
| S98 | hematopoietic progenitor kinase (HPK1) | U66464 | Q92918 |
| S99 | SPS1/Ste20 homolog KHS1 | U77129 | none |
| | a disintegrin & metalloproteinase domain 12 | | |
| SK013 | (ADAM12); meltrin-S | AF023477 | O60470 |
| SK029 | plasma glutathione peroxidase (GPXP; GPX3) | D00632; X58295 | P22352 |
| | | | M16650; X55362; |
| SK035 | ornithine decarboxylase (ODC1) | M20372 | P11926 |
| SK052 | matrix gla-protein (MGP) | X07362 | P08493 |
| | melanoma-derived growth regulatory protein; | | |
| SK054 | melanoma inhibitory activity (MIA) | X84707 | Q16674 |
| | cystatin B (CSTB); liver thiol proteinase inhibitor | | |
| SK062 | (OPIB); stefin B | L03558 | P04080 |
| | chaperonin-containing T-complex polypeptide 1 | | |
| | beta subunit (COT-beta; CCTB; CCT2; TCP1- | | |
| ST005 | beta); 9908.1 | AF026293 | P78371 |
| | T-complex protein 1 zeta-like subunit (OCT-zeta- | | |
| | like; TCP1-zeta-like); TSA303; testis-specific | | |
| ST015 | TCP20 | D78333 | Q92526 |
| | thioredoxin (TROX; TXN); ATL-derived factor | | |
| | (ADF); surface-associated sulphydrylprotein | | |
| ST018 | (SASP) | J04026 | P10599 |
| ST027 | mitochondrial uncoupling protein 3 (UCP3) AF011449 | P55916; O60475 | |
| | gamma-glutamyl hydrolase (GGH; GH); | | |
| | folylpolygammaglutamyl hydrolase; gamma-glu-X | | |
| ST038 | carboxypeptidase; conjugase | U55206 | Q92820 |
| ST040 | farnesol receptor HRR-1 | U68233 | Q92943 |
| ST042 | mitochondrial uncoupling protein 2 (UCP2); UCPH | U82819 | P55851 |
| | phosphoglyceride kinase 1 (PGKI; PGKA); primer | | |
| ST046 | recognition protein 2 (PRP2) | V00572 | P00558 |
| ST051 | tyrosine protein kinase receptor UFO | X66029 | none |
| | peptidylprolyl cis-trans isomerase A (PPlase; PPIA); | | |
| | rotamase; cyclophilin A (CYPA); cyclosporin A- | | |
| ST057 | binding protein Y00052 P05092 | | |
| | muscle-specific carbonic anhydrase III (CA3); | | |
| ST065 carbonate dehydratase III | M29458 | P07451 | |
| | soluble glutamic oxaloacetic transaminase 1 | | |
| | (GOT1): cytoplasmic aspartate aminotransferase 1; | | |
| ST066 | transaminase A | M37400 | P17174 |
| | acyl-CoA dehydrogenase long chain-specific | | |
| ST069 | subunit (LCAD; ACADL) | M74096 | P28330 |
| ST075 | ribosomal protein S29 (RPS29) | U14973 | P30054 |
| | p53-induced gene 8 (PIG8); etoposide-induced | | |
| ST076 | mRNA; E124 | AF010313 | O14681 |
| | eukaryotic translation initiation factor 4 gamma 3 | | |
| ST077 | (EIF4G3) | AF012072 | O43432 |
| ST078 | glutathione S-transferase alpha 4 (GSTA4) | AF025887 | O15217 |
| | mitogen-activated protein kinase kinase kinase 1 | | |
| | (MAPKKKI; MAP3K1); MAPK/ERK kinase kinase 1 | | |
| ST079 | (MEK kinase 1; MEKK1) | AF042838 | Q13233 |
| ST080 | somatostatin (SST) | J00306 | P01166 |
| | solute carrier family 3 member 2 (SLC3A2); antigen | | |
| | identified by monoclonal antibodies 4F2, TRA1.10, | | |
| ST081 | TROP4 &T43; NACAE; MDU1; CD98 antigen | J02769 | P08195; Q13543 |
| | collagen VII alpha 1 subunit (COL7A1); dystrophic | | |
| | dominant & recessive epidermolysis bullosa protein | | Q02388; Q14054; |
| ST082 | (EBDCT) | L02870 | Q16507 |
| ST083 | core-binding factor beta subunit (CBFB); PEBP2B | L20298 | Q13951; Q13124 |
| | guanine nucleotide binding protein-like 1 (GNL1); | | |
| ST084 | HSR1 | L25665 | P36915 |
| | chaperonin containing TCPI subunit 6A (CCT6A); | | |
| | CCT-zeta 1 (CCTZ); TCP20; histidine transport | | |
| ST085 | regulator 3 (HTR3) | L27706 | P40227 |
| | coagulation factor VII (F7); serum prothrombin | | |
| ST086 | conversion accelerator | M13232 | P08709 |
| | gamma-glutamyltransferase 1 (GGT 1); glutamyl | | |
| ST087 | transpeptidase | M24903 | P19440 |
| | | | P01236; Q15199; |
| ST088 | prolactin (PRL) | V00566 | Q92996 |
| | major histocompatibility complex class II DN alpha | | |
| ST089 | (HLA-DNA); HLA-DOA; HLA-DZA | M31525 | P06340 |
| ST091 | even-skipped homeobox protein 2 homolog (EVX2) | M59982 | Q03828 |
| | carcinoembryonic antigen-related cell adhesion | | |
| ST092 | molecule 1 (CEACAM1); biliary glycoprotein (BGP) | S71326 | Q16170 |
| | GATA-binding protein 1 (GATA1); globin | | |
| | transcription factor 1 (GF1); erythroid transcription | | |
| ST093 | factor 1 (ERYF1) | X17254 | P15976 |
| | granzyme B (GZMB); granzyme 2; cytotoxic T- | | |
| | lymphocyte-associated serine esterase 1 (CTLA1); | | |
| | fragmentin 2; cathepsin G-like protein 1 (CGL1); | | |
| ST094 | CSPB | M17016; M38193 | P10144 |
| ST095 | cardiac-specific homeobox protein (CSX); NKX2-5 | U34962 | P52952 |
| | myeloid/lymphoid leukemia translocated to 10 | | |
| | homolog (MLLT 10); mixed-lineage leukemia | | |
| | translocated to 10 homolog; ALLi fused gene from | | |
| ST096 | chromosome 10(AF10) | U13948 | P55197 |
| | cyclin-dependent kinase inhibitor 1B (CDKN1B); | | |
| ST099 | CDKN4; p27KlP1 | U10906 | P46527; Q16307 |
| ST100 | ankyrin 3 node of Ranvier (ANK3); ankyrin G | U13616 | Q12955 |
| | | | L78833; U14680; |
| ST101 | early-onset breast cancer protein 1 (BRCA1) | U37574 | P38398 |
| | cyclin-dependent kinase inhibitor 2C (CDKN2C); | | |
| ST102 | p18INK4C | AF041248 | P42773 |
| | fas-associated via death domain protein (FADD); | | |
| | mediator of receptor-induced toxicity protein I | | |
| ST103 | (MORT1) | U24231 | Q13158; Q14866 |
| | growth factor-independent protein 1 (GFII); | | |
| ST104 | ZNF163 | U67369 | Q99684 |
| | karyopherin beta 2 (KPNB2); importin beta 2; | | Q92973; Q92957; |
| ST105 | transportin (TRN); M9-interacting protein (MIP) | U70322 | Q92975 |
| | telomerase-associated protein 1 (TP1); telomerase | | |
| ST106 | protein component 1 (TEP1) | U86136 | Q99973 |
| | pentraxin 3 (PTX3); pentaxin-related gene rapidly | | |
| ST108 | induced by IL1-beta | X63613 | P26022 |
| | neuronal DNAJ-like heat shock protein 1 (HSJ1); | | |
| ST109 | 40-kDa heat shock protein 3 (HSPF3) | X63368 | P25686 |
| | dual specificity phosphatase 1 (DUSP1); CL100; | | |
| | PTPN10; HVHI; MAP kinase phosphatase 1 | | |
| ST110 | (MKP1) | U01669; X68277 | P28562 |
| | | | Q15853; Q15852; |
| | fos-interacting upstream stimulatory factor 2 | Q00671; Q00709; | |
| ST111 | (USF2); FIP | Y07661 | Q05750; Q07952 |
| | insulin promoter factor 1 (IPF1); islet/duodenum | | |
| | homeobox protein 1 (IDX1); somatostatin | | |
| | transcription factor 1 (STF1); maturity-onset | | |
| ST112 | diabetes of the young protein IV (MODY4); | X99894 | P52945; Q60594 |
| | pancreas/duodenum homeobox protein 1 (PDX1) | | |
| ST113 | inhibitor of DNA-binding protein 4 (ID4) | U28368 | P47928; Q13005 |
| 51114 | thrombospondin 4 (THBS4) | Z19585 | P35443 |
| ST115 | cyclin F (CCNF) | Z36714 | P41002 |
| | 63-kDa FK506-binding protein 9 (FKBP9); FKBP63; | | |
| ST117 | FKBP60 | AF089745 | Q95302 |
| ST118 | FK506-binding protein | AF092137 | Q9Y6B0 |
| | peroxisomal biogenesis factor 11 alpha (PEX11- | | |
| ST119 | alpha; PEXiIA) | AF093668 | Q75192 |
| | cysteine desulfurase; nitrogen-fixing bacteria S | | |
| ST120 | homolog (N1FS) | AF097025 | Q9Y697 |
| | cathelicidin antimicrobial peptide (CAMP); FALL39; | | |
| ST121 | CAP18 | Z38026 | P49913 |
| ST122 | caspase 14 (CASP14); MICE | AF097874 | Q95823 |
| ST123 | timeless homolog (TIM) | AF098162 | Q9UNS1 |
| ST124 | period homolog 2 (PER2) | AB002345 | Q15055 |
| ST125 | BTB & CNC homology protein 1 (BACH1) | AB002803 | Q14867; Q43285 |
| | sex-determining region Y box-containing gene 20 | | |
| ST126 | (SRY box-containing gene 20; SOX20) | AB006867 Q60248 | |
| | heat shock transcription factor 2-binding protein | | |
| ST127 | (HSF2BP) | AB007131 | Q75031 |
| | transforming growth factor beta 1-induced transcript | | |
| ST128 | 1 (TGFBII1); HICS; ARA55; TSC5 | AB007836 | Q43294 |
| ST130 | KIAA0516 | AB011088 | Q60271 |
| | DEAD/H (Asp-Glu-AIa-Aspf His) box polypeptide 16 | | |
| ST131 | (DDX16) | AB011149 | Q60231; Q60322 |
| | | | O75771; O60355; |
| | | | O43537; O76085; |
| ST132 | RAD51-like protein 3 (RAD5IL3); TRAD | AF034956 | Q75196; Q75847 |
| ST133 | aquaporin 8 (AQP8) | AB013456 | Q94778 |
| ST134 | KIAA0700 | AB014600 | O75182 |
| | KIAA0709; macrophage mannose endocytic | | |
| ST135 | receptor | AB014609 | Q9UBG0 |
| ST136 | MRJ member of the DNAJ protein family | AB014888 | Q75190; Q95806 |
| | adaptor-related protein complex 1 sigma 2 subunit | | |
| | (AP1S2); clathrin adaptor complex 1 sigma lB | | |
| ST137 | subunit | AB015320 | none |
| ST139 | KIAAI027; talin (TLN) | AB028950 | Q9UPX3 |
| | phosphatidylethanolamine N-methyltransferase | | |
| ST140 | (PEMT) | AB029821 | Q9Y6V9 |
| ST141 | choline kinase-like protein (CHKL) | AB029886 | Q9Y259; Q13388 |
| | degenerative spermatocyte homolog (DEGS); | | |
| ST142 | membrane fatty acid (lipid) desaturase (MLD) | AF002668 | Q15121 |
| | estrogen receptor-binding fragment-associated | | |
| | gene 9 (EBAG9; EB9); receptor-binding cancer | | |
| ST143 | antigen expressed on 5150 cells 1 (RCAS1) | AF006265 Q00559 | |
| ST144 | outer dense fiber of sperm tails protein 2 (ODF2) | AF012549 | Q14721 |
| ST146 | palmitoyl-protein thioesterase 2 (PPT2) | AF020544 | Q14799 |
| | eukaryotic translation initiation factor 3 subunit 4 | | |
| ST147 | (EIF3S4); EIF3-delta | AF020833 | Q75821; Q14801 |
| ST148 | DJ-1 RNA-binding protein regulatory subunit | AF021819 | Q14805 |
| ST149 | period homolog 1 (PER1); RIGU1 | AF022991 | Q15534 |
| ST150 | deleted in liver cancer protein 1 (DLC1) | AF026219 | Q14868 |
| | branched chain alpha-ketoacid dehydrogenase | | |
| ST151 | kinase (BOKOK) | AF026548 | Q14874 |
| ST152 | microsomal glutathione 5-transferase 3 (MGST3) | AF026977 | Q14880 |
| ST153 | glypican 4 (GPC4) | AF030186 | O75487; Q9UPD9 |
| | testis-enhanced gene transcript (TEGT); BAX | | |
| ST154 | inhibitor 1 (BI1) | AF033095 | P55061; Q14938 |
| ST155 | stromal cell-derived factor receptor 1 (SDFR1) | AF035287 | Q9Y499 |
| ST156 | RING zinc finger protein 13 (RNF13; RZF) | AF037204 | Q43567 |
| ST157 | voltage-dependent anion channel 3 (VDAC3) | AF038962 | Q9Y277; Q9UISO |
| ST158 | cell death-inducing OFFA-like effector A (CIDEA) | AF041378 | O60543 |
| ST1 59 unc51 -like kinase 1 homolog (ULK1) | AF045458 | Q75385 | |
| | ubiquitin-activating enzyme EIC (UBEIC); UBA3 | | |
| ST160 | homolog | AF046024 | Q76088 |
| | amyloid beta A4 protein-binding family A member 2 | | |
| | (APBA2); X11-like protein (XIL); Munc18-1- | | |
| ST161 | interacting protein 2 (MINT2) | AF047348 | Q99767; Q60571 |
| ST162 | 32-kDa thioredoxin-like protein (TXNL; TXL) | AF051896 | Q43396 |
| ST163 | Sec6l-gamma (SEC61G) | AF054184 | none |
| | microsomal NAD+-dependent retinol | | |
| ST164 | dehydrogenase 4 (RODH4) | AF057034 | Q75452 |
| | serine protease 21 (PRSS2 1); testisin 1 (TEST1); | | |
| ST165 | serine protease from eosinophils 1 (ESP1) | AF058300 | Q9Y6M0 |
| | mitochondrial ribosomal protein SI 2 (RPMS12; | | |
| ST166 | RPSM12) | AF058761 | Q15235 |
| | ADP-forming succinate-CoA ligase beta subunit | | |
| ST167 | (SUCLA2) | AF058953 | Q95194 |
| ST168 | E2F transcription factor 6 (E2F6) | AF059292 | Q75461; Q60544 |
| ST169 | UDP-glucose dehydrogenase (UDPGDH; UGDH) | AF061016 | Q60701; Q60589 |
| | catenin delta 1 (CTNND 1); cadherin-associated src | | |
| ST170 | substrate (GAS) | AF062333 | Q60935 |
| | heart & neural crest derivatives-expressed protein 1 | | |
| ST171 | (HAND1); THING1 | AF061756 | Q96004 |
| | clone 24651 (IMAGE Consortium human infant | | |
| ST172 | brain library IN1B) | AF070648 | none |
| ST173 | SURF-4 | AF078866 | Q9UNZ1 |
| ST174 | growth factor-independent protein 1B (GFI1B) | AF081946 | Q95270 |
| | D-type cyclin-interacting protein 1 (DIP1); GCIP; | | |
| ST175 | MAID | AF082569 | Q95273 |
| | thyroid receptor-1nteracting protein 15 (TRIP 15); | | |
| ST176 | ALIEN; SGN2 | AF084260 | none |
| | Housekeeping Genes | | |
| | liver glyceraldehyde 3-phosphate dehydrogenase | | |
| 16 | (GAPDH; G3PDH) | X01677 | P04406 |
| | | | P02570; Q11211; |
| | | | P99021; Q64316; |
| 49 | cytoplasmic beta-actin (ACTB) | X00351 | P70514 |
| 901 | tubuiin alpha 1 (TUBA1) | K00558 | P04687 |
| 902 | major histocompatibility complex class 1 C (HLAC) | M11886 | P10321 |
| | 23-kDa highly basic protein; 60S ribosomal protein | | |
| 903 | L13A(RPL13A) | X56932 | P40429 |
| 904 | 40S ribosomal protein 39 (RPS9) | U14971 | P46781 |
| 905 | ubiquitin C (UBC) | M26880 | none |
| | phospholipase A2; tyrosine 3- | | |
| | monooxygenase/tryptophan 5-monooxygenase | | |
| | activation protein zeta polypeptide (YWHAZ); 14-3- | | |
| | 3 protein zeta/delta; protein kinase C inhibitor | | |
| | protein 1 (K01P1); factor activating exoenzyme S | | |
| 906 | (FAS) | M86400 | P29312; P29213 |
| | hypoxanthine-guanine phosphoribosyltransferase 1 | | |
| 907 | (HPRT1) | V00530 | P00492 |

### EXAMPLE 3

### Alternative Human Stress Array

An alternative array to that described in Examples 1 and 2 displays probes obtained from the list of genes appearing in Table 3, where these particular probes are displayed instead of or in addition to those described in Tables 1 and/or 2.

TABLE 3

| Clon ID | Gene Name | Gen Bank Acc # | Sprot Acc # |
|---|---|---|---|
| | inducible nitric oxide synthase (INOS); type | | |
| 10 | II NOS; hepatocyte NOS (HEP-NOS) | L09210 | P35228; Q16692 |
| | 605 ribosomal protein L6 (RPL6); TAX- | | |
| | responsive enhancer element binding | | |
| | protein 107 (TAXREB107); neoplasm- | | |
| 103 | related protein C140 | X69391 | Q02878 |
| | activating transcription factor 4 (ATF4); tax- | | |
| | responsive enhancer element B67 | | |
| | (TAXREB67); cAMP-response element- | | |
| 145 | binding protein 2 (CREB2) | D90209 | P18848 |
| | platelet-derived growth factor B subunit | | |
| | precursor (PDGFB; PDGF2); bacaplermin; | X02811; X02744; | |
| 15 | c-sis | M12783; M16288 | P01127; P78431 |
| | tumor necrosis factor receptor 1 (TNFR1); | | |
| | tumor necrosis factor binding protein 1 | | |
| 155 | (TBP1); CD120A antigen | M33294 | P19438 |
| | hepatocyte growth factor activator (HGF | | |
| 167 | activator) | D14012 | Q04756; Q14726 |
| | interleukin-1 alpha precursor (IL-1 alpha; | | |
| 18 | IL1A); hematopoietin-1 | X02851 | P01583 |
| | glial growth factor 2 precursor | | |
| | (GGFHPP2); neuregulin; heregulin-beta3 + | L12260; L12261 + | Q07110; Q07111 + |
| 183 | neu differentiation factor + heregulin-alpha | U02326 + M94165 | Q12780 + Q02297 |
| | T-cell-specific rantes protein precursor; sis | | |
| | delta; small inducible cytokine A5 (SCYA5); | | |
| 195 | rantes pro-inflammatory cytokine | M21121 | P13501 |
| | interferon gamma precursor (IFN-gamma; | | |
| 2 | IFNG); immune interferon | X01992; M29383 | P01579 |
| 206 | oncostatin M (OSM) | M27288 | P13725 |
| | interleukin-4 precursor (IL-4); B-cell | | |
| | stimulatory factor 1 (BSF-1); lymphocyte | | |
| 21 | stimulatory factor 1 | M13982 | P05112 |
| | interleukin-6 precursor (IL-6); B-cell | | |
| | stimulatory factor 2 (BSF2); interferon beta- | | |
| 22 | 2 (IFNB2); hybridoma growth factor | X04602; M14584 | P05231 |
| | hepatocyte growth factor (HGF); scatter | | |
| 226 | factor (SF); hepatopoeitin A | M60718 | P14210 |
| | tumor necrosis factor alpha precursor | | |
| 23 | (TNF-alpha; TNFA); cachectin | X01394 | P01375 |
| | interleukin-12 beta subunit precursor (IL- | | |
| | 12B); cytotoxic lymphocyte maturation | | |
| | factor 40-kDa subunit (CLMF p40); NK cell | | |
| 237 | stimulatory factor subunit 2 (NKSF2) | M65290 | P29460 |
| | interleukin-12 alpha subunit precursor (IL- | | |
| | 12A); cytotoxic lymphocyte maturation | | |
| | factor 35-kDa subunit (CLMF p35); NK cell | | |
| 238 | stimulatory factor subunit 1 (NKSF1) | M65291 | P29459 |
| | fasL receptor; apoptosis-mediating surface | | |
| 239 | antigen fas; APO-1 antigen; CD95 antigen | M67454 | P25445 |
| | lymphotoxin-alpha precursor (LT-alpha); | | |
| | tumor necrosis factor-beta (TNF-beta; | | |
| 24 | TNFB) | D12614 | P01374 |
| | interferon gamma-induced protein | | |
| 273 | precursor (gamma-IP10) | X02530 | P02778 |
| | interleukin-5 precursor (IL-5); T-cell | | |
| | replacing factor (TRF); eosinophil | | |
| | differentiation factor; B-cell differentiation | | |
| 28 | factor I | X04688; J03478 | P05113 |
| | macrophage inflammatory protein 2 alpha | | |
| | (MIP2-alpha); growth-regulated protein beta | | |
| 288 | (GRO-beta) | X53799 | P19875 |
| | OX40 ligand (OX40L); GP34; tax- | | |
| | transcriptionally activated glycoprotein 1 | | |
| 298 | (TXGP1) | X79929 | P23510 |
| | granulocyte-macrophage colony stimulating | | |
| 31 | factor (GM-CSF); CSF2 | M11220 | P04141 |
| | integrin alpha 9 (ITGA9); integrin alpha- | | |
| 318 | RLC | D25303; L24158 | Q13797; Q14638 |
| | intercellular adhesion molecule 1 precursor | | |
| | (ICAM1); major group rhinovirus receptor; | | |
| 329 | CD54 antigen | J03132 | P05362 |
| | transforming growth factor-beta 3 (TGF- | | |
| 330 | beta3) | J03241 | P10600 |
| | natural killer cell enhancing factor (NKEFB) + | | |
| | thiol-specific antioxidant protein (TSA); | | |
| | thioredoxin peroxidase 1 (TDPX1); | | |
| | thioredoxin-dependent peroxide reductase | L19185 + Z22548; | P32119; P35701 |
| 362 | 1 | X82321 | P31945; Q92763 |
| | protein serine/threonine kinase STK1; cell | | |
| | division protein kinase 7 (CDK7); CDK- | | |
| | activating kinase (CAK); 39-kDa protein | | |
| 366 | kinase | L20320 | P50613 |
| 373 | apoptosis regulator bax | L22474 | Q07814 |
| | placental plasminogen activator inhibitor 2 | | |
| | (PAI-2; PLANH2); monocyte ARG-serpin; | | |
| 396 | urokinase inhibitor | M18082; J02685 | P05120 |
| 408 | ras-related protein RAB5A | M28215 | P20339 |
| | epidermal growth factor precursor (EGF); | | |
| 41 | beta-urogastrone | X04571 | P01133 |
| | E-selectin precursor (SELE); endothelial | | |
| | leukocyte adhesion molecule 1 (ELAM1); | | |
| | leukocyte-endothelial cell adhesion | | |
| 416 | molecule 2 (LECAM2); CD62E antigen | M30640 | P16581 |
| | NADH-ubiquinone dehydrogenase 1 beta | | |
| | subcomplex 7 18-kDa subunit (NDUFB7); | | |
| | complex I-B18 (CI-B18); cell adhesion | | |
| 425 | protein SQM1 | M33374 | P17568 |
| | interleukin-10 precursor (IL-10); cytokine | | |
| 43 | synthesis inhibitory factor (CSIF) | M57627 | P22301 |
| | octamer-binding transcription factor 2 (oct- | | |
| | 2; OTF2); lymphoid-restricted | | |
| | immunoglobulin octamer binding protein | | |
| 435 | NF-A2; POU2F2 | M36542 | P09086 |
| | transcriptional enhancer factor (TEF1); | | |
| | protein GT-IIC; transcription factor 13 | | |
| 462 | (TCF13) | M63896 | P28347 |
| | ubiquitin-conjugating enzyme E2 17-kDa | | |
| | (UBE2A); ubiquitin-protein ligase; ubiquitin | | |
| 468 | carrier protein; HR6A | M74524 | P49459 |
| 483 | 40S ribosomal protein S19 (RPS19) | M81757 | P39019 |
| | peroxisome assembly factor 1 (PAF1); | | |
| | peroxisomal membrane protein 3 (PXMP3; | | |
| | PMP3); 35-kDa peroxisomal membrane | | |
| 490 | protein (PMP35); peroxin 2 (PEX2) | M86852 | P28328 |
| 532 | amphiphysin (AMPH) | U07616 | P49418 |
| 59 | erythropoietin receptor (EPOR) | M60459 | P19235 |
| 6 | interleukin-2 precursor (IL-2); T-cell growth | A14844 | P01585 |
| | factor (TCGF) | | |
| | 5-hydroxytryptamine 1D receptor (5-HT-1D; | | |
| 624 | HTR1D); serotonin receptor | M89955 | P28221 |
| | cyclin-dependent protein kinase 2 (CDK2); | | |
| 632 | p33 protein kinase | M68520 | P24941 |
| | colorectal cancer suppressor protein | | |
| 665 | precursor (DCC) | X76132 | P43146 |
| | fibronectin receptor alpha subunit (FNRA); | | |
| | integrin alpha 5 (ITGA5); VLA5; CD49E | | |
| 687 | antigen | X06256 | P08648 |
| | integrin alpha L (ITGAL); leukocyte | | |
| | adhesion glycoprotein alpha subunit | | |
| | precursor; leukocyte function-associated | | |
| | molecule 1 alpha chain (LFA1); CD11A | | |
| 693 | antigen | Y00796 | P20701 |
| | fas antigen ligand (FASL); apoptosis | | |
| 694 | antigen ligand (APTL; APT1LG1); TNFSF6 | D38122; U08137 | P48023 |
| 712 | L-myc proto-oncogene (MYCL1) | M19720 | P12524 |
| 726 | transcription factor relB; I-rel | M83221 | Q01201 |
| 759 | fos-related antigen (FRA1); fosL1 | X16707 | P15407 |
| 763 | vimentin (VIM) | X56134; M14144 | P08670 |
| | tumor necrosis factor receptor 1-associated | | |
| | death domain protein (TNFR1-associated | | |
| 781 | death domain protein; TRADD) | L41690 | Q15628 |
| 782 | apoptosis regulator bcl-2 | M14745 | P10415 |
| | TNF-related apoptosis inducing ligand | | |
| 797 | (TRAIL); APO-2 ligand (APO2L) | U57059 | P50591 |
| 85 | DNA-binding protein inhibitor ld-2 | M97796 | Q02363 |
| | CDC25B; CDC25HU2; M-phase inducer | | |
| 878 | phosphatase 2 | M81934; S78187 | P30305 |
| | cyclin-dependent kinase 4 inhibitor 2B | | |
| | (CDKN2B); p14-INK4B; multiple tumor | | |
| 879 | suppressor 2 (MTS2) | U17075; L36844 | P42772 |
| | triiodothyronine receptor; thyroid hormone | | |
| | receptor (THRA1); v-erbA-related protein | | |
| 97 | ear-1 | M24898 | P20393 |
| AF104419 | decoy receptor 3 (DCR3) | AF104419 | O95407 |
| B121 | dihydrofolate reductase (DHFR) | V00507 | Q14130; P00374 |
| B122 | thymidylate synthase (TYMS; TS) | X02308 | P04818 |
| | octamer-binding transcription factor 1 (oct- | | |
| | 1; OTF1); octamer binding protein NF-A1; | | |
| B132 | POU2F1 | X13403 | P14859 |
| | zinc finger protein 40 (ZNF40); human | | |
| | immunodeficiency virus type I enhancer- | | |
| | binding protein 1 (HIV-EP1); major | | |
| | histocompatibility complex binding protein 1 | | |
| | (MBP-1); positive regulatory domain II | | |
| B138 | binding factor I (PRDII-BF1) | X51435 | P15822 |
| | major vault protein (MVP); lung resistance- | | |
| B164 | related protein (LRP) | X79882 | Q14764 |
| | nervous-system specific octamer-binding | | |
| | transcription factor N-oct3; N-oct5A & N- | | |
| | oct5B; brain-specific homeobox/POU | | |
| B179 | domain protein 2 (POU3F2); brn2; oct7 | Z11933 | Q14960; P20265 |
| | B-lymphocyte kinase; tyrosine-protein | | |
| B188 | kinase BLK; p55-BLK | Z33998 | P51451 |
| B25 | cytosolic thymidine kinase (TK1) | K02581 | P04183 |
| | plasma-cell membrane glycoprotein PC-1; | | |
| | alkaline phosphodiesterase I; nucleotide | | |
| B70 | pyrophosphatase (NPPase) | M57736 | P22413 |
| | matrix metalloproteinase 14 precursor | | |
| | (MMP14); MMP-X1; membrane-type matrix | | |
| C121 | metalloproteinase 1 (MT-MMP1) | D26512; X83535 | P50281; Q92678 |
| | cadherin 6 precursor (CDH6); kidney | | |
| C122 | cadherin (K-cadherin) | D31784 | P55285 |
| | cadherin 11 precursor (CDH11); osteoblast- | | |
| C141 | cadherin (OB-cadherin); OSF4 | L34056 | P55287; Q15065 |
| | cadherin 12 (CDH12); brain cadherin | | |
| | precursor (Br-cadherin); neural cadherin 2 | | |
| C142 | (N-cadherin 2) | L34057; L33477 | P55289 |
| | cadherin 13 (CDH13); truncated cadherin | | |
| | precursor (T-cadherin); heart cadherin (H- | L34058; U59289; | |
| C143 | cadherin) | U59288 | P55290 |
| | cell division protein kinase 4; cyclin- | | |
| C149 | dependent kinase 4 (CDK4); PSK-J3 | M14505 | P11802; O00576 |
| | cell division protein kinase 5 (CDK5); tau | | |
| | protein kinase II catalytic subunit (TPKII | | |
| | catalytic subunit); serine/threonine protein | | |
| C201 | kinase PSSALRE | X66364 | Q00535 |
| C236 | c-myc binding protein MM-1 | D89667 | Q99471 |
| C288 | sonic hedgehog (SHH) | L38518 | Q15465 |
| | growth inhibitory factor (GIF); | | |
| C3 | metallothionein-III (MT-III; MT3) | D13365; M93311 | P25713 |
| C359 | CD27BP (Siva) | U82938 | O15304 |
| C369 | apoptosis inhibitor survivin | U75285 | O15392 |
| | interleukin-1 receptor antagonist protein | | |
| C38 | precursor (IL-1RA; IRAP) | M63099 | P18510 |
| C41 | G1/S-specific cyclin C | M74091 | P24863 |
| | serine/threonine-protein kinase PLK1 | | |
| C53 | (STPK13) | U01038 | P53350 |
| | met proto-oncogene; hepatocyte growth | | |
| | factor receptor precursor (HGF-SF | | |
| C7 | receptor) | J02958 | P08581 |
| C79 | CDC37 homolog | U63131 | Q16543 |
| | tissue inhibitor of metalloproteinase 1 | | |
| | precursor (TIMP1); erythroid potentiating | | |
| C83 | activity (EPA); fibroblast collagenase | X03124 | P01033; Q14252 |
| | inhibitor | | |
| | retinoic acid receptor alpha; retinoid X | | |
| C92 | receptor alpha (RXRA) | X52773 | P19793 |
| | matrix metalloproteinase 11 (MMP11); | | |
| C95 | stromelysin 3 | X57766 | P24347 |
| CA033 | macrophage inhibitory cytokine 1 (MIC1) | AF019770 | Q99988 |
| CA061 | RecQ protein-like (DNA helicase Q1-like) | D37984 | P46063 |
| CA079 | calmegin | D86322 | O14967 |
| | ribosomal protein S6 kinase II alpha 1 | | |
| | (S6KII-alpha 1); ribosomal S6 kinase 1 | | |
| CA102 | (RSK1) | L07597 | Q15418 |
| | major histocompatibility complex enhancer- | | |
| CA169 | binding protein MAD3 | M69043 | P25963 |
| | hypoxia-inducible factor 1 alpha (HIF1 | | |
| | alpha); ARNT-interacting protein; member | | |
| CA219 | of PAS protein 1 (MOP1) | U22431 | Q16665 |
| CA260 | DNA-binding protein UEV-1; UBE2V | U49278 | Q13532 |
| CA270 | cell cycle protein P38-2G4 homolog; HG4-1 | U59435 | O43846 |
| CA280 | JV18-1. HMAD-2 OR MADR2 OR SMAD2 | U68018 | Q15796 |
| CA282 | box-dependent myc-interacting protein 1 | U68485 | Q92944 |
| CA283 | fuse-binding protein 2 (FBP2) | U69126 | Q92945 |
| | btg protein precursor; NGF-inducible anti- | | |
| CA287 | proliferative protein PC3 | U72649 | P78543 |
| | sentrin; ubiquitin-like protein SMT3C; | | |
| | ubiquitin-homology domain protein PIC1; | | |
| | UBL1; SUMO-1; GAP modifying protein 1; | | |
| CA295 | GMP1 | U83117 | Q93068 |
| CA359 | L-lactate dehydrogenase H subunit (LDHB) | Y00711 | P07195 |
| CA388 | methyl CpG-binding protein 2 (MECP2) | L37298 | P51608; O15233 |
| | GLCLC, GLCL (Glutamate-cysteine ligase | | |
| | catalytic subunit, gamma-glutamylcysteine | | |
| CA396 | synthetase) | M90656 | P48506 |
| | methallothionein IH (MT1H); | | P80294 + P02795 + |
| | metallothinein0 (MT0) + MT1I; MT2 + MT1L + | X64177 + X97260 + | P80297 + |
| CA410 | MT1R | X76717 + X97261 | Q93083 |
| CA421 | checkpoint kinase 1 (CHK1) | AF016582 | O14757 |
| CA458 | erythropoietin | M11319 | P01588 |
| | AP4 basic helix-loop-helix DNA-binding | | |
| CA466 | protein | S73885 | Q01664 |
| CA467 | fatty acid synthase | S80437 | Q13479 |
| | H-ras proto-oncogene; transforming G | | |
| CA514 | protein | V00574 | P01112 |
| CA517 | ornithine decarboxylase | X16277 | P11926 |
| CA527 | high mobility group protein HMG2 | X62534 | P26583 |
| | aurora- & IPL1-like midbody-associated | | |
| CA552 | protein kinase 1 (AIM1); ARK2 | AF008552 | O60446 |
| E026 | RCL growth-related c-myc-responsive gene | AF040105 | O43598 |
| | putative renal organic anion transporter 1 | | |
| E044 | (hROAT1) | AF057039 | O95187 |
| E050 | cyclin K | AF060515 | O75909 |
| | | AF039067; | |
| E061 | IEX-1L anti-death protein; PRG-1; DIF-2 | AF071596 | O75353 |
| E084 | pancreatitis-associated protein 1 precursor | D13510 | Q06141 |
| E091 | proteasome activator HPA28 subunit beta | D45248 | Q15129 |
| E097 | thiosulfate sulfurtransferase; rhodanese | D87292 | Q16762 |
| E099 | cyclin G-associated kinase (GAK) | D88435 | O14976 |
| E105 | alpha-1-antichymotrypsin precursor (ACT) | K01500 | P01011 |
| | neutral amino acid transporter A (SATT); | | |
| | alanine/serine/cysteine/threonine | | |
| E112 | transporter (ASCT1) | L14595 | P43007 |
| E114 | monocarboxylate transporter 1 (MCT1) | L31801 | P53985 |
| E122 | neuromedin B precursor | M21551 | P08949 |
| | cystic fibrosis transmembrane conductance | | |
| | regulator (CFTR); cAMP-dependent | | |
| E124 | chloride channel | M28668 | P13569 |
| E127 | deoxyribonuclease I (DNase I) | M55983 | P24855 |
| | canalicular multispecific organic anion | | |
| | transporter; multidrug resistance- | | |
| | associated protein 2 (MRP2); canalicular | | |
| E166 | multidrug resistance protein | U63970 | Q92887 |
| | alpha-1-antitrypsin precursor; alpha-1 | | |
| E179 | protease inhibitor; alpha-1-antiproteinase | X02920 | P01009 |
| | hepatocyte nuclear factor 4 (HNF4); | | |
| E195 | transcription factor 14 | X76930 | P41235 |
| E197 | metal-regulatory transcription factor | X78710 | Q14872 |
| | chloride conductance regulatory protein | | |
| | ICLN; nucleotide-sensitive chloride channel | | |
| | 1A; chloride ion current inducer protein | | |
| E200 | (CLCI); reticulocyte PICLN | X91788 | P54105 |
| | eosinophil granule major basic protein | | |
| | precursor (MBP); pregnancy-associated | | |
| | major basic protein; bone marrow | | |
| E204 | proteoglycan 2 | Y00809 | P13727 |
| | dual-specificity protein phosphatase 9; | | |
| | mitogen-activated protein kinase | | |
| | phosphatase 4 (MAP kinase phosphatase 4 | | |
| E207 | (MKP4) | Y08302 | Q99956 |
| | CCAAT/enhancer binding protein alpha | | |
| E226 | (C/EBP alpha) | U34070 | P49715 |
| | NAD(P)H dehydrogenase; quinone | | |
| | reductase; DT-diaphorase; azoreductase; | | |
| | phylloquinone reductase; menadione | | |
| E240 | reductase | J03934 | P15559 |
| H028 | apolipoprotein A-II precursor (APOAII) | X00955 | P02652 |
| H029 | apolipoprotein C-III precursor (APOCIII) | X01388 | P02656 |
| H031 | hepatic triglyceride lipase (HTGL) | X07228 | P78529; P11150 |
| H044 | NADPH-cytochrome p450 reductase | S90469 | Q16455; P16435 |
| H046 | adrenodoxin | M34788 | P10109 |
| | tissue inhibitor of metalloproteinase 2 | | |
| H054 | precursor (TIMP2) | J05593 | P16035 |
| H063 | matrix metalloproteinase 15 (MMP15) | Z48482 | P51511 |
| H065 | matrix metalloproteinase 14 (MMP14) | D26512 | P50281 |
| H072 | matrix metalloproteinase 1 (MMP1) | X54925 | P03956 |
| | ileal sodium-dependent bile acid transporter | | |
| | (ISBT); ileal sodium/taurocholate | | |
| | cotransporting polypeptide (NTCP2); | | |
| H091 | SLC10A2 | U10417 | Q12908; Q13839 |
| | alcohol dehydrogenase alpha subunit + | | |
| | alcohol dehydrogenase 2 + alcohol | M12271 + D00137 + | P07327 + P00325 + |
| H093 | dehydrogenase 3 | X04299 | P00326 |
| H102 | bile-salt-activated lipase | M85201; M37044 | P19835 |
| | alcohol dehydrogenase 6 + aldehyde | | |
| H117 | dehydrogenase 1 (ALDH1) | K03000 | P28332 + P00352 |
| H130 | vascular cell adhesion protein 1 | X53051 | P19320 |
| | macrophage colony stimulating factor 1 | | |
| H138 | (CSF1; MCSF) | M37435 | P09603 |
| H147 | estradiol 17 beta-dehydrogenase 1 | M36263 | P14061 |
| | intestinal fatty acid-binding protein 2 | | |
| | (FABP2; IFABP) + liver fatty acid-binding | | |
| H151 | protein 1 (FABP1; LFABP) | M10050 + M10617 | P12104 + P07148 |
| H163 | corticosteroid-binding globulin | J02943 | P08185 |
| | sodium-dependent bile acid cotransporter; | | |
| | hepatic sodium/taurocholate cotransporting | | |
| H165 | polypeptide (NTCP); SLC10A1 | L21893 | Q14973 |
| | | X51473; X02415 | |
| H188 | fibrinogen G gamma polypeptide | K02569 | P02679 |
| H270 | endothelin-converting enzyme 1 | Z35307 | P42892 |
| | mitochondrial enoyl-CoA hydratase short | | |
| H273 | subunit 1 | D13900 | P30084; O00739 |
| H275 | peroxisomal bifunctonal enzyme | L07077 | Q08426 |
| H277 | orphan nuclear hormone receptor BD73 | L31785 | Q14995 |
| | very-long-chain-specific acyl-CoA | | |
| H307 | dehydrogenase precursor (VLCAD) | D43682 | P49748 |
| | familial intrahepatic cholestasis 1 protein | | |
| H339 | (FIC1) | AF038007 | O43520 |
| | peroxisomal acyl-CoA oxidase branched | | |
| H344 | subunit (BRCOX) | X95190 | Q99424 |
| | sodium-independent organic anion | | |
| | transporter; organic anion transporting | | |
| H361 | polypeptide (OATP); SLC21A3 | U21943 | P46721 |
| H365 | cytochrome P450 IB1 (CYP1B1) | U03688 | Q16678; Q93089 |
| | cytochrome P450 IIA6 (CYP2A6) + | M33318; M33316 + | P11509 + P20853 + |
| H366 | CYP2A7 + CYP2A13 + CYP2A7PT + | U22029 + U22030 + | Q16696 |
| | CYP2A7PC | U22044 | |
| | | | P05184 + P08684 + |
| | cytochrome P450 IIIA3 (CYP3A3) + | M13785 + M18907 + | P20815 + |
| H373 | CYP3A4 + CYP3A5 + CYP3A7 | J04813 + D00408 | P24462 |
| H377 | cytochrome P450 VA1 (CYPSA1) | M80647 | P24557 |
| H378 | cytochrome P450 VIIA1 (CYP7A1) | X56088 | P22680 |
| H382 | cytochrome P450 XVIIA1 (CYP17A1) | M14564 | P05093 |
| | peroxisomal 3-ketoacyl-CoA thiolase | | |
| | precursor (PTHIO); peroxisomal 3-oxoacyl- | | |
| | CoA thiolase; beta-ketothiolase; acetyl-CoA | | |
| H389 | acyltransferase (ACAA) | X14813 | P09110 |
| | low-density lipoprotein receptor (LDL | | |
| H395 | receptor; LDLR) | M28219 | P01130 |
| | prostaglandin-endoperoxide synthase 1 | | |
| | precursor; prostaglandin G/H synthase 1 | | |
| | (PGH synthase 1; PTGS1; PHS1); | | |
| H416 | cyclooxygenase 1 (COX1) | M59979 | Q15122; P23219 |
| | prostaglandin G/H synthase 2 precursor | | |
| | (PGH synthase 2; PGHS2; PTGS2); | | |
| | cyclooxygenase 2 (COX2); prostaglandin- | | |
| H418 | endoperoxide synthase 2 | M90100 | P35354; Q16876 |
| | 3-hydroxy-3-methylglutaryl-coenzyme A | | |
| N087 | reductase (HMG-CoA reductase; HMGCR) | M11058 | P04035 |
| | major prion protein precursor (PRP); | | |
| N094 | PRP27-30; PRP33-35C; ASCR | M13667 | P04156 |
| N097 | lipoprotein lipase precursor (LPL) | M15856 | P06858 |
| | lung group IB phospholipase A2 precursor | | |
| | (PLA2); phosphatidylcholine 2- | | |
| N102 | acylhydrolase | M21054 | P04054 |
| | protein phosphatase 2B regulatory subunit; | | |
| N113 | calcineurin B subunit isoform 1 | M30773 | P06705 |
| | lipopolysaccharide-binding protein | | P18428; Q92672; |
| N119 | precursor (LBP) | M35533 | O43438 |
| | protein-tyrosine phosphatase MEG2 | | |
| N144 | (PTPASE-MEG2) | M83738 | P43378 |
| | sodium- & chloride-dependent glycine | | |
| N158 | transporter 1 (GLYT-1) | S70609 | P48067 |
| | peroxisome assembly factor-2 (PAF-2); | | |
| | peroxisomal-type ATPase 1; peroxin-6; | | |
| N235 | PEX6; PXAAA1 | U56602 | Q13608; Q99476 |
| | protein-tyrosine phosphatase PTEN; | | |
| | mutated in multiple advanced cancers 1 | | |
| N253 | (MMAC1); TEP1 | U92436 | O00633; O02679 |
| | calbindin; avian-type vitamin D-dependent | | |
| N260 | calcium binding protein (CABP); D-28K | X06661 | P05937 |
| | D-amino acid oxidase (DAMOX; DAO; | | |
| N265 | DAAO) | X13227 | P14920; Q16758 |
| | dopamine beta-hydroxylase (DBH); | | |
| N266 | dopamine-beta-monooxygenase precursor | X13255 | P09172 |
| | laminin alpha 1 subunit precursor (LAMA1); | | |
| N285 | laminin A chain | X58531 | P25391 |
| | mitochondrial aldehyde dehydrogenase | | |
| N347 | precursor (class 2); ALDHI; ALDH2 | Y00109 | P05091; Q03639 |
| N395 | CREATINE KINASE B CHAIN | L47647 | P12277 |
| | protein-tyrosine phosphatase alpha | | |
| N400 | precursor (R-PTP-alpha; PTPRA; PTPA) | M34668 | P18433 |
| | activin receptor type II precursor (ACTRIIA; | | |
| P021 | ACVR2) | D31770 | P27037;; Q92474 |
| | GAP JUNCTION ALPHA-5 PROTEIN | | |
| P035 | (CONNEXIN 40) (CX40) | L34954 | P36382 |
| P039 | INVOLUCRIN | M13903 | P07476 |
| | CELLULAR RETINOIC ACID BINDING | | |
| P055 | PROTEIN | S74445 | P29762 |
| | RETINOID X RECEPTOR GAMMA (RXR- | | |
| P075 | GAMMA) | U38480 | P48443 |
| | GAP JUNCTION BETA-1 PROTEIN | | |
| | (CONNEXIN 32) (CX32) (LIVER GAP | | |
| P125 | JUNCTION PROTEIN) | X04325 | P08034 |
| SK032 | complement 3 (C3) | K02765 | P01024 |
| | polyspecific organic cation transporter N1 | | |
| ST002 | (OCTN1) | AB007448 | O14546 |
| ST003 | peroxisomal membrane protein 69 (PMP69) | AF009746 | O14678 |
| | peroxisome biogenesis disorder protein 1 | | |
| ST004 | (PEX1) | AF026086 | O43933 |
| ST006 | TNF-alpha-stimulated ABC protein (TSAP) | AF027302 | O14897 |
| | organic cation transporter-like protein 2 | | |
| ST007 | (ORCTL2) | AF037064 | O43562 |
| ST008 | organic cation transporter N2 (OCTN2) | AF057164 | O76082 |
| ST009 | sulfonylurea receptor 2A (SUR2A) | AF061323 | O60706 |
| ST111 | MRP/organic anion transporter (MOAT-B) | AF071202 | Q9Y6J2 |
| | adrenoleukodystrophy-related protein | | |
| ST012 | (ALDR) | AJ000327 | none |
| | glutamic-pyruvate transaminase 1 (GPT1); | | P24298; P78398; |
| ST014 | alanine aminotransferase 1 (AAT1) | D10355 | Q93076 |
| | vascular endothelial growth factor D | | |
| | (VEGFD); C-FOS-induced growth factor | | |
| ST016 | (FIGF) | D89630 | O43915 |
| | skeletal muscle adenine nucleotide | | |
| | translocator 1 (ANT1); heart/skeletal | | |
| | muscle ADP/ATP carrier protein isoform | | |
| ST017 | T1; ADP/ATP translocase 1 | J02966 | P12235 |
| ST019 | down-regulated in adenoma protein (DRA) | L02785 | P40879 |
| | sulfonylurea receptor (SUR); ATP-binding | | |
| | cassette subfamily C (CFTR/MRP) member | | |
| ST023 | 8 (ABCC8) | L78207 | Q09428 |
| | enolase 1 alpha (ENO1); non-neural | | |
| ST024 | enolase (NNE); phosphopyruvate | M14328 | P06733 |
| | hydratase (PPH) | | |
| | mitochondrial glutamic oxaloacetic | | |
| | transaminase 2 (GOT2); aspartate | | |
| ST025 | aminotransferase 2; transaminase A | M22632 | P00505 |
| | | | P02735; P02736; |
| ST026 | serum amyloid A1 precursor (SAA1) | M23698 | P02737 |
| | complement component 4-binding protein | | |
| | alpha (C4B-binding protein; C4BPA); | | |
| ST028 | proline-rich protein (PRP) | M31452 | P04003 |
| | mitochondrial carnitine palmitoyltransferase | | |
| ST029 | II precursor (CPTase; CPT2) | M58581 | P23786 |
| | zinc finger protein 37 (ZFP37); KRAB | | |
| ST030 | domain zinc finger protein | AF022158 | Q9Y6Q3 |
| | mitochondrial brown fat uncoupling protein | | |
| ST034 | 1 (UCP1) | U28480 | P25874; Q13218 |
| | biliverdin reductase A precursor (BLVRA; | | |
| ST035 | BVR) | U34877 | P53004 |
| | CCAAT/enhancer-binding protein epsilon | | |
| ST037 | (C/EBP epsilon; CEBPE) | U48866; U48865 | Q15744; Q15745 |
| | prostaglandin transporter (PGT); solute | | |
| ST041 | carrier family 21 member 2 (SLC21A2) | U70867 | Q92959 |
| ST044 | ATP-binding cassette transporter (ABCR) | U88667 | P78363 |
| | glucose-6-phosphate dehydrogenase | | |
| ST047 | (G6PD) | X03674 | P11413; Q16765 |
| | tyrosine aminotransferase (TAT); I- | | |
| ST048 | tyrosine:2-oxoglutarateaminotransferase | X52520 | P17735 |
| ST049 | bile salt export pump (BSEP) | AF091582 | O95342 |
| ST052 | RNase L inhibitor | X76388 | Q13181; Q13864 |
| | mitochondrial phosphoenolpyruvate | | |
| | carboxykinase 2 precursor (PEPCK-M; | | |
| ST053 | PCK2); phosphoenolpyruvate carboxylase | X92720 | Q16822; O43253 |
| | anthracycline resistance-associated protein | | |
| ST054 | (ARA) | X95715 | Q13798 |
| ST056 | kidney organic cation transporter | X98333 | O15244 |
| | 3′-phosphoadenosine 5′-phosphosulfate | | |
| | synthase 1 (PAPS synthase 1; PAPSS1); | | |
| | PAPS synthetase 1; sulfurylase kinase 1 | | O43252; O43841; |
| ST058 | (SK1) | Y10387 | O75332 |
| | multidrug resistance-associated protein 3 | | O15438; O60265; |
| ST059 | (MRP3); MLP2; ABCC3 | Y17151 | O60922 |
| | senescence marker protein 30 (SMP30); | | |
| ST062 | regucalcin (RGN; RC) | D31815 | Q15493 |
| | galactoside 2-I-fucosyltransferase 2; GDP-I- | | |
| | fucose:beta-D-galactoside 2-alpha-I- | | |
| | fucosyltransferase 2; fucosyltransferase 2 | | |
| | (FUT2); secretor blood group alpha-2- | | |
| ST063 | fucosyltransferase; secretor factor 2 (SE2) | D87942 | Q10981 |
| | ubiquitin cross-reactive protein precursor | | |
| ST064 | (UCRP); alpha-inducible interferon; | M13755 | P05161 |
| | interferon-induced 17-kDa protein; G1P2; | | |
| | ISG15 | | |
| | galactosyltransferase-associated protein | | |
| | kinase p58 (GTA); cell division cycle 2-like | | |
| ST067 | 1 (CDC2L1;CLK1) | M37712 | P21127 |
| | spermidine/spermine N1-acetyltransferase | | |
| | (SSAT); diamine acetyltransferase; | | |
| ST068 | putrescine acetyltransferase | M55580 | P21673 |
| | corticoliberin precursor; corticotropin- | | |
| | releasing factor (CRF); corticotropin | | |
| ST072 | releasing hormone (CRH) | V00571 | P06850 |
| | antigen peptide transporter 2 (APT2); | | |
| | peptide supply factor 2 (PSF2); peptide | | |
| | transporter involved in antigen processing 2 | | |
| | (TAP2); ATP-binding cassette subfamily B | | |
| | (MBR/TAP) member 3 (ABCC3); HLA class | | |
| | II histocompatibility antigen DO beta chain | X66401; L09191; | |
| ST073 | precursor | L10287 | Q03519 |

It is evident from the above results and discussion that the subject invention provides a rapid, high throughput means to simply and quickly obtain a broad-scale screening of human stress gene expression in a variety of different samples. Only simple hybridization protocols need be employed with the subject arrays, and signals can be detected using any convenient and readily available detection device. Despite their simplicity, assays conducted with the subject arrays yield a large amount of information regarding the expression of numerous different and important human stress genes associated with cellular responses to stress in a particular sample at substantially the same time. As such, the subject human stress arrays find use in a variety of different applications, including expression profiling of key human stress genes, assays designed to determine the affect of an agent on the expression of human stress genes, and the like.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A human stress array comprising a plurality of polynucleotide probe spots stably associated with the surface of a solid support, wherein each polynucleotide probe spot comprises a polynucleotide probe composition made up of unique polynucleotides corresponding to a human stress gene.

2. The array according to claim 1, wherein said unique polynucleotides of said array have an average length of from about 120 to 1000 nt.

3. The array according to claim 1, wherein each of said unique polynucleotides does not cross hybridize with a polynucleotide of any other polynucleotide probe composition on the array.

4. The array according to claim 1, wherein said polynucleotide probe composition comprises a population of single stranded identical polynucleotides.

5. The array according to claim 1, wherein said polynucleotide probe composition comprises a population of two different complementary single stranded polynucleotides.

6. The array according to claim 1, wherein the density of spots on said array does not exceed about 400/cm².

7. The array according to claim 1, wherein the number of polynucleotide probe spots on said array ranges from about 50 to 2000.

8. A human stress array comprising a plurality of 50 to 2000 polynucleotide probe spots stably associated with the surface of a solid support, wherein each polynucleotide probe spot comprises a polynucleotide probe composition made up of unique polynucleotides of from about 50 to 1000 nt in length that do not cross-hybridize with the polynucleotides of any other polynucleotide probe composition on the array and that correspond to a human stress gene.

9. The array according to claim 8, wherein said polynucleotide probe composition comprises a population of single stranded identical polynucleotides.

10. The array according to claim 8, wherein said polynucleotide probe composition comprises a population of two different complementary single stranded polynucleotides.

11. The array according to claim 8, wherein the density of spots on said array does not exceed about 400/cm².

12. The array according to claim 8, wherein at least 10 human stress genes of Tables 1, 2 and/or 3 are represented on said array.

13. A human stress array comprising from about 50 to 2000 polynucleotide probe spots stably associated with the surface of a solid support and having a density that does not exceed about 500 spots/cm², wherein said plurality of polynucleotide probe spots comprises a polynucleotide probe composition made up of unique polynucleotides of from about 50 to 1000 nt in average length and all of the unique polynucleotides of said array correspond to human stress genes, and further wherein at least 20 human stress genes listed in Tables 1, 2 and/or 3 are represented on said array.

14. The array according to claim 13, wherein each of said unique polynucleotides does not cross hybridize with the polynucleotides of any other polynucleotide probe composition on the array.

15. The array according to claim 13, wherein each of said polynucleotide spots has a diameter ranging from about 10 to 5000 μm.

16. A method of preparing a human stress array of polynucleotide probe spots, wherein each of said polynucleotide probe spots comprises a polynucleotide probe composition made up of unique polynucleotides corresponding to a human stress gene, said method comprising:
enzymatically generating said unique polynucleotides; and
stably associating said enzymatically-generated unique polynucleotides on the surface of said solid support.

17. The method according to claim 16, wherein said solid support is flexible.

18. The method according to claim 17, wherein said solid support is a nylon.

19. The method according to claim 16, wherein said solid support is rigid.

20. The method according to claim 19, wherein said solid support is glass.

21. The method according to claim 16, wherein said method further comprises the step of selecting said unique polynucleotides.

22. The method according to claim 21, wherein said unique polynucleotides are selected so that they do not cross-hybridize to any other unique polynucleotide of any other polynucleotide probe composition on the array.

23. The method according to claim 16, wherein said enzymatically generating step comprises contacting a template polynucleotide with a pair of primers, a polymerase and dNTPs under conditions sufficient for said unique polynucleotides to be produced.

24. The method according to claim 16, wherein said unique polynucleotides have an average length of from 120 to 1000 nt.

25. The array produced according to the method of claim 16.

26. A hybridization assay comprising the steps of:
contacting at least one labeled target polynucleotide sample with a human stress array according to claim 1 under hybridization conditions sufficient to produce a hybridization pattern; and
detecting said hybridization pattern.

27. The method according to claim 26, wherein said method further comprises washing said array prior to said detecting step.

28. The method according to claim 26, wherein said method further comprises preparing said labeled target polynucleotide sample.

29. The method according to claim 28, wherein said preparing comprises conjugating a detectable label to a functionalized target polynucleotide.

30. The method according to claim 26, where said method further comprises:
generating a second hybridization pattern; and
comparing said hybridization patterns.

31. The method according to claim 30, wherein said hybridization patterns are generated on the same array.

32. The method according to claim 30, wherein the second hybridization patterns are generated on different arrays.

33. A kit for use in a hybridization assay, said kit comprising:
a human stress array according to claim 1.

34. The kit according to claim 33, wherein said kit further comprises reagents for generating a labeled target polynucleotide sample.

35. The kit according to claim 33, wherein said kit further comprises a hybridization buffer.

36. The kit according to claim 33, wherein said kit further comprises a wash medium.
